(19) United States
(12) Patent Application Publication
(10) Pub. No.: US 2012/0276535 A1
(43) Pub. Date: Nov. 1, 2012

(54) METHOD FOR THE DIAGNOSIS OF LIMBAL STEM CELL DEFICIENCY

(75) Inventors: Iker Garcia Jimenez, Derio (Bizkaia) (ES); Nerea Gonzalez Fernandez, Derio (Bizkaia) (ES); Javier Soria Esponera, Derio (Bizkaia) (ES); Arantxa Acera Osa, Derio (Bizkaia) (ES); Tatiana Suárez Cortés, Derio (Bizkaia) (ES)

(73) Assignee: BIOFTALMIK, S.L., Derio (Bizkaia) (ES)

(21) Appl. No.: 13/496,766

(22) PCT Filed: Sep. 20, 2010
(86) PCT No.: PCT/EP10/63826

§ 371 Date: Jul. 11, 2012

(30) Foreign Application Priority Data
Sep. 18, 2009 (EP) 09382171.8

(51) Int. Cl. C12Q 1/68; G01N 21/64; C07H 21/04

(52) U.S. Cl. 435/6.11; 435/6.12; 536/24.33

(57) ABSTRACT

The invention relates to a method for the diagnosis of limbal stem cell deficiency (LSCD) in a subject, based on detecting or quantifying the expression of the MUC5AC gene in a cornea sample from said subject.

### FIELD OF THE INVENTION

The invention is comprised within the field of the area of diagnosis of diseases; specifically, in the development of a specific, sensitive and reliable method for the diagnosis of limbal stem cell deficiency in a subject based on the expression of the MUC5AC gene in the cornea.

### BACKGROUND OF THE INVENTION

Limbal stem cell deficiency is a clinical entity occurring due to the destruction of limbal stem cells. Said stem cells are located in an area of transition between the columnar conjunctival epithelium and the stratified squamous corneal epithelium called “limbus”. Multiple functions are developed in the limbus, such as the nutrition of the peripheral cornea, corneal healing and sensitivity responses.

These cells are believed to be responsible for the regenerative function allowing the maintenance of the corneal epithelium and for the barrier function against the migration of conjunctival cells on the cornea [Dua et al., Surv Ophthalmol (2000); 44:415-425]. The loss of said functions is known as limbal deficiency or limbal stem cell deficiency (LSCD), and can be the consequence of the direct destruction of said cell population or of its stromal microenvironment. Histopathologically, LSCD is characterized by the existence of conjunctivalization with the presence of goblet cells on the cornea, vascularization, destruction of the corneal basement membrane and chronic inflammation [Puangsricharern et al., Ophthalmology (1995); 102:1476-1485). The treatment of total LSCD requires transplanting a sufficient amount of limbal stem cells to achieve corneal reepithelialization with cells with the suitable phenotype.

The loss or dysfunction of said stem cells of the corneal epithelium in a sufficient number translates into the incapacity to maintain the dynamic equilibrium of the corneal epithelium and into the onset of the pathological condition (LSCD). When this occurs, and to prevent an epithelial defect, there is an invasion of the conjunctival epithelium in the cornea (a process known as “conjunctivalization”) which, in the absence of blood vessels, adopts a phenotype similar to the corneal phenotype although it never manages to transdifferentiate completely; this process is normally accompanied by subepithelial vascularization (with chronicity it constitutes the fibrovascular tissue known as “pannus” and the corneal transparency is altered), with persistent epithelial defects and stromal healing. In more severe cases persistent epithelial defects, calcifications, stromal ulcers and even perforations occur. Clinically, there is a loss of transparency of the cornea which, if it affects its central area, causes a decrease in visual acuity. Other symptoms with which it is associated include photophobia, lacrimation, blepharospasm, recurrent episodes of pain and chronic inflammation with reddening and edema [Annals d'Oftalmologia (2001); 9(3):149-151].

In Spain the incidence of limbus deficiency is estimated at about 5/1,000 inhabitants/year. The clinical diagnosis of LSCD must be intuited with the presence of an irregular epithelium, without shine, which stains anomalously with fluorescein (since the conjunctival epithelium is more permeable than the corneal epithelium). The presence of blood vessels which normally accompany the conjunctival epithelium makes LSCD more evident. Generally, the conjunctivalization process can theoretically be detected by the presence of goblet cells in an impression cytology of the corneal epithelium; therefore the definitive diagnosis of LSCD is currently based on a histological method for confirming the existence of an epithelium with goblet cells which are typical of the conjunctiva only and which migrate from the conjunctiva to the cornea in patients with LSCD. In fact, LSCD is currently diagnosed by means of impression cytology using PAS-hematoxylin staining and/or immunocytochemistry to detect the MUC5AC protein by means of specific antibodies in the cornea. Impression cytology is a non-invasive method for obtaining histological information, which has allowed its clinical application in the diagnosis of ocular surface pathologies. These methods are less sensitive and specific due to the material of the filters (cellulose acetate), on which the epithelium cells are collected, being stained with the PAS stain and leading to false positives, or to the preparation not being well stained and to not being able to discriminate between a goblet cell or depositions of the actual stain.

Tseng et al. [Tseng S C G et al. Am. J. Ophtalmol. (1997), 124:825-35] retrospectively studied 134 clinically suspected cases of LSCD and said suspicion could not be confirmed by means of impression cytology in 40 cases (30%) (only squamous metaplasia was detected in these cases); they were cases in which a sufficient loss of stem cells to cause a conjunctivalization of the cornea probably did not occur although there were clinical signs such as vascularization, fibrosis and epithelial defects which led to presupposing it.

Pauklin et al. [Pauklin et al. “Limbal stem cell deficiency after chemical burns: Investigations on the epithelial phenotype and inflammation status”, Ophtalmologe, (2009) Jan 24] analyze the expression of the epithelial strain markers K3, K19 and MUC5AC and of the inflammatory markers IL-1beta, ICAM-1 and VEGF by means of Western Blot and/or real-time polymerase chain reaction in the cornea and conjunctiva reaching the conclusion that the expression of K9 and MUC5AC in normal (healthy) corneal tissue was lower than the expression of said markers in normal conjunctiva.

Espana E M et al. [Espana E M et al., Br. J. Ophtalmol. 2003; 87: 1509-14] describe the use of MUC5AC as a diagnosis marker of LSCD, detecting said gene by immunofluorescence with antibodies in a cornea sample.

It is therefore necessary to develop a method for the diagnosis of LSCD which overcomes the mentioned drawbacks; it would be particularly desirable for said method to have a high sensitivity and/or specificity and to generate less false negatives than the methods usually used in the diagnosis of LSCD.

### SUMMARY OF THE INVENTION

The inventors have now found that the detection of the MUC5AC gene transcript in a sample of cornea from a subject is indicative of said subject suffering from LSCD. A number of assays conducted by the inventors have clearly shown that the detection of the MUC5AC gene transcript by means of reverse transcription (RT) and real-time polymerase chain reaction (RT-PCR) in a cornea sample from a subject, using a suitable pair of oligonucleotide primers, such as the one consisting of the oligonucleotide primer MUC5AC-RT-F2 comprising the nucleotide sequence shown in SEQ ID NO: 2 and the oligonucleotide primer MUC5AC-RT-R2 comprising the nucleotide sequence shown in SEQ ID NO: 3, allows diagnosing LSCD in a sensitive and specific manner with a higher correlation between the clinical diagnosis and the impression cytology analyzed by RT-qPCR than between the clinical diagnosis and the conventional impression cytology stained with PAS-hematoxylin (Example 1).

Therefore, in an aspect, the invention relates to an in vitro method for diagnosing limbal stem cell deficiency (LSCD) in a subject, comprising:
analyzing the expression of the MUC5AC gene in a sample of cornea from said subject, using a pair of oligonucleotide primers under conditions which allow specifically amplifying a fragment of the MUC5AC transcript, said fragment having at least 75 nucleotides long and being included within a region consisting of nucleotides 17440-18750 in the MUC5AC cDNA nucleotide sequence shown in SEQ ID NO: 1, wherein the detection of the expression of the MUC5AC gene in said sample of cornea is indicative of LSCD; or alternatively
a) detecting the expression of the MUC5AC gene in a sample cornea from said subject; and
b) comparing the expression level of the MUC5AC gene detected in step a) with the expression level of the MUC5AC gene in a reference sample;

wherein step a) comprises detecting the expression of the MUC5AC gene in a cornea sample from said subject, using a pair of oligonucleotide primers under conditions which allow specifically amplifying a fragment of the MUC5AC transcript, said fragment having at least 75 nucleotides long and being included within a region consisting of nucleotides 17440-18750 in the MUC5AC cDNA nucleotide sequence shown in SEQ ID NO: 1; and wherein an increase in the expression level of the MUC5AC gene detected in step a) with respect to the expression level of the MUC5AC gene in the reference sample is indicative of LSCD.

In another aspect, the invention relates to an in vitro method for diagnosing LSCD in a subject based on an increased expression of the MUC5AC gene in a cornea sample from said subject with respect to the expression level of said gene in a reference sample. In a particular embodiment, the expression of the MUC5AC gene is detected by means of RT-PCR using a pair of oligonucleotide primers consisting of the oligonucleotide primer MUC5AC-RT-F2 comprising the nucleotide sequence shown in SEQ ID NO: 2 and the oligonucleotide primer MUC5AC-RT-R2 comprising the nucleotide sequence shown in SEQ ID NO: 3, under suitable conditions.

In another aspect, the invention relates to an oligonucleotide selected from an oligonucleotide comprising the nucleotide sequence shown in SEQ ID NO: 2, an oligonucleotide comprising the nucleotide sequence shown in SEQ ID NO: 3 and combinations of both oligonucleotides.

In another aspect, the invention relates to a kit comprising at least one oligonucleotide selected from an oligonucleotide comprising the nucleotide sequence shown in SEQ ID NO: 2, an oligonucleotide comprising the nucleotide sequence shown in SEQ ID NO: 3 and combinations thereof; or, alternatively, the pair of oligonucleotide primers consisting of the oligonucleotide primer MUC5AC-RT-F2 comprising the nucleotide sequence shown in SEQ ID NO: 2 and the oligonucleotide primer MUC5AC-RT-R2 comprising the nucleotide sequence shown in SEQ ID NO: 3; said kit can be used for the diagnosis of LSCD in a subject, therefore the use of said kit for diagnosing LSCD is an additional aspect of the invention.

In another aspect, the invention relates to a method for evaluating if a patient with a corneal pathology is a suitable candidate for corneal transplantation or for a keratoplasty.

### BRIEF DESCRIPTION OF DRAWINGS

FIG. 1 is a plot comparing two sequences; in this case, the two sequences correspond to the same sequence: cDNA of MUC5AC. The dots represent sequence homology and the white areas, divergence. The X axis represents the total length of the first sequence input and the Y axis represents the total length of the second sequence input.

FIG. 2 is a plot comparing the cDNA of MUC5AC with its reverse complement, i.e. mRNA of MUC5AC. The rectangle areas are the regions with the highest content of palindromic sequences.

FIG. 3 shows the MUC5AC and MUC5B gene sequences in the genomic context, indicating that both mucins are two different forms of alternative splicing and transcription initiation coming from the same locus.

FIG. 4A shows a plot comparing the cDNA of MUC5AC and MUC5B sequences. The rectangles in the upper left and lower right corners of the plot represent the regions with the lowest degree of homology between both mucins. FIG. 4B represents a detailed view of the lower right corner of FIG. 4A (shorter sequences are compared) to gain resolution.

FIG. 5 represents the analysis of secondary structure of the MUC5AC RNA of the fragment amplified (103 base pairs (bp)) by using the oligonucleotide primers pair consisting of primer MUC5AC-RT-F2 (SEQ ID NO: 2) and primer MUC5AC-RT-R2 (SEQ ID NO: 3), showing the probability of annealing between the different regions, regions with color intensity closer to 1 are the ones with higher probability of annealing between base pairs and the ones with the color intensity closer to 0 are the one with less probability.

FIG. 6 is a photograph of an electrophoresis gel showing the result obtained for two different patients in cornea and conjunctiva samples, amplifying the MUC5AC transcript and the ACTB and GAPDH transcripts (constitutive genes) with the pair of oligonucleotide primers MUC5AC-RT-F2 (SEQ ID NO: 2) and MUC5AC-RT-R2 (SEQ ID NO: 3). Lanes 1 and 12: molecular weight marker; lane 2: MUC5AC, patient 1, cornea, negative sample; lane 3: MUC5AC, patient 1, conjunctiva, positive; lane 4: MUC5AC, patient 2, cornea, weak positive; lane 5: MUC5AC, patient 2, conjunctiva, positive control; lane 6: GAPDH, patient 1, cornea; lane 7: GAPDH, patient 1, conjunctiva; lane 8: GAPDH, patient 2, cornea; lane 9: GAPDH, patient 2, conjunctiva; lane 10: RT-H2O, negative control for retrotranscription; lane 11: PCR-H2O, negative control for PCR.

FIG. 7 is a photograph of an electrophoresis gel showing the result obtained for two different patients in cornea and conjunctiva samples, amplifying the MUC5AC transcript and the ACTB and GAPDH transcripts (constitutive genes) with the oligonucleotides primers MUC5AC-RT-F2 (SEQ ID NO: 2) and MUC5AC-RT-R2 (SEQ ID NO: 3). Lanes 1 and 12: molecular weight marker; lane 2: MUC5AC, patient 1, cornea, clear positive sample; lane 3: MUC5AC, patient 1, conjunctiva, positive control; lane 4: GAPDH, patient 1, cornea; lane 5: GAPDH, patient 1, conjunctiva; lane 6: MUC5AC, patient 2, cornea, negative; lane 7: MUC5AC, patient 2, conjunctiva, positive control; lane 8: GAPDH, patient 2, cornea; lane 9: GAPDH, patient 2, conjunctiva; lane 10: RT-H2O, negative control for retrotranscription; lane 11: PCR-H2O, negative control for PCR.

FIG. 8 shows the alignment of the sequence obtained from sequencing the amplified sequence with MUC5AC-RT-F2 (SEQ ID NO: 2) and MUC5AC-RT-R2 (SEQ ID NO: 3), of both oligonucleotides primers and of the probe #71 (Roche).

FIG. 9 shows the localization of the amplified fragment with the MUC5AC-RT-F2 (SEQ ID NO: 2) and MUC5AC-RT-R2 (SEQ ID NO: 3) primers within the genomic sequence of MUC5AC gene, with three zoom regions to render it clearer.

FIG. 10 is a photograph of an electrophoresis gel showing the results of the analysis of cornea samples from patients 5, 6 and 7 by means of RT-PCR and subsequent electrophoresis in 2.5% agarose gel and developing with ethidium bromide; lane 5 shows a band corresponding to the specific 103 base pair (bp) fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the cornea of patient 5. Lanes 1 and 8: Molecular weight markers; Lane 2: Positive reference sample of healthy conjunctiva; Lanes 3, 4 and 5: Cornea samples from patients 7, 6 and 5; Lane 6: Negative reference sample of healthy cornea; and Lane 7: Negative control of filter without biological sample.

FIG. 11 comprises a set of photographs showing a diagnosis of LSCD by means of a slit lamp examination (FIGS. 11A and 11B), impression cytology and PAS-hematoxylin staining (FIGS. 11C and 11D) and by means of RT-PCR (FIGS. 11E and 11F) of patient 0216. FIG. 11C is a microphotograph of corneal cells from the left eye (LE) of patient 0216 at a magnification of 20 (20×), wherein goblet cells can be seen. FIG. 11D is a microphotograph of corneal cells from the right eye (RE) of the same patient (patient 0216) at 20×, wherein the presence of goblet cells can also be seen. FIG. 11E is a graph showing the amplification curves obtained by means of RT-PCR of the specific fragment of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea and conjunctiva samples from the left eye (LE) and right eye (RE) of patient 0216; LSCD: Limbal stem cell deficiency. FIG. 11F is a photograph of an electrophoresis gel showing the results of the analysis of cornea samples from the left and right eyes of patient 0216 by means of RT-PCR and subsequent electrophoresis in 2.5% agarose gel and developing with ethidium bromide; lanes 2 and 4 show respective bands corresponding to the specific 103 bp fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the corneas of both eyes of patient 0216. Lanes 1 and 9: Molecular weight markers (MWM); Lane 2 (C-LE): Cornea sample from the left eye of patient 0216; Lane 3 (CJ-LE): Conjunctiva sample from the left eye of patient 0216; Lane 4 (C-RE): Cornea sample from the right eye of patient 0216; Lane 5 (CJ-RE): Conjunctiva sample from the right eye of patient 0216; and Lanes 6, 7, 8: Used as negative controls of the amplification reaction.

FIG. 12 comprises a set of photographs showing a clinical diagnosis of LSCD by impression cytology and PAS-hematoxylin staining (FIG. 12A) and by means of RT-PCR (FIGS. 12B and 12C) of patient 076. FIG. 12A is a microphotograph of corneal cells of patient 076 at 20×; the arrow indicates goblet cells. FIG. 12B is a graph showing the amplification curves obtained by means of RT-PCR of the specific fragment of the MUC5AC transcript amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea (PTE-C) and conjunctiva (PTE-CJ) samples from patient 076 and in a conjunctiva sample used as a positive control reference (CTcj). FIG. 12C is a photograph of an electrophoresis gel showing the results of the analysis of cornea and conjunctiva samples from patient 076 by means of RT-PCR and subsequent electrophoresis in 2.5% agarose gel and developing with ethidium bromide; lane 3 shows a band corresponding to the specific 103 bp fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the analyzed cornea of patient 076. Lane 1 (MWM): Molecular weight marker; Lane 2 (PTE-CJ): Conjunctiva sample from patient 076; Lane 3 (PTE-C): Cornea sample from patient 076; Lane 4 (CTcj): Conjunctiva sample used as a positive control reference; Lane 5 (CTc): Cornea sample used as a negative control reference; and Lanes 6, 7, 8: Used as negative controls of the amplification reaction.

FIG. 13 comprises a set of photographs showing a clinical diagnosis of LSCD of patient 0121 which was not confirmed by impression cytology and PAS-hematoxylin staining (FIG. 13A) or by means of RT-PCR (FIGS. 13B and 13C). FIG. 13A is a microphotograph of corneal cells of patient 0121 at 20×. FIG. 13B is a graph showing the amplification curves obtained by means of RT-PCR of the specific fragment of the MUC5AC transcript amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea (PTE-C) and conjunctiva (PTE-CJ) samples from patient 0121. FIG. 13C is a photograph of an electrophoresis gel showing the results of the analysis of cornea and conjunctiva samples from patient 0121 by means of RT-PCR and subsequent electrophoresis in 2.5% agarose gel and developing with ethidium bromide, wherein the band corresponding to the specific 103 bp fragment of the mRNA of the MUC5AC gene in the lane corresponding to the cornea sample from patient 0121 (PTE-C) is not observed, which indicates the non-expression of the MUC5AC gene in the analyzed cornea of patient 0121. Lanes 1 (MWM) and 7: Molecular weight markers; Lane 2 (PTE-C): Cornea sample from patient 0121; Lane 3 (PTE-CJ): Conjunctiva sample from patient 0121; and Lanes 5 and 6: Uses as negative controls of the amplification reaction.

FIG. 14 comprises a set of photographs showing a histological diagnosis of LSCD by impression cytology and PAS-hematoxylin staining (FIG. 14A) and by means of RT-PCR (FIGS. 14B and 14C) of patient 0123. FIG. 14A is a microphotograph of corneal cells of patient 0123 at 20×; the arrow indicates a goblet cell in the analyzed cornea. FIG. 14B is a graph showing the amplification curves obtained by means of RT-PCR of the specific fragment of the MUC5AC transcript amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea (PTE-C) and conjunctiva (PTE-CJ) samples from patient 0123. FIG. 14C is a photograph of an electrophoresis gel showing the results of the analysis of cornea and conjunctiva samples from patient 0123 by means of RT-PCR and subsequent electrophoresis in 2.5% agarose gel and developing with ethidium bromide; lane 4 shows a band corresponding to the specific 103 bp fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the analyzed cornea of patient 0123. Lane 1 (MWM): Molecular weight marker; Lanes 2 and 3: Used as negative controls of the amplification reaction; Lane 4 (PTE-C): Cornea sample from patient 0123; and Lane 5 (PTE-CJ): Conjunctiva sample from patient 0123.

FIG. 15 comprises a set of photographs showing a clinical diagnosis of LSCD of patient 0125. Although goblet cells are not seen in the cornea analyzed by impression cytology and PAS-hematoxylin staining (FIG. 15A), the presence of the specific 103 bp fragment of the mRNA of the MUC5AC gene in the cornea analyzed by RT-PCR confirms the LSCD (FIG. 15C). FIG. 15A is a microphotograph of corneal cells of patient 0125 at 20×. FIG. 15B is a graph showing the amplification curves obtained by means of RT-PCR of the specific fragment of the MUC5AC transcript amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea (PTE-C) and conjunctiva (PTE-CJ) samples from patient 0125. FIG. 15C is a photograph of an electrophoresis gel showing the results of the analysis of cornea and conjunctiva samples from patient 0125 by means of RT-PCR and subsequent electrophoresis in 2.5% agarose gel and developing with ethidium bromide; lane 5 shows a band corresponding to the specific 103 bp fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the analyzed cornea of patient 0125. Lane 1 (MWM): Molecular weight marker; Lanes 2, 3 and 4: Used as negative controls of the amplification reaction; Lane 5 (PTE-C): Cornea sample from patient 0125; and Lane 6 (PTE-CJ): Conjunctiva sample from patient 0125.

FIG. 16 comprises a set of photographs showing a clinical diagnosis of LSCD of patient 0132. Although goblet cells are not seen in the cornea analyzed by impression cytology and PAS-hematoxylin staining (FIG. 16A), the presence of the specific 103 bp fragment of the mRNA of the MUC5AC gene in the cornea analyzed by RT-PCR confirms the LSCD (FIG. 16C). FIG. 16A is a microphotograph of corneal cells of patient 0132 at 20×. FIG. 16B is a graph showing the amplification curves obtained by means of RT-PCR of the specific fragment of the MUC5AC mRNA amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea (PTE-C) and conjunctiva (CJ(+)) samples from patient 0132. FIG. 16C is a photograph of an electrophoresis gel showing the results of the analysis of cornea and conjunctiva samples from patient 0132 by means of RT-PCR and subsequent electrophoresis in 2.5% agarose gel and developing with ethidium bromide; lane 3 shows a band corresponding to the specific 103 bp fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the analyzed cornea of patient 0132. Lane 1 (MWM): Molecular weight marker; Lane 2 (CJ(+)): Conjunctiva sample from patient 0132; Lane 3 (PTE-C): Cornea sample from patient 0132; and Lanes 4, 5 and 6: Used as negative controls of the amplification reaction.

FIG. 17 comprises a set of photographs showing a clinical diagnosis of LSCD of patient 0186. Although goblet cells are not seen in the corneas of patients 0172 and 0186 analyzed by impression cytology and PAS-hematoxylin staining (FIG. 17A (patient 0172) and FIG. 17B (patient 0186)), the presence of the specific 103 bp fragment of the mRNA of the MUC5AC gene in the cornea of patient 0186 analyzed by RT-PCR (FIG. 17D) confirms the LSCD in patient 0186. FIGS. 17A and 17B are microphotographs of corneal cells (20×) of patients 0172 and 0186, respectively. FIG. 17C is a graph showing the amplification curves obtained by means of RT-PCR of the specific fragment of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea samples (PTE-C) from patient 0186 and conjunctiva samples (PTE-CJ) from patients 0186 and 0172. FIG. 17D is a photograph of an electrophoresis gel showing the results of the analysis of cornea and conjunctiva samples from patients 0172 and 0186 by means of RT-PCR and subsequent electrophoresis in 2.5% agarose gel and developing with ethidium bromide; lane 3 shows a band corresponding to the specific 103 bp fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the analyzed cornea of patient 0186; said band is not observed in lane 2 corresponding to the analyzed cornea of patient 0172. Lane 1 (MWM): Molecular weight marker; Lane 2 (PTE-C 0172): Cornea sample from patient 0172; Lane 3 (PTE-C 0186): Cornea sample from patient 0186; Lane 4 (PTE-CJ 0172): Conjunctiva sample from patient 0172; Lane 5 (PTE-CJ 0186): Conjunctiva sample from patient 0186; and Lanes 6, 7 and 8: Used as negative controls of the amplification reaction.

FIG. 18 comprises a set of photographs showing a clinical diagnosis of LSCD of patient 0214. Although goblet cells are not seen in the cornea analyzed by impression cytology and PAS-hematoxylin staining (FIG. 18A), the presence of the specific 103 bp fragment of the mRNA of the MUC5AC gene in the cornea analyzed by RT-PCR confirms the LSCD (FIG. 18C). FIG. 18A is a microphotograph of corneal cells of patient 0214 at 20×. FIG. 18B is a graph showing the amplification curves obtained by means of RT-PCR of the specific fragment of the MUC5AC transcript amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea (PTE-C) and conjunctiva (PTE-CJ) samples from patient 0214. FIG. 18C is a photograph of an electrophoresis gel showing the results of the analysis of cornea and conjunctiva samples from patient 0214 by means of RT-PCR and subsequent electrophoresis in 2.5% agarose gel and developing with ethidium bromide; lane 2 shows a band corresponding to the specific 103 bp fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the analyzed cornea of patient 0214. Lanes 1 (MWM) and 7: Molecular weight markers; Lane 2 (PCT-C): Cornea sample from patient 0214; Lane 3 (PCT-CJ): Conjunctiva sample from patient 0214; and Lanes 4, 5 and 6: Used as negative controls of the amplification reaction.

FIG. 19 comprises a set of photographs showing a clinical diagnosis of LSCD by impression cytology and PAS-hematoxylin staining (FIG. 19A) and by means of RT-PCR (FIGS. 19B and 19C) of patient 0233. FIG. 19A is a microphotograph of corneal cells of patient 0233 at 20×. FIG. 19B is a graph showing the amplification curves obtained by means of RT-PCR of the specific fragment of the MUC5AC mRNA amplified with the pair of oligonucleotide primers formed by the oligonucleotides MUC5AC-RT-F2 (SEQ ID NO: 2) and MUC5AC-RT-R2 (SEQ ID NO: 3) and using a specific fluorescent probe [sequence 5′-CTGGCTGC-3′, labeled with 6-FAM at the 5′ end] in cornea (PTE-C) and conjunctiva (PTE-CJ) samples from patient 0233. FIG. 19C is a photograph of an electrophoresis gel showing the results of the analysis of cornea and conjunctiva samples from patient 0233 by means of RT-PCR and subsequent electrophoresis in 2.5% agarose gel and developing with ethidium bromide; lane 2 shows a band corresponding to the specific 103 bp fragment of the mRNA of the MUC5AC gene amplified with the pair of oligonucleotide primers formed by the oligonucleotides MUC5AC-RT-F2 (SEQ ID NO: 2) and MUC5AC-RT-R2 (SEQ ID NO: 3), representative of the expression of the MUC5AC gene in the analyzed cornea of patient 0233. Lanes 1 and 7: Molecular weight markers; Lane 2 (PTE-C): Cornea sample from patient 0233; Lane 3 (PTE-CJ): Conjunctiva sample from patient 0233; and Lanes 4, 5 and 6: Used as negative controls of the amplification reaction.

FIG. 20 is a photograph of an electrophoresis gel showing the results of the analysis of cornea and conjunctiva samples from different patients by means of RT-PCR and subsequent electrophoresis in 2.5% agarose gel and developed with ethidium bromide. (A) corresponds to patients 3B, 4A, 4B, 5A, 7B, 6B, 10A, 11B, 12A, 13A, 13B, 14B, 19B; (B) corresponds to patients 9B, 17A, 18A and 22B; (C) corresponds to patients 6A, 7A and 8A; (D) corresponds to patients 11A, 16A, 16B, 21A, 21B, 100A, 100B, 101A, 102A and 23B; (E) corresponds to patients 1, 2A, 2B, 4A, 4B, 5A, 5B, 6A and 6B; (F) corresponds to patients AS-1, LG-2, OC3, MA4 and AAS; (G) corresponds to patients PTE 14, PTE 12 (right (R) and left (L) eye) and PTE 13; (H) corresponds to patients PTE 22, 23 and 24, (I) corresponds to patient PTE 820074. C is the cornea sample and CJ the conjunctiva sample. RT-H2O is the negative control for the RT-PCR and PCR-H2O is the negative control for the PCR. M is the molecular weight marker.

### DETAILED DESCRIPTION OF THE INVENTION

Limbal stem cell deficiency (LSCD) is characterized by the existence of conjunctivalization with the presence of goblet cells on the cornea; given that there are no goblet cells in the cornea, the detection of the MUC5AC gene transcript in a cornea sample from a subject is indicative of the presence of goblet cells in the cornea and, therefore, of said subject suffering from LSCD.

### Diagnosis of Limbal Stem Cell Deficiency (LSCD)

In an aspect, the invention relates to an in vitro method, hereinafter “method of the invention”, for diagnosing limbal stem cell deficiency (LSCD) in a subject, comprising:
analyzing the expression of the MUC5AC gene in a sample of cornea from said subject, using a pair of oligonucleotide primers under conditions which allow specifically amplifying a fragment of the MUC5AC transcript, said fragment having at least 75 nucleotides long and being included within a region consisting of nucleotides 17440-18750 in the MUC5AC cDNA nucleotide sequence shown in SEQ ID NO: 1, wherein the detection of the expression of the MUC5AC gene in said sample of cornea is indicative of LSCD; or alternatively
a) detecting the expression of the MUC5AC gene in a sample cornea from said subject; and
b) comparing the expression level of the MUC5AC gene detected in step a) with the expression level of the MUC5AC gene in a reference sample;

wherein step a) comprises detecting the expression of the MUC5AC gene in a cornea sample from said subject, using a pair of oligonucleotide primers under conditions which allow specifically amplifying a fragment of the MUC5AC transcript, said fragment having at least 75 nucleotides long and being included within a region consisting of nucleotides 17440-18750 in the MUC5AC cDNA nucleotide sequence shown in SEQ ID NO: 1; and

wherein an increase in the expression level of the MUC5AC gene detected in step a) with respect to the expression level of the MUC5AC gene in the reference sample is indicative of LSCD.

In a particular embodiment, the method of the invention comprises analyzing the expression of the MUC5AC gene in a sample of cornea from a subject, in order to detect the expression of MUC5AC in said sample, using a pair of oligonucleotide primers under conditions which allow specifically amplifying a fragment of the MUC5AC transcript, said fragment having at least 75 nucleotides long and being included within a region consisting of nucleotides 17440-18750 in the MUC5AC cDNA nucleotide sequence shown in SEQ ID NO: 1, wherein the detection of the expression of the MUC5AC gene in said sample of cornea is indicative of LSCD.

The expression of the MUC5AC gene can be detected by any conventional method suitable for detecting the expression of genes; for example, methods comprising the reverse transcription (RT) of the gene of interest (MUC5AC) and the enzymatic amplification of a specific fragment of said gene of interest (MUC5AC).

According to the invention, said specific fragment of the MUC5AC gene which is amplified by the method of the invention has at least 75, or 100, or 200, or 250, or 300, or 500, or 750, or 1000, or 1300, or up to 1311 nucleotides long and is included within the region consisting of nucleotides 17440-18750 (1311 bp long) in the nucleotide sequence shown in SEQ ID NO: 1. Thus, the amplified fragment length is comprised between 75 and 1311 nucleotides. The full length of the amplified fragment is included within the region consisting of nucleotides 17440-18750 of SEQ ID NO: 1.

In a particular and preferred embodiment, said specific fragment to be specifically amplified by the oligonucleotide primers is the fragment consisting of 103 bp comprised between nucleotides 17749 and 17851 of the MUC5AC cDNA (SEQ ID NO: 1). Said fragment is present in the retrotranscribed cDNA of the MUC5AC gene but not in the cDNA sequences of other mucin genes, for example, MUC5B gene. Said specific 103 bp fragment can be specifically amplified by a suitable amplification reaction, e.g., by means of a polymerase chain reaction (PCR) in any suitable variant thereof, by using a pair of oligonucleotide primers consisting of an oligonucleotide primer comprising the nucleotide sequence identified as SEQ ID NO: 2 (MUC5AC-RT-F2) and an oligonucleotide primer comprising the nucleotide sequence identified as SEQ ID NO: 3 (MUC5AC-RT-R2) under conditions which allow amplifying said 103 bp fragment of the transcript of the MUC5AC gene.

In another particular embodiment, the fragment to be amplified by the oligonucleotide primers is a fragment (i) having at least 75 nucleotides long, (ii) being included within a region consisting of nucleotides 17440-18750 in the MUC5AC cDNA nucleotide sequence shown in SEQ ID NO: 1, and (iii) comprising the 103 bp comprised between nucleotides 17749 and 17851 of the MUC5AC cDNA (SEQ ID NO: 1).

Although virtually any method allowing the enzymatic amplification of a specific fragment of the MUC5AC gene can be used to put the method of the invention into practice, in a particular embodiment the enzymatic amplification of a specific fragment of the MUC5AC gene is carried out by means of the polymerase chain reaction (PCR), in any of its variants. The protocol followed to carry out a PCR in any of its variants is widely known in the state of the art and currently there are commercial kits containing the materials necessary for carrying out said amplification. The expression of the specific amplified fragment of the MUC5AC gene can be detected by means of any conventional method known by the skilled person in the art of detecting nucleic acids, for example, by means of separation (e.g., by agarose gel electrophoresis, etc.) and developing of the amplification product (e.g., by ethidium bromide staining, etc.).

In order to put it into practice the method of the invention according to this particular embodiment, a sample of cornea from the subject under study (test sample) is obtained. The cornea sample (test sample) can be obtained by conventional methods known by the persons skilled in the art, for example, by means of impression cytology (see Example 1.2). In a particular embodiment, said cornea sample (test sample) comprises corneal epithelium tissue. Further, in a specific embodiment, once the cornea sample from the subject to be analyzed is obtained, the ribonucleic acid (RNA) contained in said sample is extracted. The extraction of the RNA can be carried out by means of any conventional technique known by the persons skilled in the art using, if desired, commercial kits and reagents, e.g., the RNeasy PLUS Micro kit (Qiagen, P/N: 74034). From the extracted RNA, the complementary DNA (cDNA) in relation to the RNA corresponding to the MUC5AC gene is synthesized by conventional methods, for example, by means of reverse transcription or retro-transcription (RT) and subsequently a specific fragment of the MUC5AC reversely transcribed transcript is enzymatically amplified by any suitable technique, for example by means of classic or conventional PCR, or by any of its variants, by using the suitable oligonucleotide primers.

The suitable oligonucleotide primers are primers which specifically amplify a fragment that has at least 75, or 100, or 200, or 250, or 300, or 500, or 750, or 1000, or 1300, or up to 1311 nucleotides long and is included within the region consisting of nucleotides 17440-18750 (1311 bp long) in the nucleotide sequence shown in SEQ ID NO: 1. Thus, the amplified fragment length is comprised between 75 and 1311 nucleotides. The full length of the amplified fragment is included within the region consisting of nucleotides 17440-18750 (SEQ ID NO: 1).

In a particular and preferred embodiment, the primers are oligonucleotide primers suitable for amplifying a fragment comprising or consisting of the 103 bp comprised between nucleotides 17749 and 17851 of the MUC5AC cDNA (SEQ ID NO: 1). Virtually any pair of oligonucleotide primers suitable for amplifying said fragment can be used for performing the method of the invention. In a particular embodiment, the pair of oligonucleotide primers suitable for amplifying said 103 bp fragment consists of an oligonucleotide primer comprising the nucleotide sequence identified as SEQ ID NO: 2 (MUC5AC-RT-F2) and an oligonucleotide primer comprising the nucleotide sequence identified as SEQ ID NO: 3 (MUC5AC-RT-R2). Said pair of oligonucleotide primers [i.e., oligonucleotides comprising the nucleotide sequences shown in SEQ ID NO: 2 (MUC5AC-RT-F2) and in SEQ ID NO: 3 (MUC5AC-RT-R2)] is very important for the high specificity of the method of the invention since said primers exclusively amplify in a specific manner retro-transcribed cDNA of MUC5AC (in no case MUC5B). The oligonucleotides comprising the nucleotide sequences shown in SEQ ID NO: 2 (MUC5AC-RT-F2) and in SEQ ID NO: 3 (MUC5AC-RT-R2) and the combinations thereof, as well as the use thereof in the diagnosis of LSCD are an additional aspect of this invention as described in detail below.

In another particular embodiment, it is selected a pair of oligonucleotide primers suitable for amplifying a fragment said fragment (i) having at least 75 nucleotides long, (ii) being included within a region consisting of nucleotides 17440-18750 in the MUC5AC cDNA nucleotide sequence shown in SEQ ID NO: 1, and (iii) comprising the 103 bp comprised between nucleotides 17749 and 17851 of the MUC5AC cDNA (SEQ ID NO: 1). Virtually any pair of oligonucleotide primers suitable for amplifying said fragment having the previously mentioned features can be used in performing the method of the invention.

The protocol followed to carry out PCR is widely known in the state of the art and currently there are commercial kits containing the materials necessary for carrying out said amplification. Likewise, the conditions of temperature, time, concentrations of reagents and number of PCR cycles will depend on the DNA polymerase used in the amplification reaction, on the specificity, length and composition of the primers, and the length and composition of the sequence. If a commercial kit is used, the conditions of the reaction will be those specified by the manufacturer of the kit.

The detection of the MUC5AC gene in a cornea sample from a subject is indicative of the presence of goblet cells in the cornea, and, consequently, of said subject suffering from LSCD.

The method of the invention has a number of advantages since it is a non-invasive method, with high sensitivity and specificity (since the fragment amplified comprises a concrete and specific region of the MUC5AC gene), high reproducibility and, furthermore, enables the identification of erroneous clinical diagnoses. The RNA transcript of the MUC5AC gene is intrinsically very difficult to amplify, because it contains several intrinsic features which render it complicated: (1) there are too many nucleotide repeats, (2) the content in G/C is high, and (3) the secondary structure of the RNA to be amplified is such that it anneals with itself. The sequence to be amplified in the method of the invention overcomes these problems of the transcript. In relation with specificity, the main problem is that the degree of homology between several members of the MUC family is very high. The method of the invention also overcomes this problem, because the amplified fragment in the method of the invention is uniquely specific for MUC5AC gene and is not present in the MUC5B gene, which is highly homolog to MUC5AC. The fragment to be amplified in the MUC5AC transcript, which is within a good region because it overcomes all the issues described above, is comprised between nucleotides 17440-18750 of the MUC5AC cDNA sequence (SEQ ID NO: 1).

In another particular embodiment, the method of the invention comprises:
a) detecting the expression of the MUC5AC gene in a sample cornea from said subject; and
b) comparing the expression level of the MUC5AC gene detected in step a) with the expression level of the MUC5AC gene in a reference sample;

wherein step a) comprises detecting the expression of the MUC5AC gene in a cornea sample from said subject, using a pair of oligonucleotide primers under conditions which allow specifically amplifying a fragment of the MUC5AC transcript, said fragment having at least 75 nucleotides long and being included within a region consisting of nucleotides 17440-18750 in the MUC5AC cDNA nucleotide sequence shown in SEQ ID NO: 1; and wherein an increase in the expression level of the MUC5AC gene detected in step a) with respect to the expression level of the MUC5AC gene in the reference sample is indicative of LSCD.

In order to put it into practice the method of the invention according to this particular embodiment, a sample of cornea from the subject under study (test sample) is obtained. The reference sample is a sample of a tissue which does not express the MUC5AC gene, for example, corneal epithelial tissue, since healthy cornea lacks goblet cells and, therefore, there is no expression of the MUC5AC gene; consequently, said reference sample acts as a negative control of expression of the MUC5AC gene. Additionally, if desired, a sample of tissue expressing the MUC5AC gene, for example, conjunctival epithelial tissue since the conjunctiva contains goblet cells expressing the MUC5AC gene, can be incorporated as a positive control. The cornea samples (test sample and reference sample), like the conjunctiva sample (positive control of expression of the MUC5AC gene) can be obtained by conventional methods known by the persons skilled in the art, for example, by means of impression cytology (see Example 1.2). In a particular embodiment, both cornea samples (test sample and reference sample) comprise corneal epithelium tissue. Likewise, in a particular embodiment, the conjunctiva sample (in the event of being used) comprises conjunctival epithelium cells. The conjunctiva sample can be obtained by conventional methods, for example, by means of impression cytology, brush cytology or biopsy. All the samples (test, reference or negative control and, optionally, positive control of expression of the MUC5AC gene) are separately processed in the same manner.

The particulars of the fragment to be amplified as well as of the pair of oligonucleotide primers to be used have been previously mentioned in connection with the first particular embodiment (option) of the method of the invention.

The detection of the expression of the MUC5AC gene can be carried out by any suitable method for the detection of the expression of a gene; for example, by means of a method comprising the reverse transcription (RT) of the gene of interest (MUC5AC), the enzymatic amplification of a specific fragment of said gene of interest (MUC5AC) and its quantification. In a particular embodiment, the amplification and quantification of the expression of the MUC5AC gene is carried out by means of PCR; although a classic or conventional PCR can be used, in practice the use of a Reverse Transcription Real-Time-PCR (RT-PCR) or “real-time PCR” is preferred since it allows amplifying and simultaneously detecting the DNA amplification product using the suitable oligonucleotide primers and a substance labeled with a fluorophore since classic PCR requires carrying out additional steps (separation, staining and preparation of standards) which complicate the process. Any RT-PCR technique can be used, both the techniques based on unspecific fluorochromes (e.g., techniques based on using fluorochromes which bind specifically to double-stranded DNA such as SYBR Green, etc.), and the techniques based on using specific probes (e.g., Taqman type probes, Molecular Beacon type probes, Scorpion type probes, etc). Therefore, in a particular and preferred embodiment, the detection of the expression of the MUC5AC gene is carried out by means of RT-PCR; in Example 1 the detection of the expression of the MUC5AC gene is carried out by means of RT-PCR using a Taqman type probe.

In order to quantify the expression of the MUC5AC gene, in a particular embodiment, the RNA contained in the samples (test, reference and, optionally, positive control) is extracted. The extraction of the RNA can be carried out by means of any technique allowing the extraction of RNA, for example, by using, if desired, commercial kits and reagents, e.g., the RNeasy PLUS Micro kit (Qiagen, P/N: 74034). From the extracted RNA, the complementary DNA (cDNA) in relation to the RNA corresponding to the MUC5AC gene is synthesized by conventional methods, for example, by means of RT using the suitable reagents and conditions, and, subsequently, the enzymatic amplification of a specific fragment of the MUC5AC gene and the simultaneous quantification of the expression of said MUC5AC gene are carried out using the suitable oligonucleotide primers, dNTPs, a suitable reaction buffer and a heat-stable DNA polymerase, and a substance labeled with a fluorophore, e.g., a Taqman type probe is incorporated to the reaction mixture formed. As mentioned above, in a particular and preferred embodiment, the enzymatic amplification of a specific fragment of the MUC5AC gene and its simultaneous quantification is carried out by means of a RT-PCR using a pair of oligonucleotide primers consisting of an oligonucleotide primer comprising the nucleotide sequence identified as SEQ ID NO: 2 (MUC5AC-RT-F2) and an oligonucleotide primer comprising the nucleotide sequence identified as SEQ ID NO: 3 (MUC5AC-RT-R2) [the use of this pair of oligonucleotides allows specifically amplifying a fragment of retrotranscribed cDNA of MUC5AC] under conditions which allow amplifying a specific 103 base pair (bp) fragment of the transcript of the MUC5AC gene, in the presence of a substance labeled with a fluorophore, such as a fluorochrome which binds specifically to double-stranded DNA (e.g., SYBR Green, etc.), or a specific probe (e.g., a Taqman type, or Molecular Beacon type, or Scorpion type probe, etc); in a particular embodiment, the quantification of the expression of the MUC5AC gene is carried out by means of RT-PCR using a specific Taqman type probe which has the nucleotide sequence 5′-CTGGCTGC-3′ and is labeled at the 5′ end with the fluorophore 6-FAM (6-carboxyfluorescein), compatible for the detection thereof in real-time RT-PCR equipment (Example 1.3).

The use of the pair of oligonucleotides consisting of an oligonucleotide comprising the nucleotide sequence shown in SEQ ID NO: 2 (MUC5AC-RT-F2) and an oligonucleotide comprising the nucleotide sequence shown in SEQ ID NO: 3 (MUC5AC-RT-R2) is very important for achieving the high specificity of the method of the invention since said primers exclusively amplify in a specific manner retrotranscribed cDNA of MUC5AC (in no case MUC5B).

In another particular embodiment, it is selected a pair of oligonucleotide primers suitable for amplifying a fragment said fragment (i) having at least 75 nucleotides long, (ii) being included within a region consisting of nucleotides 17440-18750 in the MUC5AC cDNA nucleotide sequence shown in SEQ ID NO: 1, and (iii) comprising the 103 bp comprised between nucleotides 17749 and 17851 of the MUC5AC cDNA (SEQ ID NO: 1). Virtually any pair of oligonucleotide primers suitable for amplifying said fragment having the previously mentioned features can be used in performing the method of the invention.

As discussed above, the protocol followed to carry out the RT-PCR is widely known in the state of the art and currently there are commercial kits containing the materials necessary for carrying out said amplification. Likewise, the conditions of temperature, time, reagents, concentrations of reagents and number of PCR cycles will depend on the DNA polymerase used in the amplification reaction, on the specificity, length and composition of the primers, and the length and composition of the sequence. If a commercial kit is used, the conditions of the reaction will be those specified by the manufacturer of the kit. The reagents and the conditions used to put the method of the invention into practice are shown in Example 1 attached to this description. In a particular embodiment, the method of the invention is carried out using the reaction mixture described in Example 1; nevertheless, any other suitable combination of reaction mixture can be used.

According to this particular embodiment, the method of the invention comprises, in addition to quantifying the expression of the MUC5AC gene in a cornea sample from the subject that is subjected to analysis [step a)], comparing the expression level of the MUC5AC gene detected in step (a) with the expression level of the MUC5AC gene in a reference sample [step b)] to establish if the subject under study suffers from LSCD.

In a specific embodiment, the reference sample is a healthy cornea sample, i.e., from a subject who does not suffer from LSCD. Alternatively, the expression level of the MUC5AC gene in a reference sample can be established as a reference value and can be obtained from the values of expression of the MUC5AC gene obtained from samples of corneal epithelial tissue obtained from one or more, for example, 2 or more, 10 or more, 20 or more, etc., subjects who do not suffer from LSCD. It would thus not be necessary to obtain a reference cornea sample or to quantify the expression of the MUC5AC gene in said reference sample every time an assay for diagnosing LSCD is carried out, which would simplify the work and save costs.

Once the expression level of the MUC5AC gene in the sample from the subject to be analyzed has been obtained and the expression level of the MUC5AC gene in a reference sample or the reference value of the expression of said MUC5AC gene in a reference sample has been determined, the expression level of the MUC5AC gene detected in the test sample is compared with the expression level of the MUC5AC gene in the reference sample or with the reference value to determine if has or has not been a change in the expression level of the MUC5AC gene detected in said test sample with respect to the expression level of the MUC5AC gene in the reference sample or with respect to the reference value. An increase in the expression of said MUC5AC gene in the test sample under study is considered as a “significant increase” with respect to the expression level of the MUC5AC gene in the reference sample or with respect to the reference value when the expression level in the test sample increases with respect to the expression level of the MUC5AC gene in the reference sample or with respect to the reference value by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, or even more. As it has been previously mentioned, an increase in the expression level of the MUC5AC gene in a cornea sample from the subject under study with respect to the expression level of the MUC5AC gene in the reference sample or with respect to the reference value is indicative of said subject suffering from LSCD.

In a particular embodiment of the method of the invention, the invention provides an in vitro method for diagnosing LSCD in a subject, comprising analyzing the expression of the MUC5AC gene in a cornea sample from said subject, by an amplification reaction, using a pair of oligonucleotide primers consisting of an oligonucleotide primer comprising the nucleotide sequence identified as SEQ ID NO: 2 (MUC5AC-RT-F2) and an oligonucleotide primer comprising the nucleotide sequence identified as SEQ ID NO: 3 (MUC5AC-RT-R2), under conditions which allow amplifying a specific fragment of the MUC5AC gene, wherein the detection of the expression of the MUC5AC gene in said sample of cornea is indicative of LSCD.

As it has been previously mentioned, the fragment amplified by said pair of oligonucleotide primers comprises or consists of the 103 bp comprised between nucleotides 17749 and 17851 of the MUC5AC cDNA (SEQ ID NO: 1), said fragment comprising a specific region of the MUC5AC gene. Said fragment is amplified by an amplification reaction, for example, by an enzymatic amplification reaction, such as, for example, by PCR in any of its variants.

In another particular embodiment of the method of the invention, the invention provides an in vitro method for diagnosing LSCD in a subject, comprising:
a) detecting the expression of the MUC5AC gene in a cornea sample from said subject, by an amplification reaction, using a pair of oligonucleotide primers consisting of an oligonucleotide primer comprising the nucleotide sequence identified as SEQ ID NO: 2 (MUC5AC-RT-F2) and by an oligonucleotide primer comprising the nucleotide sequence identified as SEQ ID NO: 3 (MUC5AC-RT-R2), under conditions which allow amplifying a specific fragment of the MUC5AC gene; and
b) comparing the expression level of the MUC5AC gene detected in step (a) with the expression level of the MUC5AC gene in a reference sample;
   wherein an increase in the expression level of the MUC5AC gene detected in step (a) with respect to the expression level of the MUC5AC gene in the reference sample is indicative of LSCD.

A healthy cornea sample, i.e., from a subject who does not suffer from LSCD, can be used as a reference sample. Alternatively, the expression level of the MUC5AC gene determined in step a) can be compared with the reference value of expression of the MUC5AC gene in healthy cornea obtained from the values of expression of the MUC5AC gene obtained from samples of corneal epithelial tissue obtained from one or more subjects who do not suffer from LSCD. Additionally, if desired, the expression level of the MUC5AC gene in a sample of tissue expressing the MUC5AC gene, for example, conjunctival epithelial tissue, can be quantified and said sample can be used as a positive control of amplification of said MUC5AC gene. As disclosed above, the fragment amplified by said pair of oligonucleotide primers comprises or consists of the 103 bp comprised between nucleotides 17749 and 17851 of the MUC5AC cDNA (SEQ ID NO: 1), said fragment comprising a specific region of the MUC5AC gene. Said fragment is amplified by an amplification reaction, for example, by an enzymatic amplification reaction, such as, for example, by PCR in any of its variants.

### Oligonucleotide Primers

As it has been previously mentioned, in another aspect, the invention relates to an oligonucleotide selected from the group consisting of an oligonucleotide comprising the nucleotide sequence shown in SEQ ID NO: 2 (MUC5AC-RT-F2), an oligonucleotide comprising the nucleotide sequence shown in SEQ ID NO: 3 (MUC5AC-RT-F2) and mixtures thereof.

When said oligonucleotides are used as oligonucleotide primers in a PCR, such as a classic PCR or a variant thereof (e.g., RT-PCR), they allow amplifying a concrete and specific region or fragment of the MUC5AC transcript but not of other mucin genes (e.g., MUC5B), therefore they can be used to enzymatically amplify, for example, by means of PCR, a specific fragment of the MUC5AC transcript, or to detect and identify said MUC5AC transcript, or to quantify said MUC5AC transcript, or in the diagnosis of LSCD by means of detecting and/or quantifying the MUC5AC transcript by PCR (e.g., classic Reverse transcription PCR or Reverse transcription Real Time-PCR).

### Kits

The reagents necessary for putting the method of the invention into practice can be included in a kit. Said kit and its use in the diagnosis of LSCD are additional aspects of this invention, which will be described in detail below.

Therefore, in another aspect, the invention relates to a kit, hereinafter kit of the invention, comprising an oligonucleotide selected from the group consisting of an oligonucleotide comprising the nucleotide sequence shown in SEQ ID NO: 2 (MUC5AC-RT-F2), an oligonucleotide comprising the nucleotide sequence shown in SEQ ID NO: 3 (MUC5AC-RT-F2) and mixtures thereof.

In a particular embodiment, said kit of the invention comprises at least the pair of oligonucleotide primers formed by an oligonucleotide primer comprising SEQ ID NO: 2 (MUC5AC-RT-F2) and an oligonucleotide primer comprising SEQ ID NO: 3 (MUC5AC-RT-R2). A kit of this type is particularly useful for detecting the expression of the MUC5AC transcript by means of PCR and, consequently, said kit can be used in the diagnosis of LSCD in a subject.

In another particular embodiment, said kit of the invention furthermore comprises a substance labeled with a fluorophore, such as a fluorochrome which binds specifically to double-stranded DNA (e.g., SYBR Green, etc.), or a probe specific for the MUC5AC transcript (e.g., a Taqman type probe, a Molecular Beacon type probe, a Scorpion type probe, etc.); in a specific embodiment, said substance labeled with a fluorophore is a Taqman type probe specific for the MUC5AC transcript amplified by the oligonucleotide primers provided by this invention, such as the probe (Probe #71) (Roche) which has the nucleotide sequence 5′-CTGGCTGC-3′ and is labeled at the 5′ end with the fluorophore 6-FAM. A kit of this type is particularly useful for analyzing the expression of the MUC5AC gene or for detecting the expression of the MUC5AC gene or for quantifying the expression of the MUC5AC transcript by means of quantitative PCR and, consequently, said kit can be used in the diagnosis of LSCD in a subject.

### Selection of Suitable Patients for Corneal Transplantation or Keratoplasty

As is known, some types of blindness can be caused by an opacity or loss of the optical properties of the cornea; in some cases the remedy thereof is possible by means of corneal transplantation or keratoplasty, i.e., replacing the damaged part of the cornea with a fragment of healthy and transparent cornea from a donor.

For the keratoplasty to be successful, it is necessary for the tissue bed of the recipient to be healthy. In a conventional central keratoplasty the epithelial cells of the donor button are usually limited to 12-18 months [Kinoshita et al., Invest Ophthalmol Vis Sci (1981), 21(3):434-41]. However, if the corneal and limbal peripheral epithelium of the recipient is compromised, the performance of the keratoplasty would assure a competent epithelium only while the cells thereof survive. When the latter are replaced with the peripheral corneal epithelium of the recipient or with the conjunctival one if the limbal barrier is not competent, the typical manifestations of the disease of fundamental corneal cells would become evident with the onset of persistent epithelial defects, chronic inflammation and tendency to neovascularization which would compromise the transparency of the graft and its future transplantation.

It would therefore be convenient to have a method which allows classifying or selecting patients with a corneal pathology who can be subjected to keratoplasty with reasonable expectations of success for the purpose of reducing or minimizing the risk of failure of said technique.

The invention provides a solution to said need based on detecting and/or quantifying the MUC5AC gene in a cornea sample from said patient with a corneal pathology susceptible to keratoplasty such that, if said MUC5AC gene is detected in a cornea sample from said patient, or if the expression of said MUC5AC gene in a cornea sample from said patient is greater than a reference value of the expression of the MUC5AC gene in a reference sample (e.g., healthy cornea), then said patient suffers from LSCD and, therefore, the keratoplasty would be ineffective since it would not allow remedying said corneal pathology. In this case, said patient who suffers from LSCD should be treated by means of a suitable treatment, for example, by means of limbal stem cell transplantation. Limbal stem cell transplantation consists of partially or completely substituting the limbus of the recipient with a healthy limbus, normally an autotransplant (from the contralateral healthy eye) or an allotransplant (from a donor), the latter must be associated with systemic immunosuppression. Limbal stem cell transplantation has been proposed as an effective measure for restoring the epithelial integrity of the patient [Kenyon K R & Tseng S C. Ophthalmology (1989), 96:709-723; Thoft R A. Ophthalmology (1982), 89:335; Thoft R A. Am J Ophthalmology (1984), 97:1-6).

Therefore, in another aspect, the invention relates to a method for evaluating if a patient with a corneal pathology susceptible to keratoplasty is a suitable candidate for keratoplasty, comprising:
detecting the expression of the MUC5AC gene in a cornea sample from said patient; or, alternatively
determining the expression level of the MUC5AC gene in a cornea sample from said patient; and
   if the MUC5AC transcript is not detected in said cornea sample from said patient; or, alternatively, if the expression level of the MUC5AC gene detected in the cornea sample from said patient is equal to the expression level of the MUC5AC gene in a reference sample, then said patient is a suitable candidate for keratoplasty.

As used herein, the term “corneal pathology susceptible to keratoplasty” includes aniridia, keratitis associated with multiple endocrine deficiencies, neurotrophic keratopathy, pterygium and pseudopterygium, chemical and thermal injuries, Stevens-Johnson syndrome, multiple surgeries or cryotherapies in the limbal region, keratopathy induced by contact lenses, caustications, hypovitaminosis and herpes, among others [Tseng S C G. Eye (1989), 3:141-157; Mendicute et al. Arch Soc Esp Oftalmol (1994), 66:435-442; Barraquer et al. Atlas de microcirugia de la cornea, 1982].

In this case, the reference sample is a healthy cornea sample. The characteristics of the cornea sample from the patient and of the reference sample (healthy cornea) as well as the manner of obtaining them, and the methodology for detecting and/or quantifying the expression of the MUC5AC gene in said cornea samples have already been mentioned previously in relation to the diagnosis of LSCD. Likewise, as it has been previously mentioned in relation to the method of the invention, the quantification of the expression level in the reference sample can be replaced with a reference value of the expression of the MUC5AC gene in healthy cornea (reference value in this case) determined from the values of expression of the MUC5AC gene in samples of corneal epithelial tissue obtained from one or more, for example, 2 or more, 10 or more, 20 or more, etc., subjects who do not suffer from LSCD. It would thus not be necessary to obtain a reference cornea (healthy cornea) sample or to quantify the expression of the MUC5AC gene in said reference sample every time it is to be evaluated if a patient with a corneal pathology susceptible to keratoplasty is a suitable candidate for keratoplasty according to the present invention, which would simplify the work and save costs.

According to the previously described method, if the MUC5AC transcript is not detected in said cornea sample from said patient; or, alternatively, if the expression level of the MUC5AC gene detected in the cornea sample from said patient is equal to the expression level of the MUC5AC gene in a reference sample or to the reference value, then said patient is a suitable candidate for keratoplasty since said patient does not suffer from LSCD, which could cause the failure of the keratoplasty.

The invention is illustrated below based on the following examples which are provided by way of a non-limiting illustration of the scope of the invention.

### EXAMPLE 1

### Evaluation of the Expression of the MUC5AC Gene in the Cornea as a Marker for the Diagnosis of Limbal Stem Cell Deficiency by Means of RT-PCR

### Materials and Methods

1.1 Characterization of the MUC5AC Gene, Fragment of mRNA to be Amplified and Design of Oligonucleotide Primers

The human MUC5AC gene (NM_{—}017511) is a 112.8 kb gene, located in chromosome 11, the coordinates of which are chr11:1, 132, 474-1, 245, 302 (11p15.5). For the purpose of designing the oligonucleotide primers, the complementary reverse sequence of the RNA of the MUC5AC gene was used, since the real-time PCR is carried out on cDNA. The MUC5AC cDNA sequence corresponds to SEQ ID NO: 1.

1.1.1 Bibliographic Approach

In a preliminary approach, a bibliographic review of the most recent publications was performed to evaluate the assays being carried out by other researchers, with special interest in the molecular diagnosis of limbal deficiency.

Two problems were found during this process. Firstly, there were only available a limited number of papers about the detection of MUC5AC transcript and related to ophthalmology; only papers after 2003 were considered, the year when the first draft of the human genome sequence was published (previous papers were dropped due to the expected bad quality of the sequence data). Secondly, most of this type of articles (ophthalmology and RT-PCR MUC5AC) described assays that were based on previously published primer designs.

Under these circumstances, the inventors also considered the possibility of including papers not related with ophthalmology, but in which molecular detection of MUC5AC transcript was performed. On a molecular context, this does not constitute any difference, as the sequence of the transcript of interest (the target) is the same in any tissue considered (the quantity of the transcript may differ between tissues, but not its sequence).

Once the bibliographic review was performed, and the published primers to test were selected, the inventors tested them by means of bioinformatic methods using the Primer Blast application of NCBI (http://www.ncbi.nlm.nih.gov/tools/primer-blast/).

On the following, the results of the bioinformatic analysis are shown with the following information structure, for each scientific publication related to MUC5AC:
Primer sequences.
Name of the amplified target (gene, transcript, molecular clone, partial/total . . . ). For example: >NM_{—}001174096.1 Homo sapiens zinc finger E-box binding homeobox 1 (ZEB1), transcript variant 9, mRNA
Amplified product length.
Sequence divergence between the target showed (the most probable) and the primers tested.

Argüeso et al. 2002. Investigative Ophthalmology and Visual Science; 43: 1004-1011

| | Sequence (5′->3′) | Length | Tm | GC % |
|---|---|---|---|---|
| Forward primer | TCCACCATATACCGCCACAGA | 21 | 54.71 | 52.38% |
| Reverse primer | TGGACCGACAGTCACTGTCAAC | 22 | 56.56 | 54.55% |

>XM_002344536.1 PREDICTED: Homo sapiens similar to mucin (LOC100293983), partial mRNA
product length = 103

| | |
|---|---|
| Forward primer | 1 TCCACCATATACCGCCACAGA 21 |
| Template | 805 ..................... 825 |
| Reverse primer | 1 TGGACCGACAGTCACTGTCAAC 22 |
| Template | 907 .....G................ 886 |

Gipson et al. 2003. Invest Ophthalmol Vis Sci.; 44(6): 2496-506
Primers described in the following papers are used:
1. Finkbeiner WE, Carrier SD, Teresi CE. Am J Respir Cell Mol Biol. 1993; 9: 547-556.
2. Gipson IK, Spurr-Michaud S, Moccia R, et al. Biol Reprod. 1999; 60: 58-64.
F: TCC GAG GCC ACC TGT GAG GG
R: GAC ATC TCG GAG CAG GAA GC
PRIMER BLAST RESULTS:
Primer pair 1

| | Sequence (5′->3′) | Length | Tm | GC % |
|---|---|---|---|---|
| Forward primer | TCCGAGGCCACCTGTGAGGG | 20 | 59.90 | 70.00% |
| Reverse primer | GACATCTCGGAGCAGGAAGC | 20 | 54.81 | 60.00% |

Products on intended target
Products on allowed transcript variants
Products on potentially unintended templates
Products on target templates
>NM_001174096.1 Homo sapiens zinc finger E-box binding homeobox 1 (ZEB1), transcript variant
9, mRNA
product length = 89

| | |
|---|---|
| Forward primer | 1 TCCGAGGCCACCTGTGAGGG 20 |
| Template | 3134 ......T.GC.......... 3115 |
| Reverse primer | 1 GACATCTCGGAGCAGGAAGC 20 |
| Template | 3046 ....G.A.A..........A 3065 |

>NM_001174095.1 Homo sapiens zinc finger E-box binding homeobox 1 (ZEB1), transcript variant
8, mRNA
product length = 89

| | |
|---|---|
| Forward primer | 1 TCCGAGGCCACCTGTGAGGG 20 |
| Template | 2930 ......T.GC.......... 2911 |
| Reverse primer | 1 GACATCTCGGAGCAGGAAGC 20 |
| Template | 2842 ....G.A.A..........A 2861 |

Corrales et al. 2003. Arch Soc Esp Oftalmol.; 78(7): 375-81
Primer pair 1

| | Sequence (5′->3′) | Length | Tm | GC % |
|---|---|---|---|---|
| Forward primer | AAGTCCATGGATATCGTCCTCACT | 24 | 54.88 | 45.83% |
| Reverse primer | GTGTTGTTGTGGAAGAGGCTGTAG | 24 | 55.93 | 50.00% |

>NM_002458.2 Homo sapiens mucin 5B, oligomeric mucus/gel-forming (MUC5B), mRNA
product length = 230

| | |
|---|---|
| Forward primer | 1 AAGTCCATGGATATCGTCCTCACT 24 |
| Template | 15480 ........................ 15503 |
| Reverse primer | 1 GTGTTGTTGTGGAAGAGGCTGTAG 24 |
| Template | 15709 ........................ 15686 |

Sabo et al. 2008. Clin Cancer Res.; 14(20): 6440-8
Primer pair 1

| | Sequence (5′->3′) | Length | Tm | GC % |
|---|---|---|---|---|
| Forward primer | TCCTTCGAGTGCTTGTGTTG | 20 | 52.70 | 50.00% |
| Reverse primer | GCCTTTCAGCTACACGAGGT | 20 | 54.26 | 55.00% |

>NM_018316.1 Homo sapiens kelch-like 26 (Drosophila) (KLHL26), mRNA
product length = 108

| | |
|---|---|
| Reverse primer | 1 GCCTTTCAGCTACACGAGGT 20 |
| Template | 351 ...C.......CG.T..C.. 332 |
| Reverse primer | 1 GCCTTTCAGCTACACGAGGT 20 |
| Template | 244 .......CTGC....A.... 263 |

>NM_000820.2 Homo sapiens growth arrest-specific 6 (GAS6), transcript variant 1, mRNA
product length = 1170

| | |
|---|---|
| Reverse primer | 1 GCCTTTCAGCTACACGAGGT 20 |
| Template | 1467 .TT.A.AG...G........ 1448 |
| Reverse primer | 1 GCCTTTCAGCTACACGAGGT 20 |
| Template | 298 .........G.CTT.....A 317 |

>NM_152609.2 Homo sapiens consortin, connexin sorting protein (CNST), transcript variant 1,
mRNA
product length = 1414

| | |
|---|---|
| Forward primer | 1 TCCTTCGAGTGCTTGTGTTG 20 |
| Template | 2309 CG...A..TG.T........ 2290 |
| Reverse primer | 1 GCCTTTCAGCTACACGAGGT 20 |
| Template | 896 TGT.....CT..T....... 915 |

Ou et al. 2008. Chin Med J (Engl).; 121(17): 1680-7
Primer pair 1

| | Sequence (5′->3′) | Length | Tm | GC % |
|---|---|---|---|---|
| Forward primer | GCTCATCCTAAGCGACGTCT | 20 | 53.90 | 55.00% |
| Reverse primer | GGGGGCATAACTTCTCTTGG | 20 | 52.10 | 55.00% |

>NM_001127493.1 Homo sapiens ankyrin 2, neuronal (ANK2), transcript variant 3, mRNA
product length = 3116

| | |
|---|---|
| Reverse primer | 1 GGGGGCATAACTTCTCTTGG 20 |
| Template | 5004 CA.T........C......A 4985 |
| Reverse primer | 1 GGGGGCATAACTTCTCTTGG 20 |
| Template | 1889 T.T....A...........C 1908 |

product length = 3908

| | |
|---|---|
| Reverse primer | 1 GGGGGCATAACTTCTCTTGG 20 |
| Template | 5004 CA.T........C......A 4985 |
| Reverse primer | 1 GGGGGCATAACTTCTCTTGG 20 |
| Template | 1097 A.T..TGG.......G.... 1116 |

>NM_020977.3 Homo sapiens ankyrin 2, neuronal (ANK2), transcript variant 2, mRNA
product length = 3080

| | |
|---|---|
| Reverse primer | 1 GGGGGCATAACTTCTCTTGG 20 |
| Template | 4910 CA.T........C......A 4891 |
| Reverse primer | 1 GGGGGCATAACTTCTCTTGG 20 |
| Template | 1831 T.T....A...........C 1850 |

product length = 3872

| | |
|---|---|
| Reverse primer | 1 GGGGGCATAACTTCTCTTGG 20 |
| Template | 4910 CA.T........C......A 4891 |
| Reverse primer | 1 GGGGGCATAACTTCTCTTGG 20 |
| Template | 1039 A.T..TGG.......G.... 1058 |

>NM_138289.3 Homo sapiens actin-related protein T1 (ACTRT1), mRNA
product length = 69

| | |
|---|---|
| Reverse primer | 1 GGGGGCATAACTTCTCTTGG 20 |
| Template | 861 T.TA...C..........T. 842 |
| Reverse primer | 1 GGGGGCATAACTTCTCTTGG 20 |
| Template | 793 .C...TT.......C....C 812 |

Matull et al. 2008. Br J Cancer.; 98(10): 1675-81
(Previously published primers used, from Argüeso et al. 2002, see results on the first page of
primer-blast results)
Kraft et al. 2008. Eur Respir J.; 31(1): 43-6
Primer pair 1

| | Sequence (5′->3′) | Length | Tm | GC % |
|---|---|---|---|---|
| Forward primer | AGAGGAGCGGAGAGAGACTCTGT | 23 | 57.81 | 56.52% |
| Reverse primer | CTCCATCTCTCTCTCAGGGTAGTTCT | 26 | 56.23 | 50.00% |

>NM_207360.2 Homo sapiens zinc finger CCCH-type containing 12D (ZC3H12D), mRNA
product length = 64

| | |
|---|---|
| Forward primer | 1 AGAGGAGCGGAGAGAGACTCTGT 23 |
| Template | 3056 T.G.C...A...C.......C.. 3078 |
| Forward primer | 1 AGAGGAGCGGAGAGAGACTCTGT 23 |
| Template | 3119 ....TGA.A...T.......... 3097 |

Wang, K et al. 2007. Chin Med J 120(12): 1051-7
Primer pair 1

| | Sequence (5′->3′) | Length | Tm | GC % |
|---|---|---|---|---|
| Forward primer | TCCGGCCTCATCTTCTCC | 18 | 52.25 | 61.11% |
| Reverse primer | ACTTGGGCACTGGTGCTG | 18 | 54.42 | 61.11% |

>XM_002344536.1 PREDICTED: Homo sapiens similar to mucin (LOC100293983), partial mRNA
product length = 491

| | |
|---|---|
| Forward primer | 1 TCCGGCCTCATCTTCTCC 18 |
| Template | 1546 .................. 1563 |
| Reverse primer | 1 ACTTGGGCACTGGTGCTG 18 |
| Template | 2036 .................. 2019 |

>NM_007030.2 Homo sapiens tubulin polymerization promoting protein (TPPP), mRNA
product length = 104

| | |
|---|---|
| Forward primer | 1 TCCGGCCTCATCTTCTCC 18 |
| Template | 5259 G..CAG.......C.... 5276 |
| Forward primer | 1 TCCGGCCTCATCTTCTCC 18 |
| Template | 5362 ...A...........C.. 5345 |

>XM_001721452.2 PREDICTED: Homo sapiens hypothetical LOC100132779 (LOC100132779),
mRNA
product length = 2249

| | |
|---|---|
| Forward primer | 1 TCCGGCCTCATCTTCTCC 18 |
| Template | 4853 .T.T.G...C........ 4836 |
| Reverse primer | 1 ACTTGGGCACTGGTGCTG 18 |
| Template | 2605 T.......T......A.. 2622 |

Lambiase 2009. Invest Ophthalmol Vis Sci.; 50(10): 4622-30
Primer pair 1

| | Sequence (5′->3′) | Length | Tm | GC % |
|---|---|---|---|---|
| Forward primer | TCCACCATATACCGCCACAGA | 21 | 54.71 | 52.38% |
| Reverse primer | TGGACCGACAGTCACTGTCAAC | 22 | 56.56 | 54.55% |

>XM_002344536.1 PREDICTED: Homo sapiens similar to mucin (LOC100293983), partial mRNA
product length = 103

| | |
|---|---|
| Forward primer | 1 TCCACCATATACCGCCACAGA 21 |
| Template | 805 ..................... 825 |
| Reverse primer | 1 TGGACCGACAGTCACTGTCAAC 22 |
| Template | 907 .....G............... 886 |

Wang, IJ et al. 2009. Mol Vis 15: 108-19
Primer pair 1

| | Sequence (5′->3′) | Length | Tm | GC % |
|---|---|---|---|---|
| Forward primer | GGAAACTGGGCTCTACCCGG | 20 | 56.39 | 65.00% |
| Reverse primer | CATTGTGTGGACGGCGGGGA | 20 | 59.35 | 65.00% |

>NM_022092.2 Homo sapiens CTF18, chromosome transmission fidelity factor 18 homolog (S.
cerevisiae) (CHTF18), mRNA
product length = 1535

| | |
|---|---|
| Forward primer | 1 GGAAACTGGGCTCTACCCGG 20 |
| Template | 1128 ...GG.......GG.....A 1147 |
| Forward primer | 1 GGAAACTGGGCTCTACCCGG 20 |
| Template | 2662 ..GGG...TT.......... 2643 |

>NM_001127714.1 Homo sapiens human immunodeficiency virus type I enhancer binding protein 3
(HIVEP3), transcript variant 2, mRNA
product length = 2702

| | |
|---|---|
| Forward primer | 1 GGAAACTGGGCTCTACCCGG 20 |
| Template | 3235 AC........AA.C....T. 3254 |
| Reverse primer | 1 CATTGTGTGGACGGCGGGGA 20 |
| Template | 5936 GG.........G.CT..... 5917 |

>NM_024503.3 Homo sapiens human immunodeficiency virus type I enhancer binding protein 3
(HIVEP3), mRNA
product length = 2702

| | |
|---|---|
| Forward primer | 1 GGAAACTGGGCTCTACCCGG 20 |
| Template | 3315 AC........AA.C....T. 3334 |
| Reverse primer | 1 CATTGTGTGGACGGCGGGGA 20 |
| Template | 6016 GG.........G.CT..... 5997 |

Yu, DF et al. 2008. Exp Eve Res 86(2): 403-11
Primer pair 1

| | Sequence (5′->3′) | Length | Tm | GC % |
|---|---|---|---|---|
| Forward primer | GACCCAGATCTGCAACACACAC | 22 | 55.89 | 54.55% |
| Reverse primer | TGGATGATCAGGCTCCTATGGT | 22 | 54.34 | 50.00% |

>XM_002344536.1 PREDICTED: Homo sapiens similar to mucin (LOC100293983), partial mRNA
product length = 360

| | |
|---|---|
| Forward primer | 1 GACCCAGATCTGCAACACACAC 22 |
| Template | 2496 ...................... 2517 |
| Reverse primer | 1 TGGATGATCAGGCTCCTATGGT 22 |
| Template | 2855 ...................... 2834 |

>NM_003626.2 Homo sapiens protein tyrosine phosphatase, receptor type, f polypeptide (PTPRF),
interacting protein (liprin), alpha 1 (PPFIA1), transcript variant 2, mRNA
product length = 3609

| | |
|---|---|
| Forward primer | 1 GACCCAGATCTGCAACACACAC 22 |
| Template | 1321 ..G.A.A.G........G.... 1342 |
| Forward primer | 1 GACCCAGATCTGCAACACACAC 22 |
| Template | 4929 A.G...C.........TT.... 4908 |

Based on the last reference sequence of MUC5AC available at the time of these analyses, it seems evident that the designs presented above are not suitable. The assay that obtains the closest hit to the target sequence is a MUC5AC predicted sequence, which is discarded nowadays due to its high divergence from the current validated sequence (NM_{—}017511, SEQ ID NO:1). The rest of the hits are predicted genes, the MUC5B gene, predicted genes similar to mucin genes, partial molecular clones or other sequences with no relation to the target of interest.

1.1.2 Design of New Oligonucleotide Primers

It is described here the workflow carried out for the final design of the RT-PCR for the diagnosis of limbal deficiency, based on the detection of the MUC5AC transcript in corneal epithelium. This workflow justifies the region of the transcript selected in the last design step, based on different assays and bioinformatic analyses.

Although the PCR has multiple advantages, its main disadvantage relies on that critical conditions must be optimized for each design: annealing temperature, primer concentration, polymerase concentration, number of amplification cycles, additives, etc.

Primarily, the most updated and validated (SEQ ID NO: 1) sequence of the transcript of interest was downloaded from public databases, and two primer pairs were selected in the same region of the transcript. In this first approach, the inventors looked for local regions, adequate for PCR design a priori, putting all the effort in the selection of an adequate fragment based on its nucleotidic composition and repetitive elements.

However, in this first experimental approach, it was not possible to obtain specific amplification products, so a longer region was used for the primer design, increasing the number of primer pairs to test. All these new primer pairs were tested and tuned individually, with no good results. Two of the primers tested were: MUC5AC-Fw-TGGTCCTCAGAGATGGGAAG (SEQ ID NO:19) and MUC5AC-Rv-GAAACCCCAATAGCCCATTC (SEQ ID NO:20), amplifying a region in the cDNA of MUC5AC between the nucleotides 18429 and 18691.

At this point, it was supposed that there were intrinsic problems for the absence of specific amplification in all of the assays, such as structural or steric problems.

Due to the nature of the genetic code (only 4 alternative nucleotides and the requirement of a reverse complementary strand for the stabilization in double stranded structures), regions intrinsically difficult to amplify are found throughout the genome/transcriptome. MUC5AC transcript is one of such regions due to the reasons described below:
Highly repetitive regions (both of the same nucleotide and of different combinations of different nucleotides).
High G/C content, raising the energetic requirements for melting double stranded structures.
Genes/transcripts originated through successive segmental duplications of one or several initial elements.
Regions with reverse complementary fragments repeated in tandem (a fragment after which we can identify the reverse complementary of the first fragment). This regions are relatively common in RNA (RNA is single stranded, while double strands possess a higher stability), but they are extremely abundant throughout the sequence of MUC5AC transcript analyzed in small fragments.

All these features make the MUC5AC transcript intrinsically difficult to specifically amplify. Moreover, another problematic issue in the PCR design is the specificity, to identify and experimentally obtain a fragment that is only found once throughout the genome/transcriptome and that does neither amplify variants (with insertions, deletions, etc) nor pseudogenes or non-coding regions. In this regard, the specificity of MUC5AC transcript is highly compromised, with high degrees of homology between different members of the family of genes within the MUC family. This specific feature points to a common ancestor gene, which rendered different genes through duplications and subsequent divergence (changes in sequence). This homology is nearly complete for MUC5AC and MUC5B. This is because, besides the successive duplications, both RNAs are transcribed from the same locus (gene), they are alternatively spliced RNAs transcribed from the same genomic region. This feature is generating the specificity problems.

Using bioinformatic methods to visualize the RNA structure and homology of sequences (Nucleic Acid Dot Plots, http://www.vivo.colostate.edu/molkit/dnadot/), the inventors showed that the high homology and structuration was not only found between different members of the family of mucins, but was also found throughout the sequence of the MUC5AC transcript. These findings suggest that MUC5AC gene was originated through successive segmental duplications of an ancestral fragment, which makes the design of a specific PCR even more difficult.

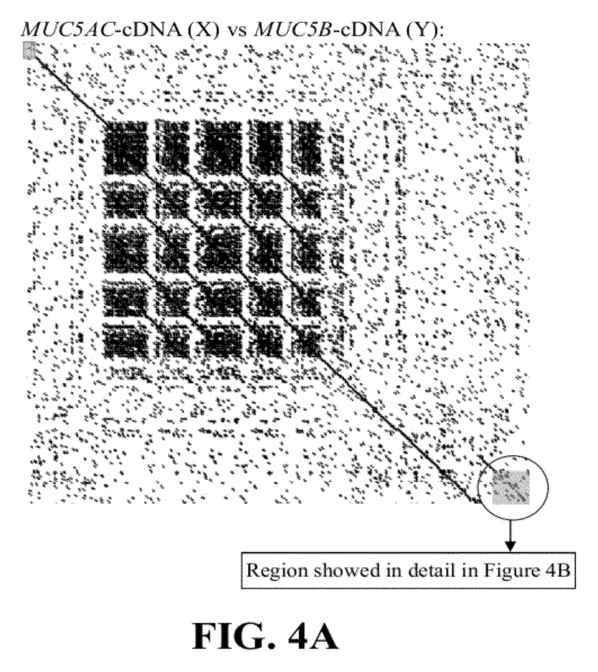

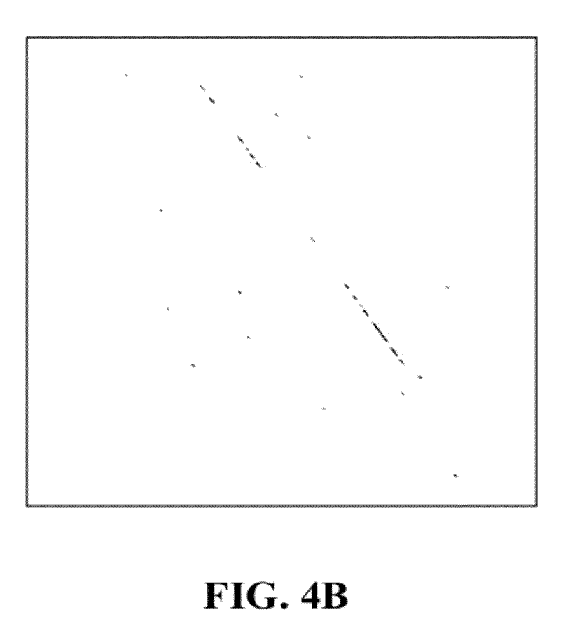

Nevertheless, while these analyses were performed, 2 short regions of about 420 bp (1-420) and 1311 (17440-18750) (top left and lower right corner in FIG. 4A, respectively) were identified, that were divergent between MUC5AC and MUC5B transcript, and still poorly structurated for an optimized primer design. The methodology for the selection of this short fragment is shown below.

Traditional sequence manipulation and analysis tools are not valid for the identification of differences, homologies and repetitive regions in a global scale. This is because the transcript of interest is very large (MUC5AC: 17.7 Kb) and traditional tools only allow for basic alignments (large duplications, for example, are impossible to detect, as any of the duplications of one of the sequences may align with any of the duplications of the other sequence).

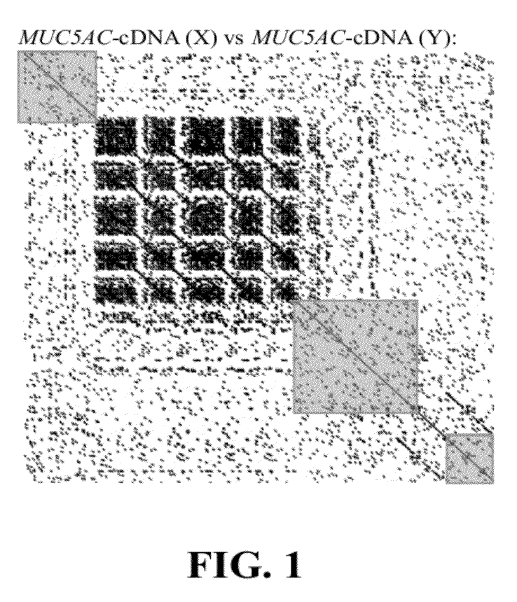

FIG. 1 shows a plot in which two sequences are compared, in X and Y axis, showing the result as a dot plot. Dots represent sequence homology and white spaces represent divergence. X axis represents the total length of the sequence input as DNA1, while Y axis represents the total length of the sequence input as DNA2. The program allows clicking on the desired dots with the mouse, showing their position in both sequences. Therefore, when comparing a sequence with itself, at least one diagonal continuous line should be obtained, representing complete homology in the same positions throughout the sequence. In FIG. 1, the diagonal continuous line can be clearly seen, showing complete homology throughout the whole sequence of both transcripts; this is because they are actually the same sequence. Nevertheless, it is also possible to see multiple dots far away from the diagonal, representing homology between different parts of the same sequence. Given that only 4 alternative nucleotides exist, it is common to find some degree of structuration when comparing a sequence to itself. But in this case several extra diagonals can be seen, far away from the main one, showing continuous and extensive homology of fragments of the transcript throughout the sequence, in different shifted positions (repetitive elements that are found many times throughout the sequence, mainly in the first half of the sequence). As a direct consequence, only the regions in the rectangle should be considered for the primer design.

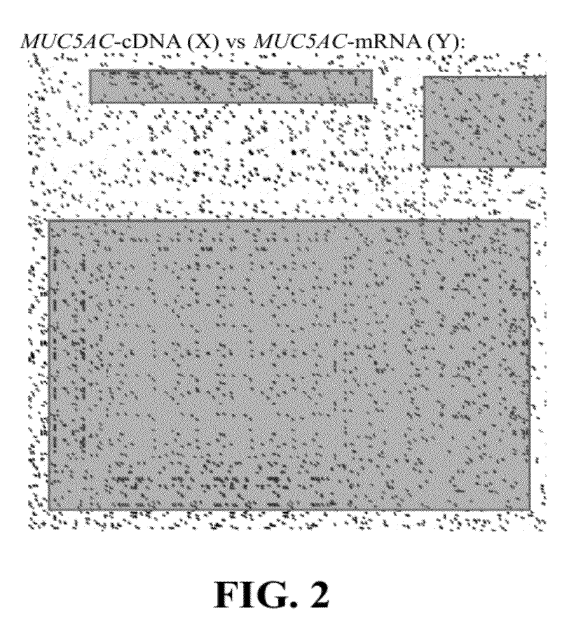

On the other hand, since the aim is to characterize palindromic fragments, the sequence of interest (MUC5AC-cDNA) should be compared with its reverse complementary (MUC5AC-mRNA, the original transcript) (FIG. 2). Through the same deductive process as for FIG. 1, this Plot suggests that, due to the structuration of palindromic sequences, primers should not be designed in the regions within the rectangles, because of their reverse complementary homology that produces double stranded regions in a single stranded molecule (see the distribution of dots with some structuration, difficult to appreciate at a glance). Although it seems that little homology is found at a glance, this type of structuration is of paramount importance for PCR, due to the marked tendency of palindromic sequences to anneal with each other. However, this analysis is global, with not much specific information; it is a draft approach that will permit to globally select a specific region to perform the primer design. After selecting the proper region, the RNAfold application described above will be used as a more adequate tool to characterize palindromic sequences and secondary structures generated as a consequence. This analysis will be performed over the final fragments selected to amplify.

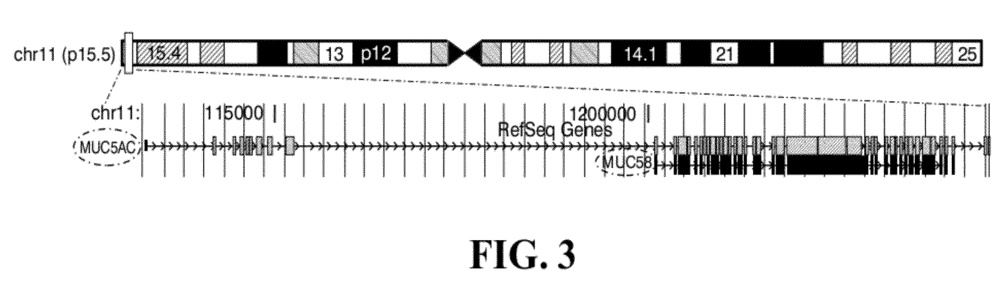

On the other hand, it must be taken into account during primer design that MUC5AC is a member of a wide family of glycoproteins called mucins (MUC). In this case, most of the family members arose from a common ancestor, so high levels of sequence homology are found between them. Although many forms of mucins exist, only MUC5B was found to show nearly complete homology with MUC5AC. When a characterization of both genes in their genomic context was performed, it was found that both transcripts are product of alternative transcription initiation and splicing of the same locus (FIG. 3).

Therefore, the design of a PCR that specifically amplifies MUC5AC is crucial; otherwise, MUC5B expression could also be detected, with the consequent risk of false positives. In this regard, it must be taken into account that, given the high homology, amplification products of the same length could be probably obtained. This would make the amplified products obtained by both sequences very difficult to discriminate, even using hybridization probes, electrophoresis or sequencing.

FIG. 4 shows a comparison of cDNA sequences of MUC5AC and MUC5B transcripts by means of Nucleic Acid Dot Plots. A high level of homology throughout the sequences can be seen, although a shift in the positions between the two sequences is observed. Moreover, the same repetitive blocks found in the comparison of MUC5AC cDNA with itself can still be seen in this case. Following the same interpretation performed during all the comparisons, primers should only be designed in the short regions in the rectangles (upper left and lower right corners), as it seems to be the only region with a relatively low homology. After all the analyses performed by the inventors, the target regions were reduced to nucleotides between 1 and 420 (421 bp) and between 17440 and 18750 (1311 bp) (cDNA positions).

Browsing over the original plot, the exact position of a dot in the plot and the exact length of the fragment in rectangle can be identified. The comparison of the 4 different plots presented (FIGS. 1, 2, 4A and 4B) here shows that all of them have promising results for the fragments in the rectangles (upper left and bottom right corners) in FIG. 4A (MUC5AC vs MUC5B). To calculate the position of these fragments, MUC5AC-cDNA coordinates must be used as reference in all cases (in X axis for all the plots shown).

Once the specificity of the selected fragments was demonstrated (unique sequence throughout the genome or transcriptome, by means of BLAST/BLAT), the primer design using as a template the regions mentioned above was started and is explained in the following section.

1.1.3 Design of the Primers in the Candidate Regions

Once the highly specific short fragments with low levels of secondary structuration were determined, a massive primer design (5 forward primers and 8 reverse primers selected for conventional RT-PCR) was performed. The primers sequences and the fragments expected to be amplified within the sequence of the MUC5AC cDNA is shown in the following table:

| | | | |
|---|---|---|---|
| MUC5AC- | CTGAGCTGCTTGGTGGTTTCAGGC | MUC5AC-R1 | 128-337 |
| F1 | SEQ ID NO: 4 | SEQ ID | |
| | | NO: 9 | |
| | | MUC5AC-R2 | 128-343 |
| | | SEQ ID | |
| | | NO: 10 | |
| | | MUC5AC-R3 | 128-379 |
| | | SEQ ID | |
| | | NO: 11 | |
| | | MUC5AC-R4 | 128-384 |
| | | SEQ ID | |
| | | NO: 12 | |
| | | MUC5AC-R5 | 128-385 |
| | | SEQ ID | |
| | | NO: 13 | |
| | | MUC5AC-R6 | 128-387 |
| | | SEQ ID | |
| | | NO: 14 | |
| | | MUC5AC-R7 | 128-388 |
| | | SEQ ID | |
| | | NO: 15 | |
| | | MUC5AC-R8 | 128-236 |
| | | SEQ ID | |
| | | NO: 16 | |
| MUC5AC- | GAGCTGCTTGGTGGTTTCAGGCTG | MUC5AC-R1 | 130-337 |
| F2 | SEQ ID NO: 5 | MUC5AC-R2 | 130-343 |
| | | MUC5AC-R3 | 130-379 |
| | | MUC5AC-R4 | 130-384 |
| | | MUC5AC-R5 | 130-385 |
| | | MUC5AC-R6 | 130-387 |
| | | MUC5AC-R7 | 130-388 |
| | | MUC5AC-R8 | 130-236 |
| MUC5AC- | CTGCTTGGTGGTTTCAGGCTGAATG | MUC5AC-R1 | 133-337 |
| F3 | SEQ ID NO: 6 | MUC5AC-R2 | 133-343 |
| | | MUC5AC-R3 | 133-379 |
| | | MUC5AC-R4 | 133-384 |
| | | MUC5AC-R5 | 133-385 |
| | | MUC5AC-R6 | 133-387 |
| | | MUC5AC-R7 | 133-388 |
| | | MUC5AC-R8 | 133-236 |
| MUC5AC- | TTCCAAGGGCACAGGGCAGAGGCAG | MUC5AC-R1 | 164-337 |
| F4 | SEQ ID NO: 7 | MUC5AC-R2 | 164-343 |
| | | MUC5AC-R3 | 164-379 |
| | | MUC5AC-R4 | 164-384 |
| | | MUC5AC-R5 | 164-385 |
| | | MUC5AC-R6 | 164-387 |
| | | MUC5AC-R7 | 164-388 |
| | | MUC5AC-R8 | 164-236 |
| MUC5AC- | GCTGCTTGGTGGTTTCAGGCTGAAT | MUC5AC-R1 | 132-337 |
| F5 | SEQ ID NO: 8 | MUC5AC-R2 | 132-343 |
| | | MUC5AC-R3 | 132-379 |
| | | MUC5AC-R4 | 132-384 |
| | | MUC5AC-R5 | 132-385 |
| | | MUC5AC-R6 | 132-387 |
| | | MUC5AC-R7 | 132-388 |
| | | MUC5AC-R8 | 132-236 |

All the primers were centered on the same region (a priori, the best sub-region for primer design in the selected 420 bp fragment in the top left corner of FIG. 4A), with some shift in the positions of the different primers. This allowed testing all the possible combinations between forward and reverse primers. Nevertheless, only one specific amplification product was obtained for one of the combination of primers (F2-R3) corresponding to the amplification of the fragment 130-379 of the MUC5AC transcript. However, the inventors were not able to obtain specific amplification for any of the combinations between different forward and reverse primers.

The extensive problems shown for the specific amplification of MUC5AC transcript encouraged the inventors to perform two further designs in combination with a fluorescence probe (TaqMan like), which would allow the Real-Time detection of the amplification reaction and partially limit the unspecificity problems. Adding a Real-Time detection system accelerates the diagnostic procedure and reduces the risk of cross contamination with PCR product. Moreover, it allows adding the diagnosis method a quantitative value after establishing the detection limit (minimum amount of starting template) and dynamic range (in which the relative quantity can be reliably calculated). The primers tested in this final step were:

(SEQ ID NO: 17)
MUC5AC-RT-F1 CTCTGCACCCACACCACA
(SEQ ID NO: 18)
MUC5AC-RT-R1 AGGAAGCCTCTGGGAAGG
(SEQ ID NO: 2)
MUC5AC-RT-F2 CCTGCAAGCCTCCAGGTAG
(SEQ ID NO: 3)
MUC5AC-RT-R2 CTGCTCCACTGGCTTTGG

At this time the inventors designed one extra primer pair (MUC5AC-RT-F1-MUC5AC-RT-R1) in the region 1 to 420, whereas another pair (MUC5AC-RT-F2-MUC5AC-RT-R2) was designed in the region from nucleotides 17440 to 18750, which also showed interesting results during the analyses.

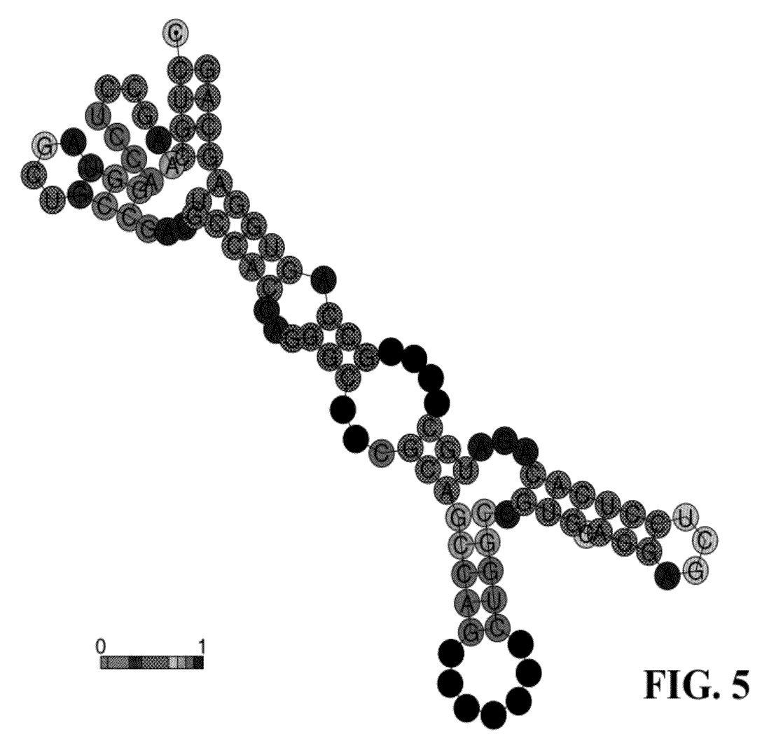

Simultaneously, the amplicons produced by these last designs were tested for RNA structuration, through RNAfold Web Server. The results are shown in FIG. 5 and indicate that, even the most suitable regions to design the PCR in the transcript (shaded in green in FIG. 4A) show high degrees of secondary structuration due to palindromic sequences. However, in the case of the final selected fragment (the last amplicon of choice, constituting the diagnosis method), the region amplified with the primers MUC5AC-RT-F2 and MUC5AC-RT-R2, a crucial difference is found. The colour intensity of the graphical representation of the possible secondary structures changes with the probability of the annealings: a value of 1 indicates that is more probable that annealings will happen and a value of 0 indicates that the annealing is not probable. In contrast to previous analyses, a considerable structuration can be seen but, in general, with low probabilities of happening. Nucleotides shaded with colours of higher probabilities (closer to 1) are those that do not anneal according to our analyses. This means that the annealing between palindromic sequences is in general weaker or less probable, whereas the non-annealing regions (desirable for a successful PCR) are much more probable. The analysis for the last selected fragment (comprised between primers MUC5AC-RT-F2 and MUC5AC-RT-R2) is shown in FIG. 5.

Finally, it was possible to obtain specific fragments with one of the designs in combination with the fluorescence probe (5′-CTGGCTGC-3′ labeled with 6-FAM at the 5′ end). Subsequent assays produced good quality real-time results, although some degree of inter-individually variable unspecificity in agarose gels was found, which was reduced considerably optimizing the next assays.

The definitive proof of the suitability, validity and specificity of the amplified fragment was the sequencing of the specific band with the expected length, cutting off from the agarose gel and purifying with a gel extraction kit. The data obtained by sequencing was queried against GenBank/Genomes databases of NCBI (by means of its search algorithm BLAST: http://blast.ncbi.nlm.nih.gov/Blast.cgi) or UCSC Genome Browser database, by means of its search algorithm BLAT (http://genome.ucsc.edu/cgi-bin/hgBlat?command=start) (see below the results from BLAT).

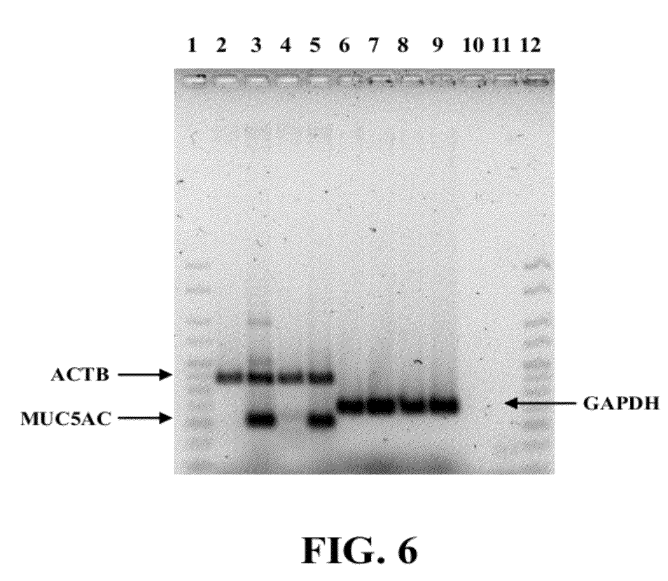

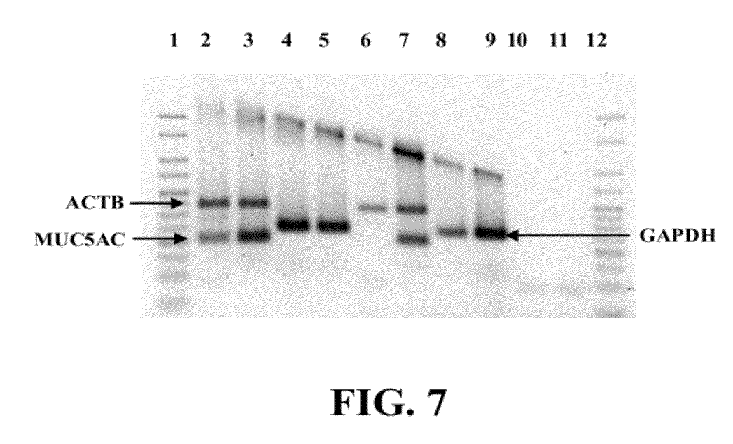

The final oligonucleotide primers [MUC5AC-RT-F2 (SEQ ID NO: 2) and MUC5AC-RT-R2 (SEQ ID NO: 3)] were tested with some patients. The results obtained for different patients in cornea and conjunctiva are shown in FIGS. 6 and 7, amplifying MUC5AC and two housekeeping genes (ACTB and GAPDH, used as quality controls for the starting template).

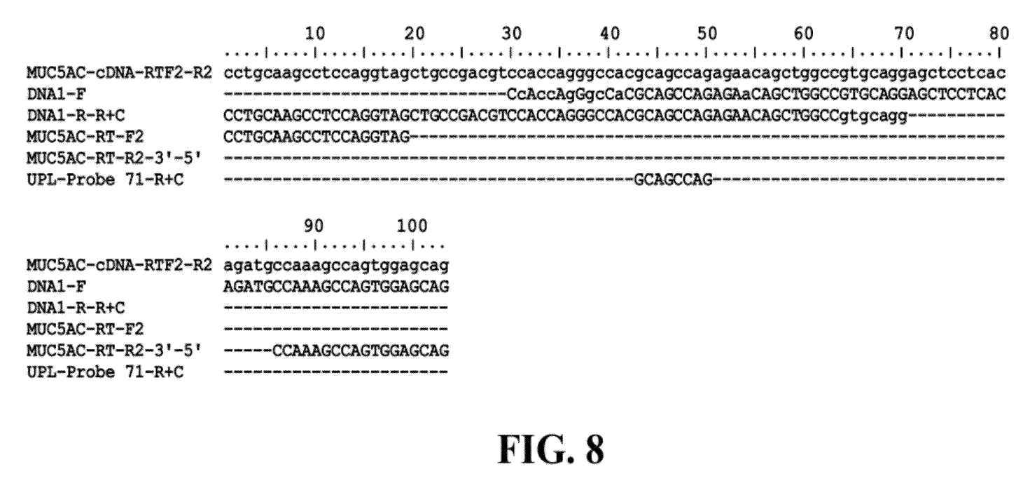

FIG. 8 shows the alignment of both oligonucleotide primers with the reference sequence (SEQ ID NO: 1) (first row) sequenced with the primers, including the forward primer, the reverse complement of the reverse primer and the reverse complement of the hybridization probe. It must be noted that for all the steps in the analyses and for the primer design, the reverse complementary sequence of MUC5AC RNA was used, given that PCR will be performed using cDNA (reverse complementary of the RNA) as the starting template to amplify. Primers are located exactly flanking this fragment, whereas the probe is located in an internal position (FIG. 8). When amplification is performed, a 5′-3′ exonuclease activity enzyme will degrade the probe, separating the quencher and the fluorescent molecule and producing a signal that is detectable above a threshold.

Two specific hits were obtained during the BLAT search, the specific fragment of 103 bp and another unspecific fragment. However, the only unspecificity that produces the target sequence is comprised within the first 26 base pairs, but spanning 425 base pairs in chromosome 9. This incomplete hit does not imply a problem, because it does not include both flanks of the sequence, so that one of the primers (the downstream, in this case, the forward primer for the cDNA) will never anneal. As a consequence, the amplification reaction will have a linear dynamic instead of exponential, so the amplification will never reach a sufficient number of copies to be detected by electrophoretic methods.

On the other hand, the probe is located far away from the sequence that produces this partial hit, so the Real-Time PCR will never produce a signal for this fragment and will not be detected.

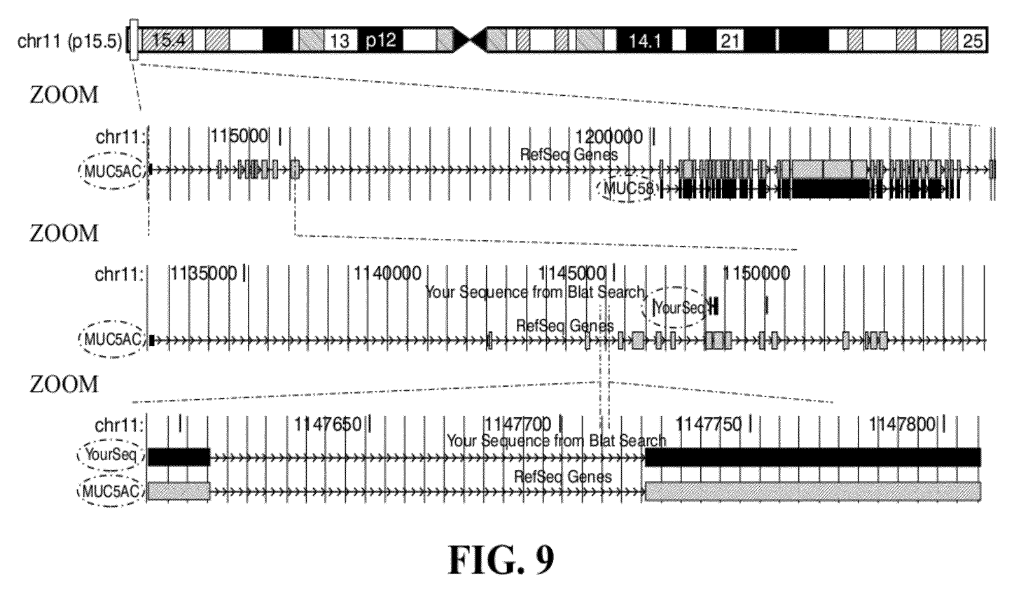

Location of the MUC5AC gene and of the target fragment of interest in the human genome is shown in FIG. 9. Though the amplification is performed over RNA, the exact locations and coordinates must be referenced to the positions of the genome. The target sequence corresponds to the solid lines seen in “YourSeq”, whereas the narrow lines with the arrows pointing the sense of the transcription correspond to introns. Introns are never present in RNA due to the splicing process, and this feature makes possible to specifically amplify RNA/cDNA, and not genomic DNA, with an appropriate design. Once again, this diagram shows the high homology between MUC5AC and MUC5B, demonstrating that both RNAs are transcribed from the same locus, by means of alternative splicing and transcription beginning. Nevertheless, the selected region (YourSeq) is located in a region not shared with MUC5B.

### 1.2 Samples Used

Samples were collected from patients selected from outpatient services of some Spanish hospitals, namely, Hospital de Cruces (Bilbao), Hospital Clinico (Madrid), Hospital La Paz (Madrid), Hospital 12 de Octubre (Madrid) and Hospital Donostia (San Sebastian). The inclusion criteria were established by means of anamnesis and clinical assessment and included patients with suspected LSCD. A patient was considered to present signs of LSCD when in the slit lamp examination the patient presented a corneal vascularization as well as signs of corneal erosions and permanent instability of the tear film.

A cornea sample and a conjunctiva sample were taken from the selected patients by means of impression cytology for their analysis by means of RT-PCR. Furthermore, a cornea sample and another conjunctiva sample from the same eye were also taken from them by impression cytology, to analyze them by means of PAS-hematoxylin staining. Simultaneously, a cornea sample from a subject without LSCD was taken by means of impression cytology. The effectiveness of both methods (PAS-hematoxylin staining and RT-PCR) can thus be evaluated and the reference samples can be obtained to compare the values at the time of the quantification.

For the collection of samples, circular membranes with a size considerably smaller than the diameter of the cornea for the extraction of RNA, and rectangular membranes with a peak at their end (5×5 mm) for a correct orientation for the PAS-hematoxylin staining were used. In both cases the same paper was used, Millipore nitrocellulose paper (HAWP304).

The impression cytology samples intended for the extraction of RNA were stored in microtubes containing “RNAprotect cell reagent” (QIAgen P/N: 76526), for the purpose of protecting the RNA and so that the samples could be preserved at room temperature until their analysis, preventing their degradation. For the PAS-hematoxylin staining, the impression cytology samples were fixed with 96% ethanol and subsequently stained with PAS-hematoxylin according to the protocol followed by Locquin and Langeron and modified by Rivas et al. (Rivas L et al. Acta Ophthalmol 1991; 69: 371-376). The evaluation was carried out by means of optical microscopy.

### 1.3 Extraction and Quantification of RNA

The RNeasy PLUS Micro kit (Qiagen, P/N: 74034) was used for the extraction of RNA, and all the steps were carried out at room temperature (15° C.-25° C.). Briefly, the membrane containing the sample was transferred to a new RNAse-free tube and centrifuged for 10 seconds at 3,500 rpm to eliminate the RNA stabilization solution. The membrane was again transferred to another tube and 350 μl of the Buffer RLT Plus [Qiagen] were added. The cells were lysed vortexing for 2 minutes. The lysate and the membrane were transferred to a “gDNA Eliminator” column [Qiagen] and centrifuged for 30 seconds at equal to or higher than 8,000 g (equal to or higher than 10,000 rpm). A volume of 70% ethanol (350 μl) was added to the resulting flow-through and mixed by pippeting. The entire volume (including any precipitate formed) was transferred to an “RNeasy MinElute” column [Qiagen] with its collection tube and centrifuged for 15 seconds at equal to or higher than 8,000 g (equal to or higher than 10,000 rpm). The flow-through and the collection tube were discarded and 700 μl of Buffer RW1 [Qiagen] were added to the column and centrifuged for 15 seconds at equal to or higher than 8,000 g (equal to or higher than 10,000 rpm). Subsequently, 500 μl of buffer RPE [Qiagen] were added to the column and it was centrifuged again. The column was washed with 500 μl of 80% ethanol. Finally, (depending of the starting RNA concentration). RNA was eluted by centrifugation for 1 minute at maximum speed. The eluted RNA was placed again in the center of the column, reincubated for 10 minutes at room temperature, and eluted again. The RNA obtained can be preserved at −80° C. for 1 year.

### 1.4 Retrotranscription (RT) Reaction

For the retrotranscription of RNA, the “Transcriptor First Strand cDNA Synthesis” kit, from Roche (P/N: 04 379 012 001), with a combination of Oligo(dT) 18 and Random Hexamers as primers of the reaction, was used.

Table 1 indicates the composition of the reaction mixture for the standard retrotranscription of MUC5AC. The volume of RNA can vary, depending on the concentration obtained in the extraction.

TABLE 1
Reaction mixture for the RT

| | |
|---|---|
| MIX | 1.0 |
| Premix (denat) | |
| 5.0 H₂0 (v7/9) | 5.0 |
| 1.0 Oligo dT | 1.0 |
| 2.0 Rand Hex (v6) | 2.0 |
| 5.0 RNA | 5.0 |
| | 13.0 |

65° C. 10 min 4° C.
MIX (RT)

| | |
|---|---|
| 4.0 Buffer 5× (v2) | 4.0 |
| 0.5 RNAse inh. (v3) | 0.5 |
| 2.0 dNTPs (v4) | 2.0 |
| 0.5 RTase (v1) | 0.5 |
| | 7.0 |
| Total | 20.0 |

The first step of the reaction (65° C., 10 minutes, the 4° C.) consisted of an initial denaturation before adding the rest of the reagents (of the second part of the mixture). Once the 10 minutes have elapsed, the rest of the reagents were added and the retrotranscription was carried out. The retrotranscription cycles were: 10 minutes at 25° C., 30 minutes at 55° C., 5 minutes at 85° C. and finally it was maintained at 4° C.

The cDNA obtained at the end of the protocol is used directly, without prior purification as a template for the PCR.

### 1.5 Real-Time PCR (RT-PCR)

1-10 μl (depending of the starting RNA concentration) of non-purified retrotranscription product (cDNA) were used as a template for the RT-PCR. To that end, the following oligonucleotide primers were used:
SEQ ID NO: 2 (MUC5AC-RT-F2) and
SEQ ID NO: 3 (MUC5AC-RT-R2)
   and the Probe #71 from Roche, specific for the amplification product, which has the nucleotide sequence 5′-CTGGCTGC-3′ and is labeled at the 5′ end with the fluorophore 6-FAM, compatible for the detection thereof in real-time PCR equipment.

The reaction mixture was prepared with a final volume of 20 μl with the reagents and volumes indicated in Table 2.

TABLE 2
Reaction mixture of the qPCR

| MIX | 1 | | |
|---|---|---|---|
| H₂O | 10.76 | | |
| Buffer 10× | 2 | | |
| MgCl₂ 50 mM | 1.4 | | |
| dNTPs 25 mM | 0.64 | | |
| F primer | 1.2 | | |
| R primer | 1.2 | | |
| Probe #71 | 0.6 | | |
| Taq | 1.2 | | |
| | 18 | μl | MIX + 2 μl cDNA |

F primer: SEQ ID NO: 2 (MUC5AC-RT-F2); R primer: SEQ ID NO: 3 (MUC5AC-RT-R2); Probe #71 (Roche): 5′-CTGGCTGC-3′ labeled at the 5′ end with 6-FAM.

The relative quantification of the amplifications was analyzed using the “iQ5 optical system software” program included in the iQ5 Real time-PCR equipment (Biorad). The conditions of the PCR are indicated in Table 3.

TABLE 3
PCR conditions

| | | |
|---|---|---|
| Step 1 | Step 2 | Step 3 |

| | X40 | | | | |
|---|---|---|---|---|---|
| 96° C. | 96° C. | 60° C. | 72° C. | 72° C. | 15° C. |
| | | | (RT) | | |
| 3 min | 20 s | 20 s | 10 s | 5 min | ∞ |

### Results of the First Group of Patients

A prospective study has been conducted on 14 eyes of 13 patients (7 men and 6 women) with limbal stem cell deficiency caused by ocular burns (2 eyes), Stevens-Johnson syndrome (2 eyes), ocular cicatricial pemphigoid (2 eyes), contact lens wearers (1 eye), others (7). The study inclusion criteria were established by means of anamnesis and clinical assessment. A patient was considered to present signs of limbal stem cell deficiency when in the slit lamp examination the patient presented a corneal vascularization as well as signs of corneal erosions and permanent instability of the tear film.

The analyzed cornea and conjunctiva samples were collected by means of impression cytology and processes as has been indicated in the previous sections.

The transcript of the mucin gene (MUC5AC) was detected in 10 of the 14 corneal epithelium samples analyzed by RT-PCR. However, it was only possible to diagnose LSCD by means of conventional impression cytology with PAS-hematoxylin in 5 patients analyzed.

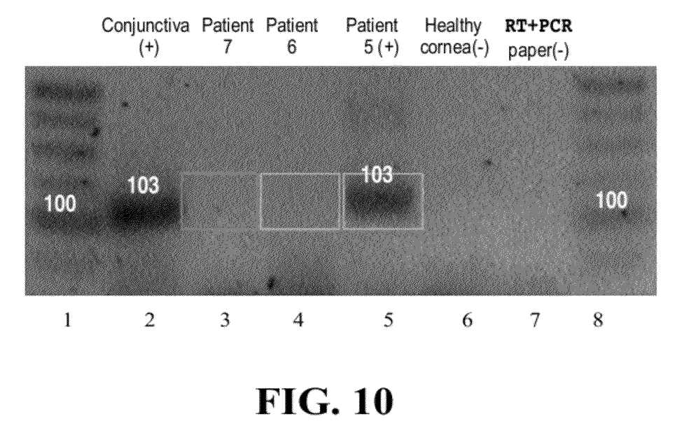

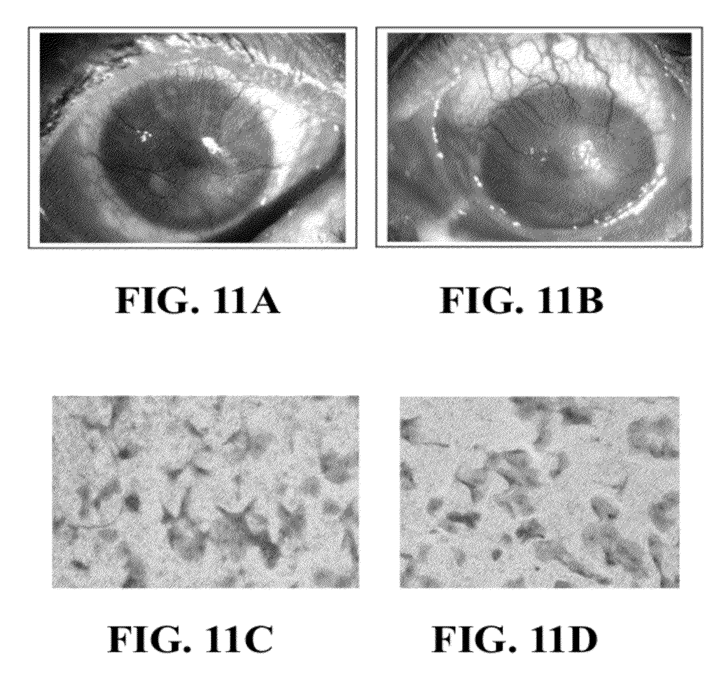

The results obtained are shown in FIGS. 10-19 (corresponding to Cases 1-13 mentioned below).

### Cases 1, 2 and 3: Samples from Patients 5, 6 and 7

FIG. 10 shows the results of the analysis of patients 5, 6 and 7 in one and the same gel by means of detecting the MUC5AC transcript. In these cases, the results corresponding to the impression cytology and PAS-hematoxylin staining are not available. As can be seen in said FIG. 10, LSCD is confirmed for patient 5 but not for patients 6 and 7 by means of amplifying the specific 103 bp fragment of the mRNA of the MUC5AC gene.

The results obtained in the patients analyzed by comparing the diagnoses of the impression cytology and PAS-hematoxylin staining with those obtained by means of amplifying a specific 103 bp fragment of the mRNA of the MUC5AC gene by RT-PCR are shown below.

### Case 4: Sample from Patient 0216

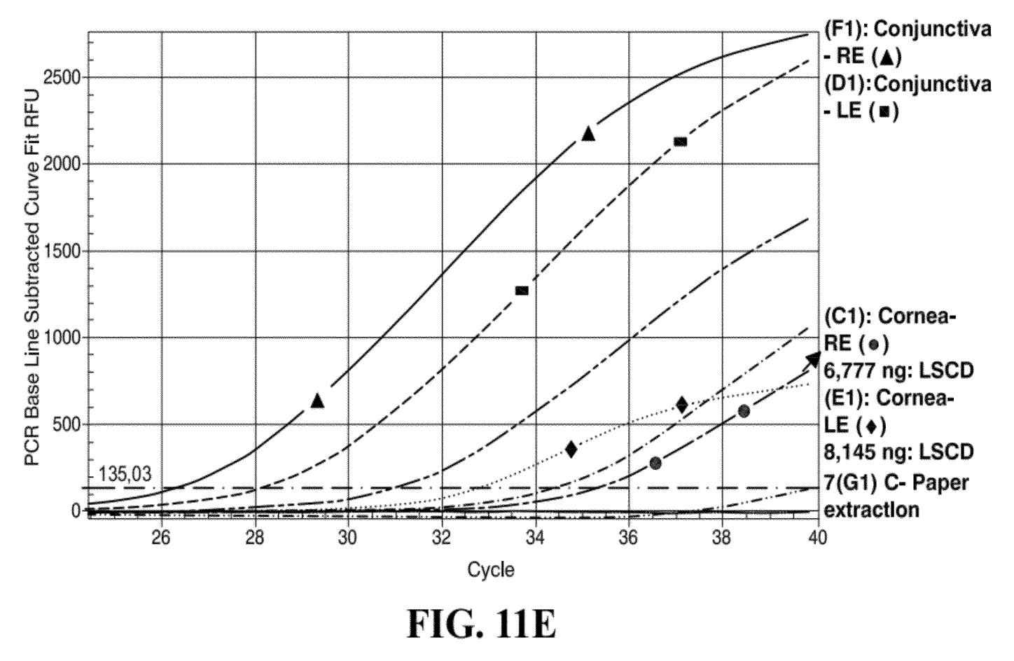

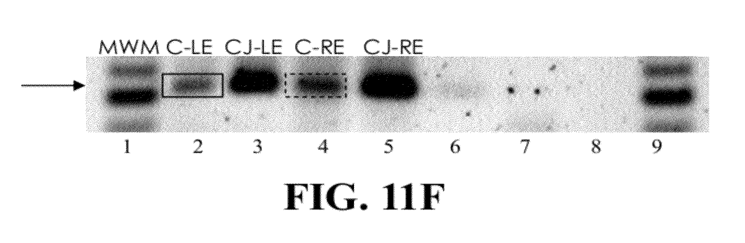

Patient with LSCD, diagnosed by slit lamp (FIGS. 11A and 11B), by impression cytology and PAS-hematoxylin staining (FIGS. 11C and 11D) and by means of RT-qPCR (FIGS. 11E and 11F). The presence of goblet cells in the cornea is observed (FIGS. 11C and 11D). FIG. 11E shows amplification curves that do not cross the threshold (no amplification, negative samples) and amplification curves that do cross the threshold at some point (amplification, positive samples). The curve (C1) corresponds to the cornea sample from the left eye of lane 2 in the agarose gel (FIG. 11F) and the curve (E1) corresponds to the cornea sample from the right eye of lane 4 in the agarose gel (FIG. 11F). As can be seen in said FIG. 11F, the conjunctiva samples give very high values, indicating a larger amount of mRNA, and correspond to the curves (D1) (conjunctiva of the left eye) and (F1) (conjunctiva of the right eye) [lanes 3 and 5, respectively, in the agarose gel (FIG. 11F)]. Likewise, the LSCD in both eyes is confirmed by means of RT-PCR due to the amplification of a specific 103 bp fragment of the mRNA of the MUC5AC gene (FIG. 11F).

### Case 5: Sample from Patient 076

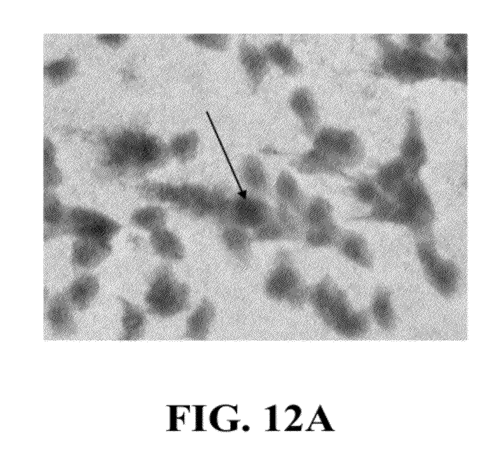

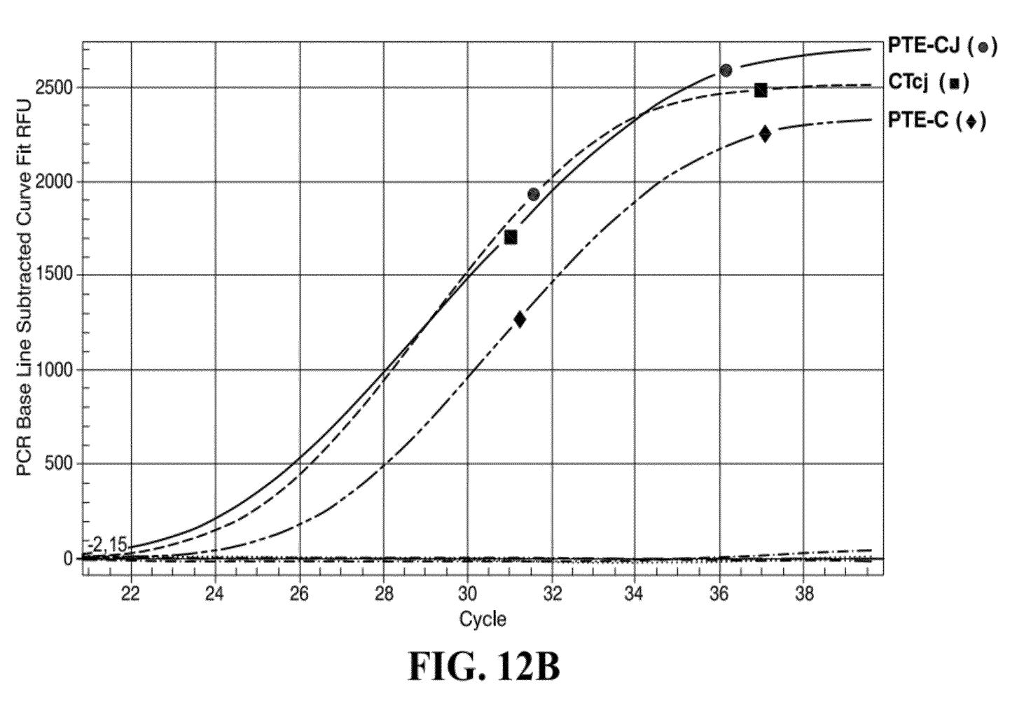

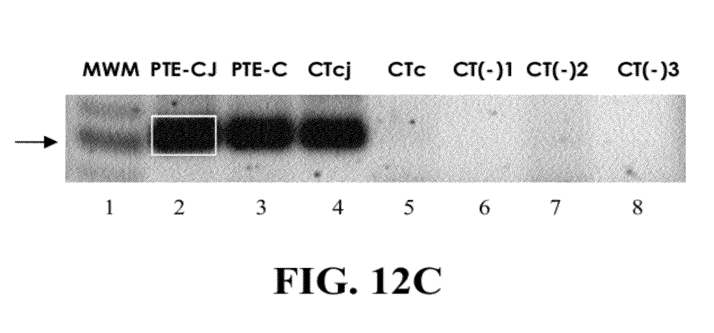

Patient clinically diagnosed with LSCD. The impression cytology and PAS-hematoxylin staining confirm the presence of goblet cells in the cornea (FIG. 12A). FIG. 12B shows amplification curves that do not cross the threshold (no amplification, negative samples) and amplification curves that do cross the threshold at some point (amplification, positive samples). Likewise, the LSCD is confirmed by means of RT-PCR due to the amplification of the specific 103 bp fragment of the mRNA of the MUC5AC gene (FIG. 12C).

### Case 6: Sample from Patient 0121

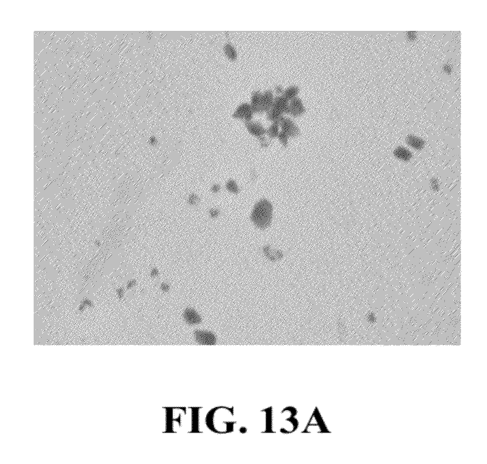

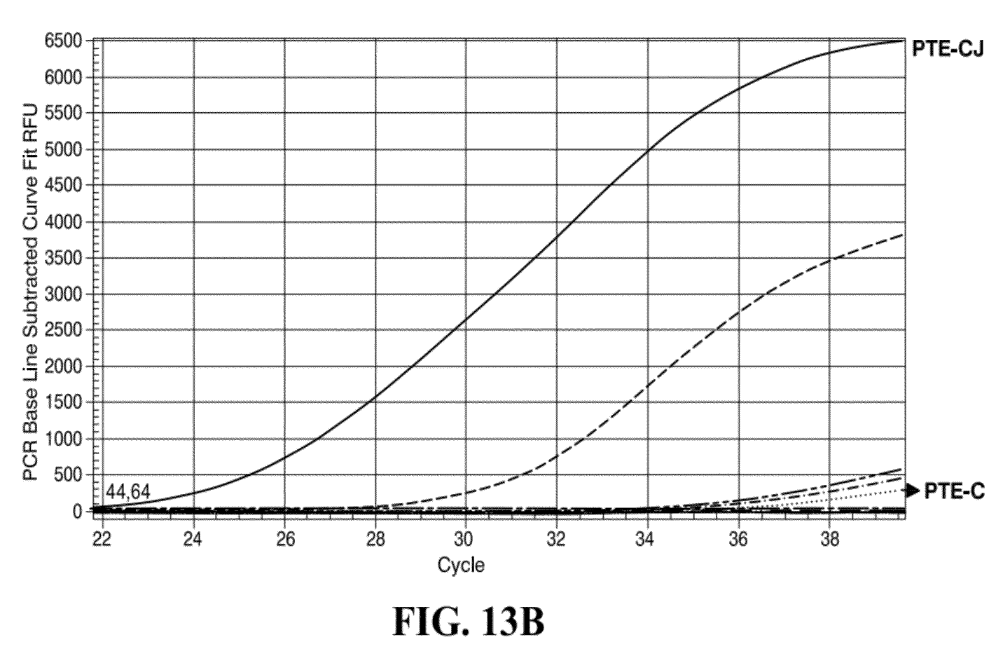

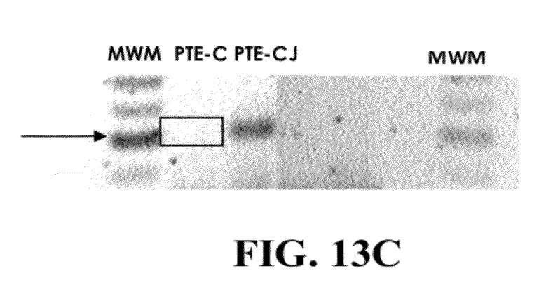

Patient clinically diagnosed with LSCD. However, the impression cytology and PAS-hematoxylin staining do not confirm the presence of goblet cells in the cornea (FIG. 13A). FIG. 13B shows that the cornea sample from the patient (PTE-C) has very low values, around zero, which are correlated with those obtained by means of agarose gel electrophoresis (FIG. 13C). Likewise, the samples analyzed by RT-PCR do not show the amplification of the specific 103 bp fragment of the mRNA of the MUC5AC gene (FIG. 13C) in the analyzed cornea.

### Case 7: Sample from Patient 0123

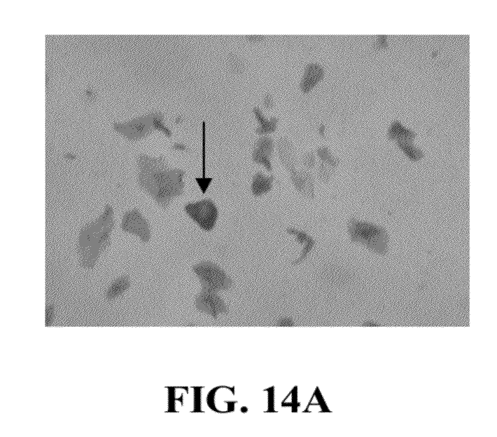

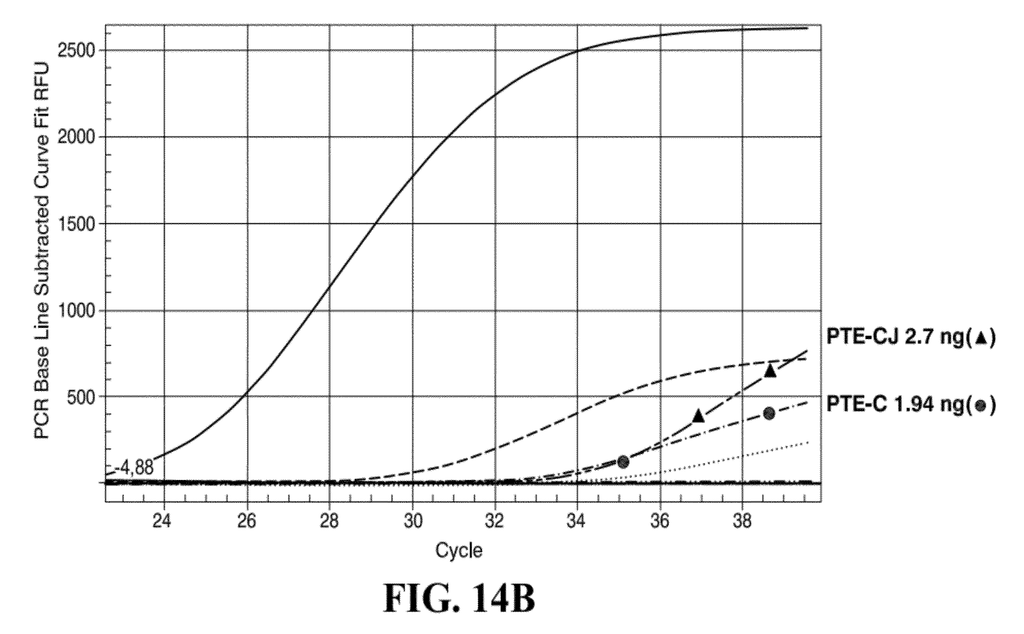

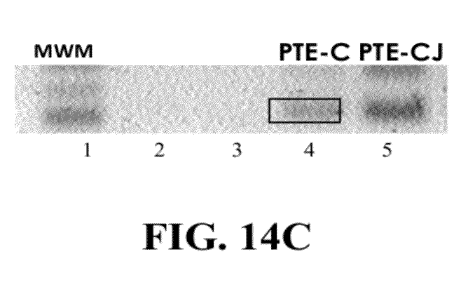

Patient clinically diagnosed with LSCD. The impression cytology and PAS-hematoxylin staining confirm the presence of goblet cells in the cornea (FIG. 14A). Likewise, LSCD is confirmed by means of RT-PCR due to the amplification of the specific 103 bp fragment of the mRNA of the MUC5AC gene (FIG. 14C). The PTE-C curve (FIG. 14B) corresponds to the cornea sample from the patient which in the gel corresponds to lane 4 (FIG. 14C). The intensity of the band is correlated with the starting amount of mRNA.

### Case 8: Sample from Patient 0125

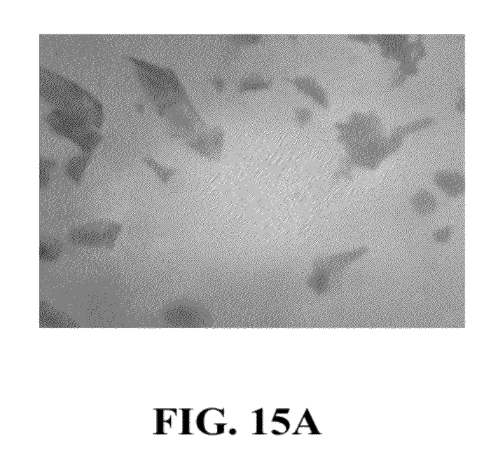

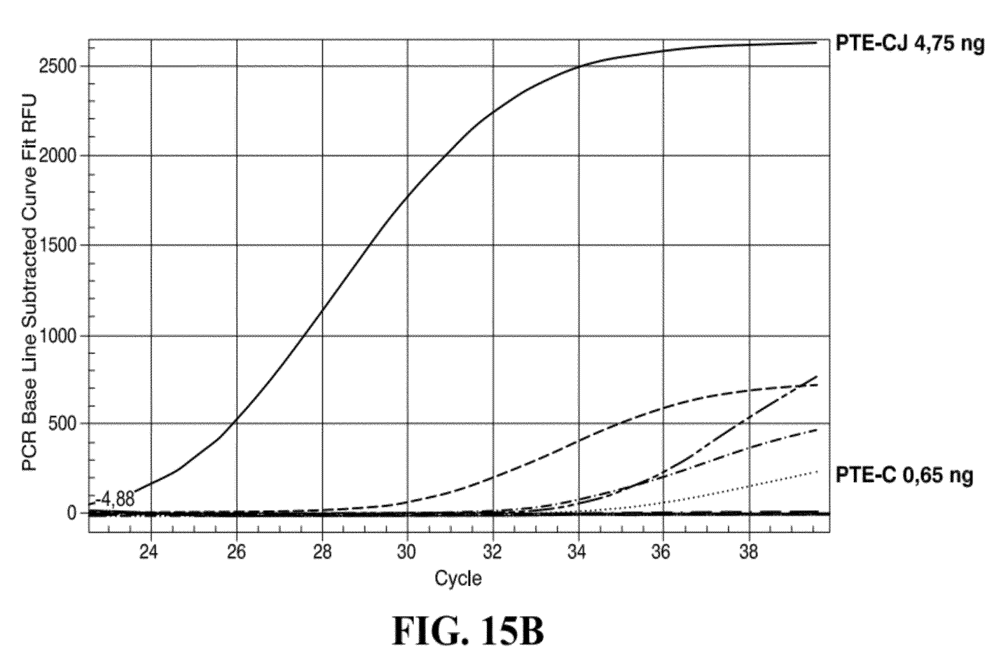

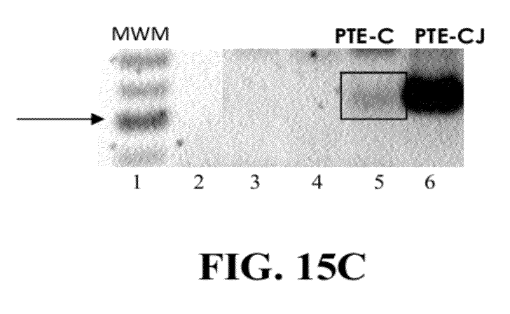

The impression cytology and PAS-hematoxylin staining do not show the presence of goblet cells in the cornea (FIG. 15A); nevertheless, the amplification by means of RT-PCR of the specific 103 bp fragment of the mRNA of the MUC5AC gene in the analyzed cornea (FIG. 15C) confirms the LSCD. The PTE-C curve (FIG. 15B) corresponds to the cornea sample from the patient which in the gel corresponds to lane 5 (FIG. 15C) and corroborates the results obtained by agarose gel electrophoresis.

### Case 9: Sample from Patient 0132

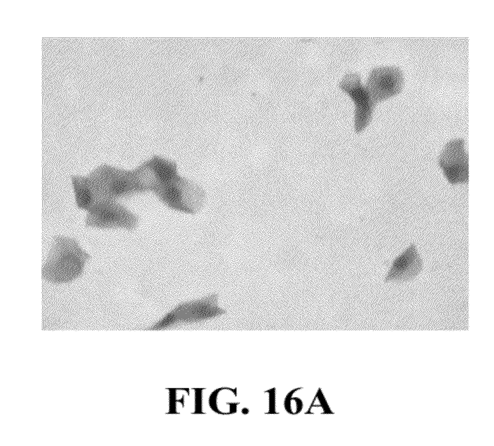

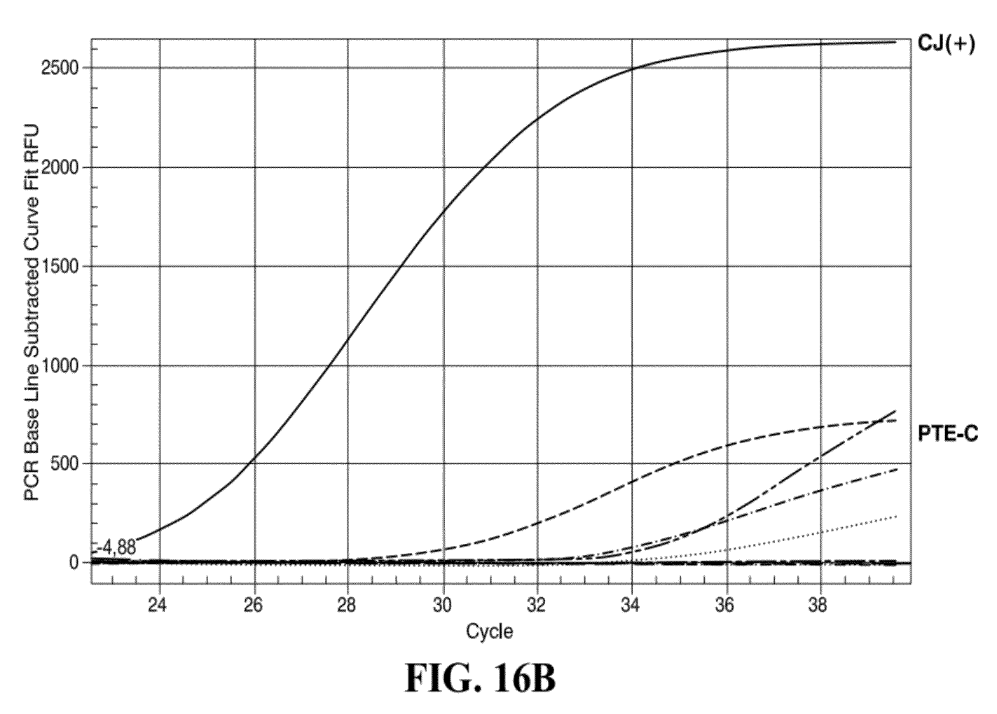

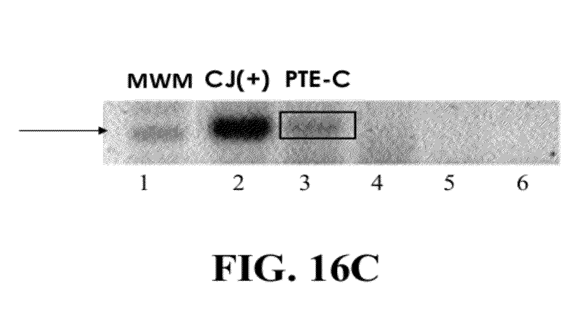

The impression cytology and PAS-hematoxylin staining do not show the presence of goblet cells in the cornea (FIG. 16A); nevertheless, the amplification by means of RT-PCR of the specific 103 bp fragment of the mRNA of the MUC5AC gene in the analyzed cornea (FIG. 16C) confirms the LSCD. The PTE-C curve (FIG. 16B) corresponds to the cornea sample from the patient which in the gel corresponds to lane 3 (FIG. 16C) and corroborates the results obtained by agarose gel electrophoresis.

### Cases 10 and 11: Samples from Patients 0172 and 0186

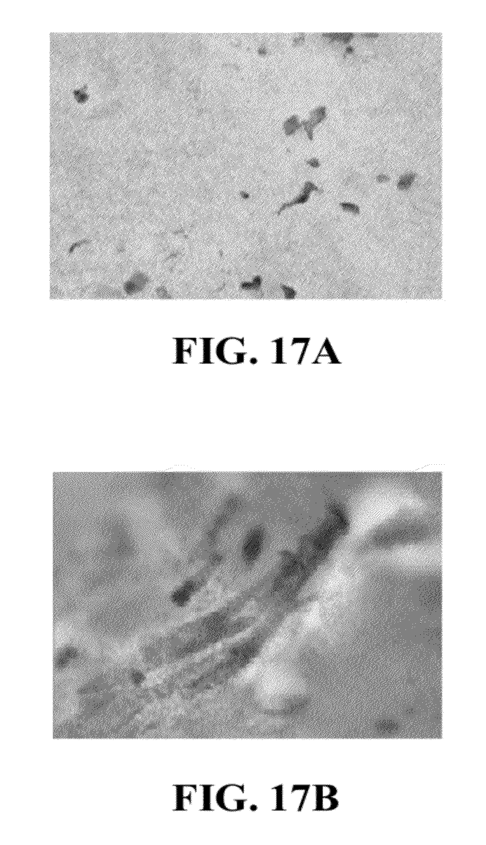

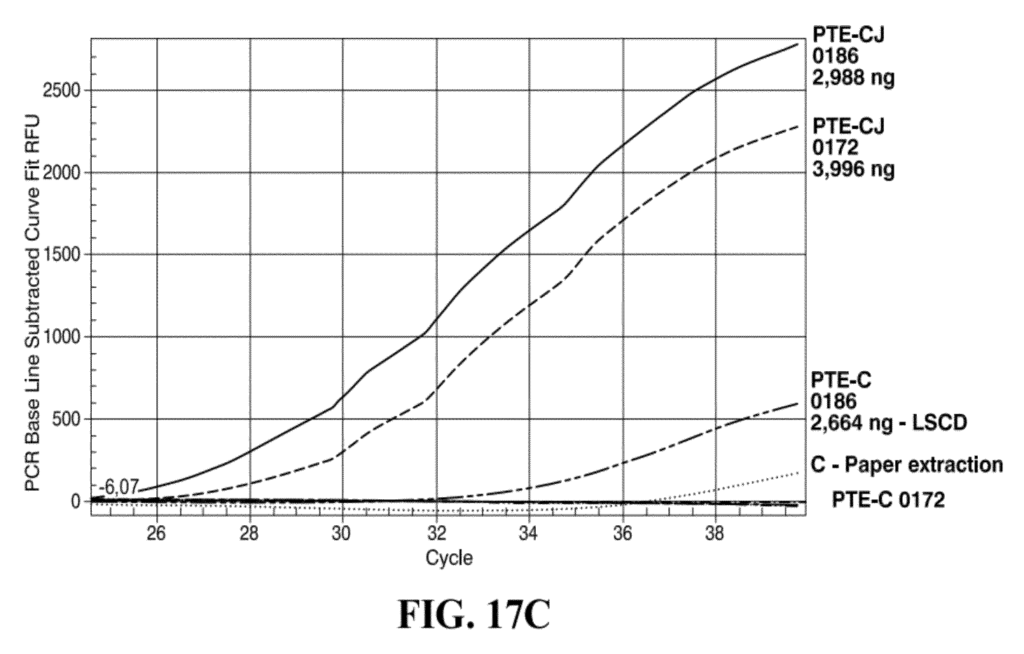

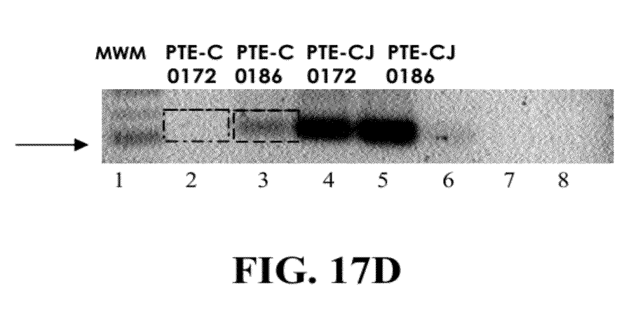

The impression cytologies and PAS-hematoxylin staining do not show the presence of goblet cells in the corneas of any of patients 0172 and 0186 (FIGS. 17A and 17B); however, the amplification by means of RT-PCR of the 103 bp fragment of the mRNA of the MUC5AC gene in the cornea of patient 0186 (FIG. 17D) confirms the LSCD in said patient. The amplification of said 103 bp fragment of the mRNA of MUC5AC in the cornea of patient 0172 is not observed (FIG. 17D), therefore a diagnosis of LSCD cannot be confirmed by means of RT-PCR. FIG. 17C shows curves of conjunctiva samples from both patients (0186 and 0172) with values above 2,000 RFU (positive samples). The cornea sample from patient 0172 gives values around zero and the cornea sample from patient 0186 (PTE-C 0186) gives low intensities, due to the small amount of mRNA.

### Case 12: Sample from Patient 0214

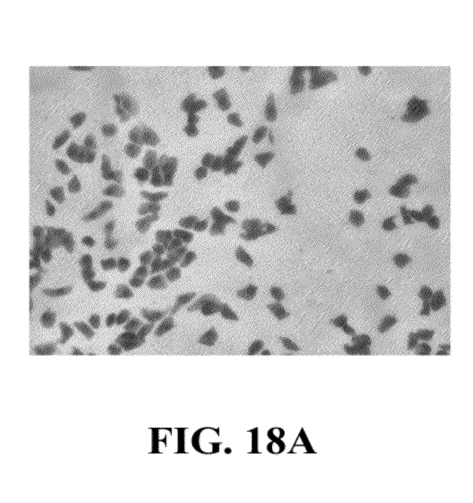

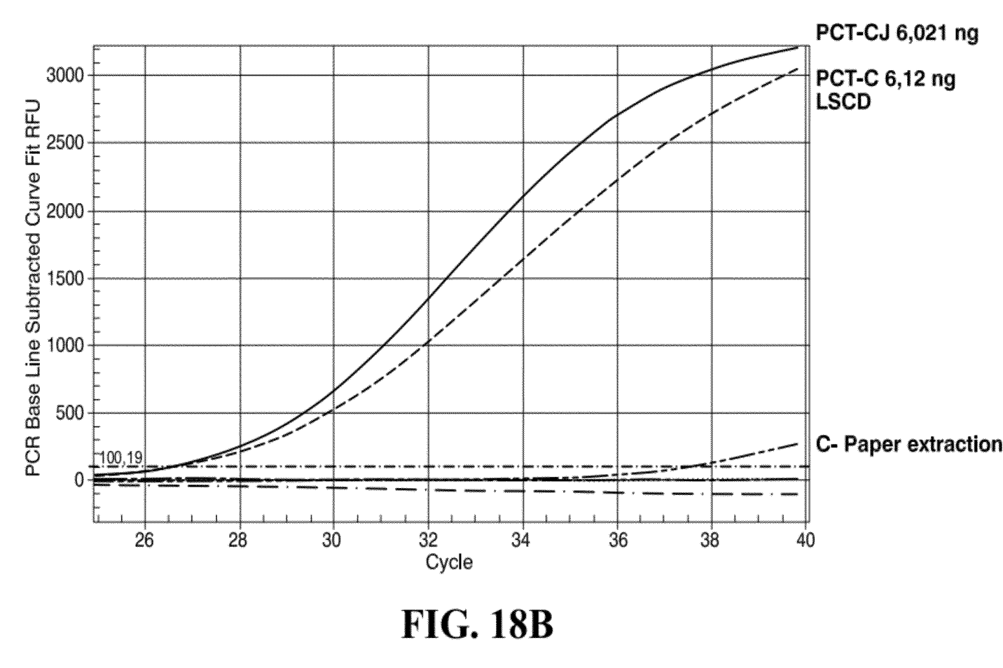

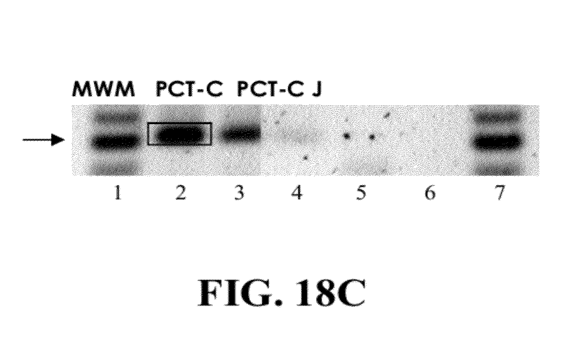

The impression cytology and PAS-hematoxylin staining do not show the presence of goblet cells in the cornea (FIG. 18A); nevertheless, the amplification by means of RT-PCR of the specific 103 bp fragment of the mRNA of the MUC5AC gene in the analyzed cornea (FIG. 18C) confirms the LSCD. In FIG. 18B, the curve corresponding to the cornea sample from the patient (PCT-C) shows values above 3,000 RFU, which indicates a very large amount of mRNA. This result is corroborated with the intensity of the band in the agarose gel.

### Case 13: Sample from Patient 0233

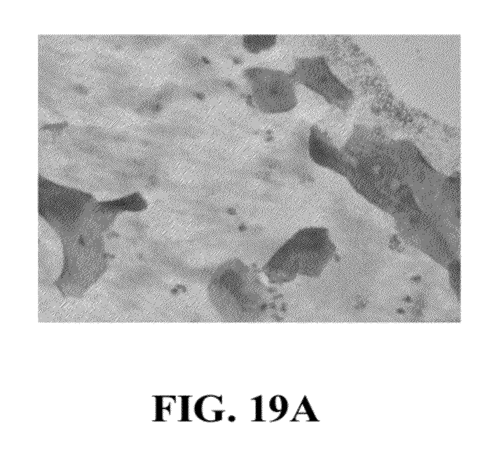

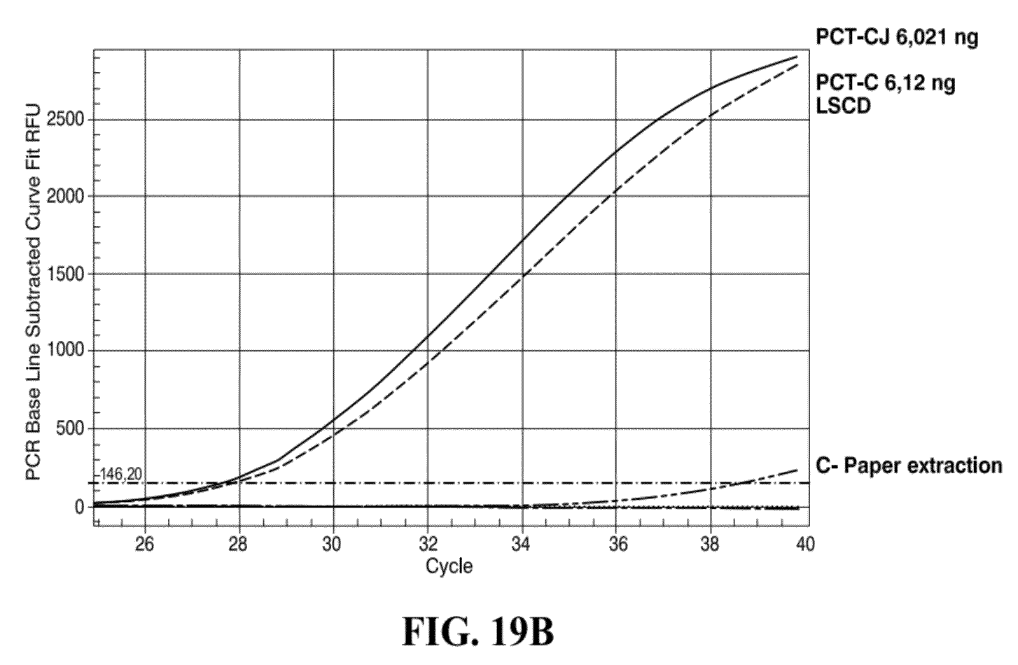

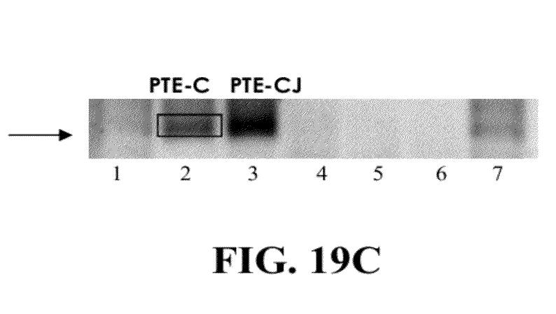

Patient clinically diagnosed with LSCD. The impression cytology and PAS-hematoxylin staining confirm the presence of goblet cells in the cornea (FIG. 19A). Likewise, the LSCD is confirmed by means of RT-PCR due to the amplification of the specific 103 bp fragment of the mRNA of the MUC5AC gene (FIG. 19C). FIG. 19B shows the two curves corresponding to the conjunctiva (PTE-CJ) and cornea (PTE-C) samples from the patient.

By way of a summary, the results obtained in cases 1-13 are shown in Table 4.

TABLE 4
Summary of the results obtained [Cases 1-13]

| PATIENTS | | PAS- | | |
|---|---|---|---|---|
| (code) | SIGNS | HEMATOXYLIN | PCR | DIAGNOSIS |
| 5 | + | No sample | + | + |
| 6 | + | No sample | − | − |
| 7 | + | No sample | − | − |
| 0216 (RE) | + | + | + | + |
| 0216 (LE) | + | + | + | + |
| 076 | + | + | + | + |
| 0121 | + | − | − | − |
| 0123 | + | + | + | + |
| 0125 | + | − | + | + |
| 0132 | + | − | + | + |
| 0172 | + | − | − | − |
| 0186 | + | − | + | + |
| 0214 | + | − | + | + |
| 0233 | + | + | + | + |

### Results of the Second Group of Patients

A second study was conducted with a higher number of patients, in order to validate the results obtained with the first group of patients.

The impression cytology samples were analyzed to obtain the LSCD diagnosis, so that the sample is considered as positive if there are goblet cells in the cornea. Cytology samples to be analyzed by PAS-hematoxylin were not obtained for all the cases analyzed in this second group. Results are shown in comparative tables of PAS-hematoxylin diagnosis with RT-PCR, indicating whether the sample is positive or negative. The conjunctiva sample is used as a positive reference sample.

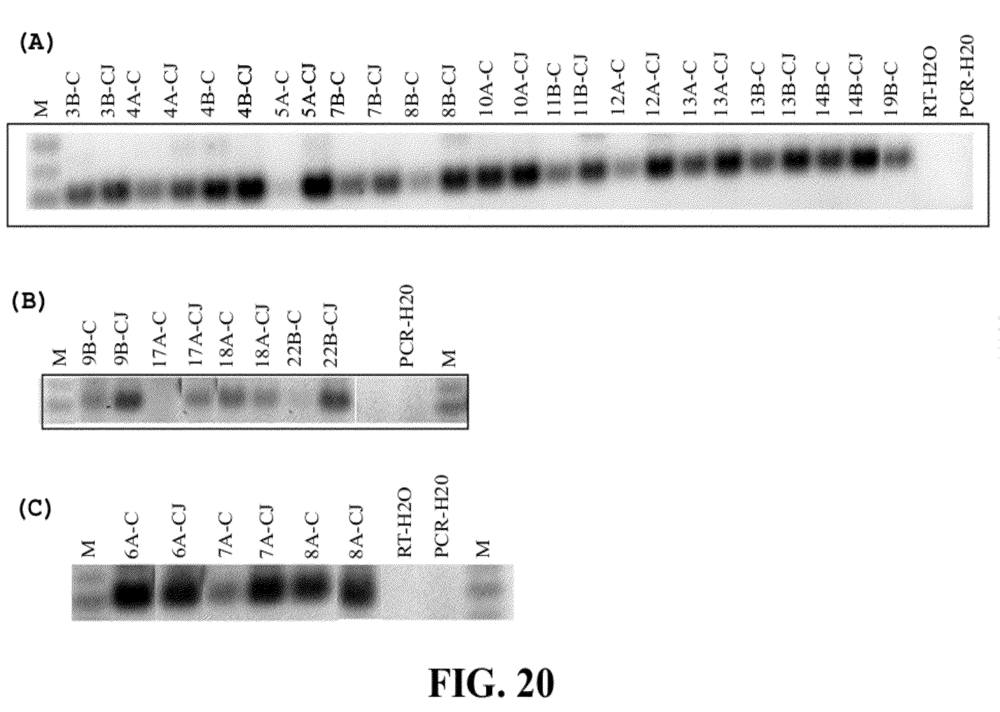

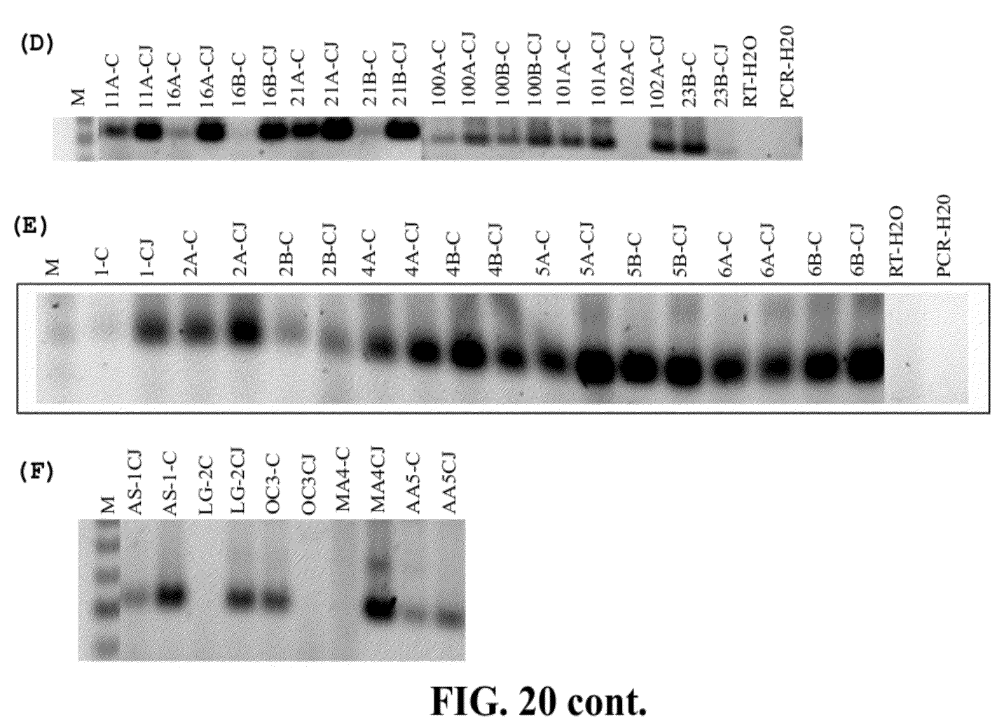

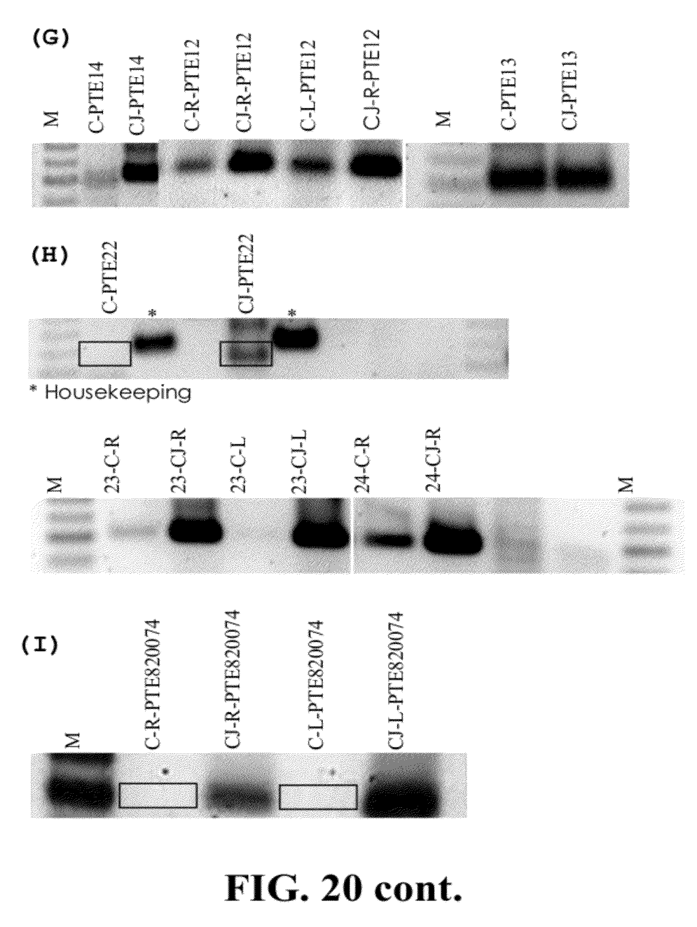

Different assays were carried out to analyze the samples by RT-PCR, that is, to analyze the amplification of the transcript of MUC5AC gene. The amplification of the transcript of the MUC5AC gene and the existence of a gel band in cornea is indicative of a patient being positive for LSCD. The results are shown in Tables 5 to 16 and in FIG. 20, wherein “C” means cornea and “CJ” conjunctiva. PCR-H2O means the PCR negative control and M the molecular weight markers. A and B indicates the right and the left eye of the patient, respectively.

TABLE 5
(corresponding to results shown in FIG. 20A)

| PATIENT | SAMPLE | RESULT |
|---|---|---|
| 3B-C | CORNEA | POSITIVE |
| 3B-CJ | CONJUNCTIVA | POSITIVE |
| 4A-C | CORNEA | POSITIVE |
| 4A-CJ | CONJUNCTIVA | POSITIVE |
| 4B-C | CORNEA | POSITIVE |
| 4B-CJ | CONJUNCTIVA | POSITIVE |
| 5A-C | CORNEA | POSITIVE |
| 5A-CJ | CONJUNCTIVA | POSITIVE |
| 7B-C | CORNEA | POSITIVE |
| 7B-CJ | CONJUNCTIVA | POSITIVE |
| 8B-C | CORNEA | POSITIVE |
| 8B-CJ | CONJUNCTIVA | POSITIVE |
| 10A-C | CORNEA | POSITIVE |
| 10A-CJ | CONJUNCTIVA | POSITIVE |
| 11B-C | CORNEA | POSITIVE |
| 11B-CJ | CONJUNCTIVA | POSITIVE |
| 12A-C | CORNEA | POSITIVE |
| 12A-CJ | CONJUNCTIVA | POSITIVE |
| 13A-C | CORNEA | POSITIVE |
| 13A-CJ | CONJUNCTIVA | POSITIVE |
| 13B-C | CORNEA | POSITIVE |
| 13B-CJ | CONJUNCTIVA | POSITIVE |
| 14B-C | CORNEA | POSITIVE |
| 14B-CJ | CONJUNCTIVA | POSITIVE |
| 19B-C | CORNEA | POSITIVE |

TABLE 6
(corresponding to results shown in FIG. 20C)

| PATIENT | SAMPLE | RESULT |
|---|---|---|
| 6A-C | CORNEA | POSITIVE |
| 6A-CJ | CONJUNCTIVA | POSITIVE |
| 7A-C | CORNEA | POSITIVE |
| 7A-CJ | CONJUNCTIVA | POSITIVE |
| 8A-C | CORNEA | POSITIVE |
| 8A-CJ | CONJUNCTIVA | POSITIVE |

TABLE 7
(corresponding to results shown in FIG. 20B)

| PATIENT | SAMPLE | RESULT |
|---|---|---|
| 9B-C | CORNEA | POSITIVE |
| 9B-CJ | CONJUNCTIVA | POSITIVE |
| 17A-C | CORNEA | NEGATIVE |
| 17A-CJ | CONJUNCTIVA | POSITIVE |
| 18A-C | CORNEA | POSITIVE |
| 18A-CJ | CONJUNCTIVA | POSITIVE |
| 22B-C | CORNEA | POSITIVE |
| 22B-CJ | CONJUNCTIVA | POSITIVE |

A comparison between the results obtained from diagnosis with PAS-hematoxylin and with RT-PCR in patients included in Tables 5, 6 and 7, is shown in Table 8.

TABLE 8
Comparison of the results obtained with PAS-hematoxylin and
RT-PCR techniques

| Patient | PAS | RT-PCR |
|---|---|---|
| 2A | − | N/A |
| 3B | − | + |
| 4A | − | + |
| 4B | + | + |
| 5A | + | + |
| 6A | − | + |
| 7A | − | + |
| 7B | − | + |
| 8A | − | + |
| 8B | − | + |
| 9B | − | + |
| 10A | − | + |
| 11B | − | + |
| 12A | + | + |
| 13A | − | + |
| 13B | − | + |
| 14B | − | + |
| 15A | − | N/A |
| 16A | − | N/A |
| 17A | − | − |
| 18A | − | + |
| 19B | − | + |
| 20B | − | N/A |
| 21A | + | N/A |
| 22B | + | + |

25 eye samples corresponding to 18 patients were analyzed. According to the results obtained by RT-PCR, 19 of the samples were positive, 1 was negative and 5 samples could not be analyzed (N/A), because the amount of RNA in the sample was not enough to perform the analysis. The comparative analysis between the molecular diagnosis and the cytological diagnosis indicates that 5 of the samples analyzed in the assay were coincident in the result obtained in the diagnosis with PAS-hematoxylin and RT-PCR.

In the following table (Table 9), 20 more samples corresponding to 10 patients were analyzed in cornea and conjunctiva and in Table 10 a comparison between the cytological diagnosis and the RT-PCR diagnosis of some of the samples is shown.

TABLE 9
(corresponding to FIG. 20D)

| PATIENT | SAMPLE | RESULT |
|---|---|---|
| 11A-C | CORNEA | POSITIVE |
| 11A-CJ | CONJUNCTIVA | POSITIVE |
| 16A-C | CORNEA | POSITIVE |
| 16A-CJ | CONJUNCTIVA | POSITIVE |
| 16B-C | CORNEA | NEGATIVE |
| 16B-CJ | CONJUNCTIVA | POSITIVE |
| 21A-C | CORNEA | POSITIVE |
| 21A-CJ | CONJUNCTIVA | POSITIVE |
| 21B-C | CORNEA | POSITIVE |
| 21B-CJ | CONJUNCTIVA | POSITIVE |
| 100A-C | CORNEA | POSITIVE |
| 100A-CJ | CONJUNCTIVA | POSITIVE |
| 100B-C | CORNEA | POSITIVE |
| 100B-CJ | CONJUNCTIVA | POSITIVE |
| 101A-C | CORNEA | POSITIVE |
| 101A-CJ | CONJUNCTIVA | POSITIVE |
| 102A-C | CORNEA | NEGATIVE |
| 102A-CJ | CONJUNCTIVA | POSITIVE |
| 23B-C | CORNEA | POSITIVE |
| 23B-CJ | CONJUNCTIVA | POSITIVE |

The results shown in Table 9 indicate that the cytological diagnosis is confirmed by RT-PCR in 8 patients as positive and in 2 patients as negative. The comparison between both techniques shown in Table 10 indicates that there are coincident results in 3 of the cases analyzed.

TABLE 10
Comparison of the results obtained with PAS-hematoxylin and
RT-PCR techniques

| Patient | PAS | RT-PCR |
|---|---|---|
| 11A | − | + |
| 16A | + | + |
| 16B | +? | − |
| 21A | + | + |
| 21B | + | + |
| 100A | − | + |
| 100B | +? | + |
| 101A | − | + |
| 102A | | − |
| 23B | | + |

New samples from 9 patients were analyzed, only by RT-PCR and not by PAS-hematoxylin. The results are shown in Table 11.

TABLE 11
(corresponding to FIG. 20E)

| PATIENT | SAMPLE | RESULT |
|---|---|---|
| 1-C | CORNEA | POSITIVE |
| 1-CJ | CONJUNCTIVA | POSITIVE |
| 2A-C | CORNEA | POSITIVE |
| 2A-CJ | CONJUNCTIVA | POSITIVE |
| 2B-C | CORNEA | POSITIVE |
| 2B-CJ | CONJUNCTIVA | POSITIVE |
| 4A-C | CORNEA | POSITIVE |
| 4A-CJ | CONJUNCTIVA | POSITIVE |
| 4B-C | CORNEA | POSITIVE |
| 4B-CJ | CONJUNCTIVA | POSITIVE |
| 5A-C | CORNEA | POSITIVE |
| 5A-CJ | CONJUNCTIVA | POSITIVE |
| 5B-C | CORNEA | POSITIVE |
| 5B-CJ | CONJUNCTIVA | POSITIVE |
| 6A-C | CORNEA | POSITIVE |
| 6A-CJ | CONJUNCTIVA | POSITIVE |
| 6B-C | CORNEA | POSITIVE |
| 6B-CJ | CONJUNCTIVA | POSITIVE |

New samples from 5 patients were analyzed by RT-PCR and by PAS-hematoxylin. The results are shown in Table 12.

TABLE 12
(corresponding to FIG. 20F)

| PATIENT | SAMPLE | RESULT |
|---|---|---|
| AS-1 | CORNEA | POSITIVE |
| AS-1 | CONJUNCTIVA | POSITIVE |
| LG-2 | CORNEA | NEGATIVE |
| LG-2 | CONJUNCTIVA | POSITIVE |
| OC-3 | CORNEA | POSITIVE |
| OC-3 | CONJUNCTIVA | NEGATIVE* |
| MA-4 | CORNEA | NEGATIVE |
| MA-4 | CONJUNCTIVA | POSITIVE |
| AA-5 | CORNEA | POSITIVE |
| AA-5 | CONJUNCTIVA | POSITIVE |

According to the results obtained by RT-PCR, 3 of the samples were positive and 2 were negative. Using the PAS-hematoxylin diagnosis, two of the samples could not be analyzed due to the low amount of sample. The comparative analysis between the molecular diagnosis and the cytological diagnosis indicates that 2 of the samples analyzed in the assay were coincident in the result obtained in the diagnosis with PAS-hematoxylin and RT-PCR (Table 13).

TABLE 13
Comparison of the results obtained with RT-PCR
and PAS-hematoxylin techniques

| PATIENTS | RT-PCR | PAS-HEMATOXYLIN |
|---|---|---|
| AS-1 | POSITIVE | POSITIVE |
| LG-2 | NEGATIVE | NOT ENOUGH SAMPLE |
| OC-3 | POSITIVE | POSITIVE |
| MA-4 | NEGATIVE | NOT ENOUGH SAMPLE |
| AA-5 | POSITIVE | NEGATIVE |

New samples were analyzed and results are shown in Tables 14, 15 and 16. The PAS-hematoxylin diagnosis was not performed for Patients 14 (Pte 14) and 820074, the one performed for Patient 12 (Pte 12) was not conclusive and the result for Patients 13 and 22 (Pte 13 and Pte 22) was negative, which is not the same as the result as the one obtained for the RT-PCR.

Results of the RT-PCR of Patient 23, from the right eye (R) and the left eye (L) are shown in Table 15 and FIG. 20H. The diagnosis by PAS-hematoxylin has not been performed for this patient. The sample from Patient 24 comes from the right eye and the result is positive. No results from PAS-hematoxylin diagnosis has been performed for this patient.

TABLE 14
(corresponding to FIG. 20G)

| PATIENT | SAMPLE | RESULT |
|---|---|---|
| Pte 14 | CORNEA | POSITIVE |
| Pte 14 | CONJUNCTIVA | POSITIVE |
| Pte 12-OD | CORNEA | POSITIVE |
| Pte 12-OD | CONJUNCTIVA | POSITIVE |
| Pte 12-OI | CORNEA | POSITIVE |
| Pte 12-OI | CONJUNCTIVA | POSITIVE |
| Pte 13 | CORNEA | POSITIVE |
| Pte 13 | CONJUNCTIVA | POSITIVE |

TABLE 15
(corresponding to FIG. 20H)

| PATIENT | SAMPLE | RESULT |
|---|---|---|
| Pte 22 | CORNEA | NEGATIVE |
| Pte 22 | CONJUNCTIVA | POSITIVE |
| Pte 23-right | CORNEA | POSITIVE |
| Pte 23-right | CONJUNCTIVA | POSITIVE |
| Pte 23-left | CORNEA | POSITIVE* |
| Pte 23-left | CONJUNCTIVA | POSITIVE |
| Pte 24-right | CORNEA | POSITIVE |
| Pte 24-right | CONJUNCTIVA | POSITIVE |

TABLE 16
(corresponding to FIG. 20I)

| PATIENT | SAMPLE | RESULT |
|---|---|---|
| Pte 820074-OD | CORNEA | NEGATIVE |
| Pte 820074-OD | CONJUNCTIVA | POSITIVE |
| Pte 820074-OI | CORNEA | NEGATIVE |
| Pte 820074-OI | CONJUNCTIVA | POSITIVE |

### CONCLUSIONS

As it can be seen, the correlation is greater between the clinical diagnosis and the impression cytology analyzed by RT-PCR than between the clinical diagnosis and the impression cytology stained with PAS-hematoxylin. The sensitivity and the specificity of the described technique (RT-PCR) are greater than those of the conventional impression cytology stained with PAS-hematoxylin.

There are 4 manifestations of LSCD in the first group of patients which are clinically but not molecularly diagnosed. In these cases the medical record of the studied patients was reviewed, suspecting an erroneous clinical diagnosis.

In the first group of patients, by means of impression cytology stained with PAS-hematoxylin, LSCD could only be diagnosed in 5 cases of the 9 cases analyzed by the two methods and which were positive [patients 0216 (2 eyes), 076, 0123 and 0244]. There are 4 cases which, analyzed by means of impression cytology stained with PAS-hematoxylin, can be considered as “false negatives” since they were negative by means of that technique but positive by means of RT-PCR, resulting in an erroneous diagnosis [patients 0125, 032, 0186 and 0214].

Furthermore, in the second group of patients, by means of impression cytology stained with PAS-hematoxylin, LSCD could only be diagnosed in 10 cases of the 40 cases analyzed by the two methods and which were positive. There are 20 cases which, analyzed by means of impression cytology stained with PAS-hematoxylin, can be considered as “false negatives” since they were negative by means of that technique but positive by means of RT-PCR, resulting in an erroneous diagnosis.

In view of these results, the success of the diagnosis of LSCD by PCR (sensitivity of the technique), can be estimated at close to 100% in the analyzed cases, compared to 45% of the technique based on PAS-hematoxylin staining.

1. An in vitro method for diagnosing limbal stem cell deficiency (LSCD) in a subject, comprising:
analyzing the expression of the MUC5AC gene in a sample of cornea from said subject, using a pair of oligonucleotide primers under conditions which allow specifically amplifying a fragment of the MUC5AC transcript, said fragment having at least 75 nucleotides long and being included within a region consisting of nucleotides 17440-18750 in the MUC5AC cDNA nucleotide sequence shown in SEQ ID NO: 1, wherein the detection of the expression of the MUC5AC gene in said sample of cornea is indicative of LSCD;
or alternatively
a) detecting the expression of the MUC5AC gene in a sample cornea from said subject; and
b) comparing the expression level of the MUC5AC gene detected in step a) with the expression level of the MUC5AC gene in a reference sample;
wherein step a) comprises detecting the expression of the MUC5AC gene in a cornea sample from said subject, using a pair of oligonucleotide primers under conditions which allow specifically amplifying a fragment of the MUC5AC transcript, said fragment being at least 75 nucleotides long and being included within a region consisting of nucleotides 17440-18750 in the MUC5AC cDNA nucleotide sequence shown in SEQ ID NO: 1; and
wherein an increase in the expression level of the MUC5AC gene detected in step a) with respect to the expression level of the MUC5AC gene in the reference sample is indicative of LSCD.

2. The method according to claim 1, wherein step a) comprises detecting the expression of the MUC5AC gene in a cornea sample from said subject, using a pair of oligonucleotide primers formed by an oligonucleotide primer comprising the nucleotide sequence identified as SEQ ID NO: 2 and by an oligonucleotide primer comprising the nucleotide sequence identified as SEQ ID NO: 3, under conditions which allow amplifying a specific fragment of the MUC5AC transcript.

3. The method according to claim 1, wherein step a) comprises detecting the expression of the MUC5AC gene by means of RT-PCR.

4. The method according to claim 1, wherein said reference sample is a healthy cornea sample.

5. An in vitro method for diagnosing limbal stem cell deficiency (LSCD) in a subject, comprising
analyzing the expression of the MUC5AC gene in a cornea sample from said subject by an amplification reaction, using a pair of oligonucleotide primers consisting of an oligonucleotide primer comprising the nucleotide sequence identified as SEQ ID NO: 2 and an oligonucleotide primer comprising the nucleotide sequence identified as SEQ ID NO: 3, under conditions which allow amplifying a specific fragment of the MUC5AC gene, wherein the detection of the expression of the MUC5AC gene in said sample of cornea is indicative of LSCD;
or alternatively
a) detecting the expression of the MUC5AC gene in a cornea sample from said subject by an amplification reaction, using a pair of oligonucleotide primers consisting of an oligonucleotide primer comprising the nucleotide sequence identified as SEQ ID NO: 2 and an oligonucleotide primer comprising the nucleotide sequence identified as SEQ ID NO: 3, under conditions which allow amplifying a specific fragment of the MUC5AC gene; and
b) comparing the expression level of the MUC5AC gene detected in step (a) with the expression level of the MUC5AC gene in a reference sample;
wherein an increase in the expression level of the MUC5AC gene detected in step (a) with respect to the expression level of the MUC5AC gene in the reference sample is indicative of LSCD.

6. An oligonucleotide selected from the group of oligonucleotides consisting of an oligonucleotide comprising the nucleotide sequence shown in SEQ ID NO: 2, an oligonucleotide comprising the nucleotide sequence shown in SEQ ID NO: 3 and combinations of both oligonucleotides.

7. A kit comprising at least one oligonucleotide according to claim 6, or a pair of oligonucleotide primers consisting of an oligonucleotide primer comprising the nucleotide sequence shown in SEQ ID NO: 2 and an oligonucleotide primer comprising the nucleotide sequence shown in SEQ ID NO: 3.

8. The kit according to claim 7, further comprising a substance labeled with a fluorophore.

9. The kit according to claim 8, wherein said substance labeled with a fluorophore comprises a specific Taqman type probe suitable for detecting the MUC5AC transcript.

10. (canceled)

11. A method for detecting and identifying the MUC5AC transcript, or for quantifying the expression of said MUC5AC gene, or for the diagnosis of LSCD by means of detecting and/or quantifying said transcript of the MUC5AC gene, said method comprising using a pair of oligonucleotides selected from the group consisting of an oligonucleotide comprising the nucleotide sequence shown in SEQ ID NO: 2 and an oligonucleotide comprising the nucleotide sequence shown in SEQ ID NO: 3, as oligonucleotide primers in a PCR.

12. A method for evaluating if a patient with a corneal pathology susceptible to keratoplasty is a suitable candidate for keratoplasty, comprising:
detecting the expression of the MUC5AC gene in a cornea sample from said patient; or, alternatively
determining the expression level of the MUC5AC gene in a cornea sample from said patient; and
if the expression of the MUC5AC gene is not detected in said cornea sample from said patient; or, alternatively, if the expression level of the MUC5AC gene detected in the cornea sample from said patient is equal to the expression level of the MUC5AC gene in a reference sample, then said patient is a candidate suitable for corneal transplantation or keratoplasty.

13. The method according to claim 2, wherein step a) comprises detecting the expression of the MUC5AC gene by means of RT-PCR.


SEQUENCE LISTING (20 sequences)

SEQ ID NO: 1 - DNA, 18750 bp, Homo sapiens
atgtcgctgcagttcaatgggtttgtgcgttgtgatcacacggtcctgcccacagagctgaatgggctattggggtttccttggaggctgctgagggacagggcactcttccccgccgtccacacaatgagtgttggccggaggaagctggccctgctctgggccctggctctcgctctggcctgcacccggcacacaggccatgcccaggatggctcctccgaatccagctacaagcaccaccctgccctctctcctatcgcccgggggcccagcggggtcccgctccgtggggcgactgtcttcccatctctgaggaccatccctgtggtacgagcctccaacccggcgcacaacgggcgggtgtgcagcacctggggcagcttccactacaagaccttcgacggcgacgtcttccgcttccccggcctctgcaactacgtgttctccgagcactgcggtgccgcctacgaggattttaacatccagctacgccgcagccaggagtcagcggcccccacgctgagcagggtcctcatgaaggtggatggcgtggtcatccagctgaccaagggctccgtcctggtcaacggccacccggtcctgctgcccttcagccagtctggggtcctcattcagcagagcagcagctacaccaaggtggaggccaggctgggccttgtcctcatgtggaaccacgatgacagcctgctgctggagctggacaccaaatacgccaacaagacctgtgggctctgtggggacttcaacgggatgcccgtggtcagcgagctcctctcccacaacaccaagctgacacccatggaattcgggaacctgcagaagatggacgaccccacggagcagtgtcaggaccctgtccctgaacccccgaggaactgctccactggctttggcatctgtgaggagctcctgcacggccagctgttctctggctgcgtggccctggtggacgtcggcagctacctggaggcttgcaggcaagacctctgcttctgtgaagacaccgacctgctcagctgcgtctgccacacccttgccgagtactcccggcagtgcacccatgcaggggggttgccccaggactggcggggccctgacttctgcccccagaagtgccccaacaacatgcagtaccacgagtgccgctccccctgcgcagacacctgctccaaccaggagcactcccgggcctgtgaggaccactgtgtggccggctgcttctgccctgaggggacggtgcttgacgacatcggccagaccggctgtgtccctgtgtcaaagtgtgcctgcgtctacaacggggctgcctatgccccaggggccacctactccacagactgcaccaactgcacctgctccggaggccggtggagctgccaggaggttccatgcccgggtacctgctctgtgcttggaggtgcccacttctcaacgtttgacgggaagcaatacacggtgcacggcgactgcagctatgtgctgaccaagccctgtgacagcagtgccttcactgtactggctgagctgcgcaggtgcgggctgacggacagcgagacctgcctgaagagcgtgacactgagcctggatggggcgcagacggtggtggtgatcaaggccagtggggaagtgttcctgaaccagatctacacccagctgcccatctctgcagccaacgtcaccatcttcagaccctcaaccttcttcatcatcgcccagaccagcctgggcctgcagctgaacctgcagctggtgcccaccatgcagctgttcatgcagctggcgcccaagctccgtgggcagacctgcggtctctgtgggaacttcaacagcatccaggccgatgacttccggaccctcagtggggtggtggaggccaccgctgcggccttcttcaacaccttcaagacccaggccgcctgccccaacatcaggaacagcttcgaggacccctgctctctgagcgtggagaatgcccccaggatgggtgccccgagcgcgtgccggacgctggtgttggctctggcggccatgctcgtggtgccgcaggcagagacccagggccctgtggagccgagctgggagaatgcagggcacaccatggatggcggtgccccgacgtcctcgcccacccggcgcgtgagctttgttccacccgtcactgtcttccccagcctgagccgtaagcagatgctgcccctgccagccgggaagggggtgtttgccagtcccaaaggtgggggcccagatctaggggtgcagctgccaccagccctgaacccggcgcacaatgggcgggtgtgcagcacctggggtgacttccactacaagaccttcgacggcgacgtcttccgcttccctggcctttgcaactacgtgttctctgagcactgccgcgccgcctacgaggacttcaacgtccagctacgccgaggcctagtgggctccaggcctgtggtcacccgtgttgtcatcaaggcccaggggctggtgctggaggcgtccaacggctccgtcctcatcaatgggcagcgggaggagctgccttacagccgcactggcctcctggtggagcagagcggggactacatcaaggtcagcatccggctggtgctgacattcctgtggaacggagaggacagtgccctgctggagctggatcccaaatacgccaaccagacctgtggcctgtgtggggacttcaacggcctcccggccttcaacgagttctatgcccacaacgccaggctgaccccgctccagtttgggaacctgcagaagttggatgggcccacggagcagtgcccggacccgctgcccttgccggccggcaactgcacggacgaggagggcatctgccaccgcaccctgctggggccggcctttgcggagtgccacgcactggtggacagcactgcgtacctggccgcctgcgcccaggacctgtgccgctgccccacctgcccgtgtgccacctttgtggaatactcacgccagtgcgcccacgcggggggccagccgcggaactggaggtgccctgagctctgcccccggacctgccccctcaacatgcagcaccaggagtgtggctcaccctgcacggacacctgctccaacccccagcgcgcgcagctctgcgaggaccactgtgtggacggctgcttctgccccccaggcaggtcttgcacggtgctggatgacatcacgcactctggctgcctgcccctcgggcagtgcccctgcacccacggcggccgcacctacagcccgggcacctccttcaacaccacctgcagctcctgcacctgctccggggggctatggcagtgccaggacctgccgtgccctggcacctgctctgtgcagggcggggcccacatctccacctatgatgagaaactctacgacctgcatggtgactgcagctacgttctgtccaagaaatgtgccgacagcagcttcaccgtgctggctgagctgcggaagtgcggcctgacggacaacgagaactgcctgaaagcggtgacgctcagcctggacggcggggacacggccatccgggtccaagcggacggcggcgtgttcctcaactccatctacacgcagctgcccctgtcggcagccaacatcaccctgttcacaccctcgagcttcttcatcgtggtgcagacaggcctcgggctgcagctgctggtgcagctggtgccactcatgcaggtgtttgtcaggctggaccccgcccaccagggccagatgtgcggcctgtgtgggaacttcaaccagaaccaggctgacgacttcacggccctcagcggggtggtggaggccacgggcgcagccttcgccaacacctggaaggcccaggctgcctgtgccaatgccaggaacagctttgaggacccctgctccctcagtgtggagaatgagaactacgcccggcactggtgctcgcgcctgaccgatcccaacagtgccttctcgcgctgccactccatcatcaaccccaagcccttccactcgaactgcatgtttgacacctgcaactgtgagcggagcgaggactgcctgtgcgccgcgctgtcctcctacgtgcacgcctgtgccgccaagggcgtacagctcagcgactggagggacggcgtctgcaccaagtacatgcagaactgccccaagtcccagcgctacgcctacgtggtggatgcctgccagcccacttgccgcggcctgagtgaggccgacgtcacctgcagcgtttccttcgtgcctgtggacggctgcacctgccccgcgggcaccttcctcaatgacgcgggcgcctgtgtgcccgcccaggagtgcccctgctacgctcacggcaccgtgctggctcctggagaggtggtgcacgacgagggcgccgtgtgttcatgtacgggtgggaagctaagctgcctgggagcctctctgcagaaaagcacagggtgtgcagcccccatggtgtacctggactgcagcaacagctcggcgggcacccctggggccgagtgcctccggagctgccacacgctggacgtgggctgtttcagcacacactgcgtgtccggctgtgtctgtcccccggggctggtgtcggatgggagtgggggctgcattgccgaggaggactgcccctgtgtgcacaacgaggccacctacaagcctggagagaccatcagggtcgactgcaacacctgcacctgcaggaaccggaggtgggagtgcagccaccggctctgcctgggcacctgcgtggcctacggggatggccacttcatcacctttgatggcgatcgctacagctttgaaggcagctgcgagtacatcttggcccaggactactgtggggacaacaccacccacgggaccttccgcatcgtcaccgagaacatcccctgtgggaccaccggcaccacctgctccaaggccatcaagctcttcgtggaggtgagaacggccccagctgtgagcacccccgaccctgcagccaacgagccggcccccagggaagcttcggtcggcttccggcagcgtctgcctcccctgcagagctacgagctgatcctccaagaggggacctttaaggcggtggcgagagggccgggtggggacccaccctacaagatacgctacatggggatcttcctggtcatcgagacccacgggatggccgtgtcctgggaccggaagaccagcgtgttcatccgactgcaccaggactacaagggcagggtctgcggcctgtgcgggaacttcgacgacaatgccatcaatgactttgccacgcgtagccggtccgtggtgggggacgcactggagtttgggaacagctggaagctctccccctcctgcccggacgccctggcacccaaggacccctgcacggccaaccccttccgcaagtcctgggcccagaagcagtgcagcatcctccacggccccaccttcgccgcctgccgctcccaggttgactccaccaagtactacgaggcctgcgtgaacgacgcgtgtgcctgcgactcgggtggcgactgcgagtgtttctgcacggctgtggctgcctacgcccaggcctgccacgacgcgggcctgtgtgtgtcctggcggactccggacacctgccccttgttctgtgacttctacaacccacatgggggctgtgagtggcactaccagccctgcggggcaccctgcctaaaaacctgccggaaccccagtgggcactgcctggtggacctgcctggcctggaaggctgctacccgaagtgcccacccagccagcccttcttcaatgaggaccagatgaagtgcgtggcccagtgtggctgctacgacaaggacggaaactactatgacgtcggtgcaagggtccccacagcggagaactgccagagctgtaactgcacacccagtggcatccagtgcgctcacagccttgaggcctgcacctgcacctatgaggacaggacctacagctaccaggacgtcatctacaacaccaccgatgggcttggcgcctgcttgatcgccatctgcggaagcaacggcaccatcatcaggaaggctgtggcatgtcctggaactccagccacaacgccattcaccttcaccaccgcctgggtcccccactccacgacaagcccggccctcccggtctccaccgtgtgtgtccgcgaggtctgccgctggtccagctggtacaatgggcaccgcccagagcccggcctgggaggcggagactttgagacgtttgaaaacctgaggcagagagggtaccaggtatgccctgtgctggctgacatcgagtgccgggcggcgcagcttcccgacatgccgctggaggagctgggccagcaggtggactgtgaccgcatgcgggggctgatgtgcgccaacagccaacagagtcccccgctctgtcacgactacgagctgcgggttctctgctgcgaatacgtgccctgtggcccctccccggccccaggcaccagccctcagccctccctcagtgccagcacggagcctgctgtgcctaccccaacccagaccacagcaaccgaaaagaccaccctatgggtgaccccgagcatccggtcgacggcggccctcacctcgcagactgggtccagctcaggccccgtgacggtcaccccctcggccccaggtaccaccacctgccagccccggtgtcagtggacagagtggtttgatgaggactaccccaagtctgaacaacttggaggggacgttgagtcctacgataagatcagggccgctggagggcacttatgccagcagcctaaggacatagagtgccaggccgagagcttccccaactggaccctggcacaggtggggcagaaggtgcactgtgacgtccacttcggcctggtgtgcaggaactgggagcaggagggcgtcttcaagatgtgctacaactacaggatccgggtcctctgctgcagtgacgaccactgcaggggacgtgccacaaccccgccaccgaccacagagctggagacggccaccaccaccaccacccaggccctgttctcaacgccgcagcctacgagtagcccggggctgaccagggctcccccggccagcaccacagcagtccccaccctctcagaaggactgacatcccccagatacacaagcacccttggtacagccaccacgggaggccccacgacgcctgcaggctccacagaacccactgtcccaggggtggccacatccacccttccaacacgctcagcccttccagggacgacggggagcttgggcacatggcgcccctcacagccacccacgctggccccaacaacaatggcaacctccagagctcgcccgacaggcacagccagcaccgcttccaaagagccgctgaccacgagcctggcgccaacactcacgagcgagctgtccacctctcaggccgagaccagcacgcccaggacagagacgacaatgagccccttgactaacaccaccaccagccagggcacgacccgctgtcaaccgaagtgtgagtggacagagtggtttgacgtggacttcccaacctcaggggttgcaggcggggacatggaaacttttgaaaacatcagggctgctgggggcaagatgtgctgggcaccaaagagcatagagtgccgggcggagaactaccccgaggtaagcatcgaccaggtcgggcaggtgctgacctgcagcctggagacggggctgacctgcaagaacgaagaccagacaggcaggttcaacatgtgcttcaactacaacgtgcgtgtgctttgctgtgacgactacagccactgccccagtaccccagccaccagctccacggccacgccctcctcaactccggggacgacctggatcctcacaaagccgaccacaacagccactacgactgcgtccactggatccacggccaccccgacctccaccctgagaacagctccccctcccaaagtgctgaccaccacggccaccacacccacagtcaccagctccaaagccactccctcctccagtccagggactgcaaccgcccttccagcactgagaagcacagccaccacacccacagctaccagcgttacacccatcccctcttcctccctgggcaccacctggacccgcctatcacagaccaccacacccacggccaccatgtccacagccacaccctcctccactccagagactgcccacacctccacagtgcttaccgccacggccaccacaactggggccaccggctctgtggccaccccctcctccaccccaggaacagctcacactaccaaagtgccaactaccacaaccacgggcttcacagccaccccctcctccagcccagggacggcactcacgcctccagtgtggatcagcacaaccaccacacccacaaccagaggctccacggtgaccccctcctccatcccggggaccacccacaccgccacagtgctgaccaccaccaccacaactgtggccactggttctatggcaacaccctcctctagcacacagaccagtgggaccacccacacacccccagtgccgaacaccatggccaccacacacgggcgatccctgccccccagcagtccccacacggtgcgcacagcctggacttcggccacctcgggcatcttgggcaccacccacatcacagagccttccacggtgacttcccacaccctagcagcaaccaccggtaccacccagcactcgactccagccctttccagccctcaccctagcagcagaaccaccgagtcacccccttctccagggacgaccaccccgggccacaccacggccacctccaggaccacagccacggccacacccagcaagacccgcacctcgaccctgctgcccagcagccccacatcggcccccataaccacggtggtgaccatgggctgtgagccccagtgtgcctggtcagagtggctggactacagctaccccatgccggggccctctggcggggactttgacacctactccaacatccgtgcggccggaggggccgtctgtgagcagcccctgggcctcgagtgccgtgcccaggcccagcctggtgtccccctgcgggagttgggccaggtcgtggaatgcagcctggactttggcctggtctgcaggaaccgtgagcaggtggggaagttcaagatgtgcttcaactatgaaatccgtgtgttctgctgcaactacggccactgccccagcaccccggccaccagctctacggccatgccctcctccactccggggacgacctggatcctcacagagctgaccacaacagccactacgactgagtccactggatccacggccaccccgtcctccaccccagggaccacctggatcctcacagagccgagcactacagccaccgtgacggtgcccaccggatccacggccaccgcctcctccacccaggcaactgctggcaccccacatgtgagcaccacggccacgacacccacagtcaccagctccaaagccactcccttctccagtccagggactgcaaccgcccttccagcactgagaagcacagccaccacacccacagctaccagctttacagccatcccctcctcctccctgggcaccacctggacccgcctatcacagaccaccacacccacggccaccatgtccacagccacaccctcctccactccagagactgtccacacctccacagtgcttaccaccacggccaccacaaccggggccaccggctctgtggccaccccctcctccaccccaggaacagctcacactaccaaagtgctgactaccacaaccacgggcttcacagccaccccctcctccagcccagggacggcacgcacgcttccagtgtggatcagcacaaccaccacacccacaaccagaggttccacggtgaccccctcctccatcccggggaccacccacacccccacagtgctgaccaccaccaccacaactgtggccactggttctatggcaacaccctcctctagcacacagaccagtgggaccacccacacacccccagtgccgaacaccacggccaccacacacgggcgatccctgtcccccagcagtccccacacggtgcgcacagcctggacttcggccacctcaggcaccttgggcaccacccacatcacagagccttccacggggacttcccacaccccagcagcaaccaccggtaccacccagcactcgactccagccctgtccagccctcaccctagcagcaggaccaccgagtcacccccttctccagggacgaccaccccgggccacaccagggccacctccaggaccacggccacggccacacccagcaagacccgcacctcgaccctgctgcccagcagccccacatcggccccaataaccacggtggtgaccatgggctgtgagccccagtgtgcctggtcagagtggctggactacagctaccccatgccggggccctctggcggggactttgacacctactccaacatccgtgcggccggaggggccgtctgtgagcagcccctgggcctcgagtgccgtgcccaggcccagcctggtgtccccctgcgggagttgggccaggtcgtggaatgcagcctggactttggcctggtctgcaggaaccgtgagcaggtggggaagttcaagatgtgcttcaactatgaaatccgtgtgttctgctgcaactacggccactgccccagcaccccggccaccagctctacggccacgccctcctccactccagggacgacctggatcctcacagagcagaccacagcagccactacgaccgcaaccactggatccacggccatcccgtcctccaccccgggaacagctccccctcccaaagtgctgaccagcacggccaccacacccacagccaccagttccaaagccacttcctcctccagtccaaggactgcaaccacccttccagtgctgacaagcacagccaccaaatccacagctaccagctttacacccatcccctccttcacccttgggaccaccgggaccctcccagaacagaccaccacacccatggccaccatgtccacaatccacccctcctccactccggagaccacccacacctccacagtgctgaccacgaaggccaccacgacaagggccaccagttccatgtccaccccctcctccactccggggacgacctggatcctcacagagctgaccacagcagccactacaactgcagccactggccccacggccaccccgtcctccaccccagggaccacctggatcctcacagagcccagcactacagccaccgtgacggtgcccaccggatccacggccaccgcctcctccacccgggcaactgctggcaccctcaaagtgctgaccagcacggccaccacacccacagtcatcagctccagagccactccctcctccagtccagggactgcaaccgcccttccagcactgagaagcacagccaccacacccacagctaccagcgttacagccatcccctcttcctccctgggcaccgcctggacccgcctatcacagaccaccacacccacggccaccatgtccacagccacaccctcctctactccagagactgtccacacctccacagtgcttaccaccacgaccaccacaaccagggccaccggctctgtggccaccccctcctccaccccaggaacagctcacactaccaaagtgccgactaccacaaccacgggcttcacagccaccccctcctccagcccagggacggcactcacgcctccagtgtggatcagcacaaccaccacacccacaaccagaggctccacggtgaccccctcctccatcccggggaccacccacaccgccacagtgctgaccaccaccaccacaactgtggccactggttctatggcaacaccctcctctagcacacagaccagtgggaccacccacacacccccagtgccgaacaccacggccaccacacacgggcggtccctgccccccagcagtccccacacggtgcgcacagcctggacttcggccacctcgggcatcttgggcaccacccacatcacagagccttccacggtgacttcccacaccccagcagcaaccaccagtaccacccagcactcgactccagccctgtccagccctcaccctagcagcaggaccaccgagtcacccccttctccagggacgaccaccccgggccacaccaggggcacctccaggaccacagccacagccacacccagcaagacccgcacctcgaccctgctgcccagcagccccacatcggcccccataaccacggtggtgaccacgggctgtgagccccagtgtgcctggtcagagtggctggactacagctaccccatgccggggccctctggcggggactttgacacctactccaacatccgtgcggccggaggggcagtctgtgagcagcccctgggcctcgagtgccgtgcccaggcccagcctggtgtccccctgcgggagttgggccaggtcgtggaatgcagcctggactttggcctggtctgcaggaaccgtgagcaggtggggaagttcaagatgtgcttcaactatgaaatccgtgtgttctgctgcaactacggccactgccccagcaccccggccaccagctctacggccacgccctcctcaactccggggacgacctggatcctcacaaagctgaccacaacagccactacgactgagtccactggatccacggccaccccgtcctccaccccagggaccacctggatcctcacagagccgagcactacagccaccgtgacggtgcccaccggatccacggccaccgcctcctccacccaggcaactgctggcaccccacatgtgagcaccacggccacgacacccacagtcaccagctccaaagccactcccttctccagtccagggactgcaaccgcccttccagcactgagaagcacagccaccacacccacagctaccagctttacagccatcccctcctcctccctgggcaccacctggacccgcctatcacagaccaccacacccacggccaccatgtccacagccacaccctcctccactccagagactgcccacacctccacagtgcttaccaccacggccaccacaaccagggccaccggctctgtggccaccccctcttccaccccaggaacagctcacactaccaaagtgccgactaccacaaccacgggcttcacagtcaccccctcctccagcccagggacggcacgcacgcctccagtgtggatcagcacaaccaccacacccacaaccagtggctccacggtgaccccctcctccgtcccggggaccacccacacccccacagtgctgaccaccaccaccacaactgtggccactggttctatggcaacaccctcctctagcacacagaccagtgggaccacccacacacccccagtgccgaacaccacggccaccacacacgggcgatccctgtcccccagcagtccccacacggtgcgcacagcctggacttcggccacctcaggcaccttgggcaccacccacatcacagagccttccacggggacttcccacaccccagcagcaaccaccggtaccacccagcactcgactccagccctgtccagccctcaccctagcagcaggaccaccgagtcacccccttccccagggacgaccaccccgggccacaccacggccacctccaggaccacggccacggccacacccagcaagacccgcacctcgaccctgctgcccagcagccccacatcggcccccataaccacggtggtgaccacgggctgtgagccccagtgtgcctggtcagagtggctggactacagctaccccatgccggggccctctggcggggactttgacacctactccaacatccgtgcggccggaggggccgtctgtgagcagcccctgggcctcgagtgccgtgcccaggcccagcctggtgtccccctgggggagttgggccaggtcgtggaatgcagcctggactttggcctggtctgcaggaaccgtgagcaggtggggaagttcaagatgtgcttcaactatgaaatccgtgtgttctgctgcaactacggccactgccccagcaccccggccaccagctctacggccatgccctcctccactccggggacgacctggatcctcacagagctgaccacaacagccactacgactgcatccactggatccacggccaccccgtcctccaccccgggaacagctccccctcccaaagtgctgaccagcccggccaccacacccacagccaccagttccaaagccacttcctcctccagtccaaggactgcaaccacccttccagtgctgacaagcacagccaccaaatccacagctaccagcgttacacccatcccctcctccacccttgggaccaccgggaccctcccagaacagaccaccacacccgtggccaccatgtccacaatccacccctcctccactccggagaccacccacacctccacagtgctgaccacgaaggccaccacgacaagggccaccagttccacgtccaccccctcctccactccggggacgacctggatcctcacagagctgaccacagcagccactacaactgcagccactggccccacggccaccccgtcctccaccccagggaccacctggatcctcacagagctgaccacaacagccactacgactgcgtccactggatccacggccaccccgtcctccaccccagggaccacctggatcctcacagagccgagcactacagccaccgtgacggtgcccaccggatccacggccaccgcctcctccacccaggcaactgctggcaccccacatgtgagcaccacggccacgacacccacagtcaccagctccaaagccactccctcctccagtccagggactgcaactgcccttccagcactgagaagcacagccaccacacccacagctaccagctttacagccatcccctcctcctccctgggcaccacctggacccgcctatcacagaccaccacacccacggccaccatgtccacagccacaccctcctccactccagagactgtccacacctccacagtgcttaccgccacggccaccacaaccggggccaccggctctgtggccaccccctcctccaccccaggaacagctcacactaccaaagtgccgactaccacaaccacgggcttcacagccaccccctcctccagcccagggacggcactcacgcctccaaccaccacacccatgtccaccatgtccacaatccacacctcctctactccagagaccacccacacctccacagtgctgaccaccacagccaccatgacaagggccaccaattccacggccacaccctcctccactctggggacgacccggatcctcactgagctgaccacaacagccactacaactgcagccactggatccacggccaccctgtcctccaccccagggaccacctggatcctcacagagccgagcactatagccaccgtgatggtgcccaccggttccacggccaccgcctcctccactctgggaacagctcacacccccaaagtggtgaccaccatggccactatgcccacagccactgcctccacggttcccagctcgtccaccgtggggaccacccgcacccctgcagtgctccccagcagcctgccaaccttcagcgtgtccactgtgtcctcctcagtcctcaccaccctgagacccactggcttccccagctcccacttctctactccctgcttctgcagggcatttggacagtttttctcgcccggggaagtcatctacaataagaccgaccgagccggctgccatttctacgcagtgtgcaatcagcactgtgacattgaccgcttccagggcgcctgtcccacctccccaccgccagtgtcctccgccccgctgtcctcgccctcccctgcccctggctgtgacaatgccatccctctccggcaggtgaatgagacctggaccctggagaactgcacggtggccaggtgcgtgggtgacaaccgtgtcgtcctgctggacccaaagcctgtggccaacgtcacctgcgtgaacaagcacctgcccatcaaagtgtcggacccgagccagccctgtgacttccactatgagtgcgagtgtgagtgcgtcggtggccgcgggattaccccgggggcaggcatctgcagcatgtggggcggctcccactattccacctttgacggcacctcttacaccttccggggcaactgcacctatgtcctcatgagagagatccatgcacgctttgggaatctcagcctctacctggacaaccactactgcacggcctctgccactgccgctgccgctgccgcccgctgcccccgcgccctcagcatccactacaagtccatggatatcgtcctcactgtcaccatggtgcatgggaaggaggagggcctgatcctgtttgaccaaattccggtgagcagcggtttcagcaagaacggcgtgcttgtgtctgtgctggggaccaccaccatgcgtgtggacattcctgccctgggcgtgagcgtcaccttcaatggccaagtcttccaggcccggctgccctacagcctcttccacaacaacaccgagggccagtgcggcacctgcaccaacaaccagagggacgactgtctccagcgggacggaaccactgccgccagttgcaaggacatggccaagacgtggctggtccccgacagcagaaaggatggctgctgggccccgactggcacaccccccactgccagccccgcagccccggtgtctagcacacccacccccaccccatgcccaccacagccgctctgtgatctgatgctgagccaggtctttgctgagtgccacaaccttgtgcccccgggcccattcttcaacgcctgcatcagcgaccactgcaggggccgccttgaggtgccctgccagagcctggaggcttacgcagagctctgccgcgcccggggagtgtgcagtgactggcgaggtgcaaccggtggcctgtgcgacctcacctgcccacccaccaaagtgtacaagccatgcggccccatacagcctgccacctgcaactctaggaaccagagcccacagctggaggggatggcggagggctgcttctgccctgaggaccagatcctcttcaacgcacacatgggcatctgcgtgcaggcctgcccctgcgtgggacccgatgggtttcctaaatttcccggggagcggtgggtcagcaactgccagtcctgcgtgtgtgacgagggttcagtgtcggtgcagtgcaagcccctgccctgtgacgcccagggtcagcccccgccgtgcaaccgtcccggcttcgtaaccgtgaccaggccccgggccgagaacccctgctgccccgagacggtgtgcgtgtgcaacacaaccacctgcccccagagcctgcctgtgtgcccgccagggcaggagtccatctgcacccaggaggagggcgactgctgtcccaccttccgctgcagacctcagctgtgttcgtacaatggcaccttctacggggttggtgcaaccttcccaggcgcccttccctgccacatgtgtacctgcctctctggggacacccaggacccaacggtgcaatgtcaggaggatgcctgcaacaatactacctgtccccagggctttgagtacaagagagtggccgggcagtgctgtggggagtgcgtccagaccgcctgcctcacgcccgatggccagccagtccagctgaatgaaacctgggtcaacagccatgtggacaactgcaccgtgtacctctgtgaggctgagggtggagtccatttgctgaccccacagcctgcatcctgcccagatgtgtccagctgcagggggagcctcaggaaaaccggctgctgctactcctgtgaggaggactcctgtcaagtccgcatcaacacgaccatcctgtggcaccagggctgcgagaccgaggtcaacatcaccttctgcgagggctcctgccccggagcgtccaagtactcagcagaggcccaggccatgcagcaccagtgcacctgctgccaggagaggcgggtccacgaggagacggtgcccttgcactgtcctaacggctcagccatcctgcacacctacacccacgctgtccaggtcctctgtgggctgcttgcctggggcctgcaggctggaggacacatccggggggcagtccaggacccccagcagccactgaaggaccaggaagcctctgggaaggccaggcaggggggtggctacaggcagaccgtggcctggggagacaaaagcaatgctcgtgcctggctgcagaagcctgtggtgtgggtgcagagcggggccttccccacaccggggcctgcctctgccctgtgcccttggaaaatgggcattcagcctgaaaccaccaagcagctcagagatgctaacatcctgaaggagagcaaacgctccatcagtagagaaagacaaaggcaatgtgctcaagctatcaggtttaatagaggatttggaggacagatctggaaatctcaacgctttttctag

SEQ ID NO: 2 - DNA, 19 bp, Artificial Sequence
  Synthetic Construct
cctgcaagcctccaggtag

SEQ ID NO: 3 - DNA, 18 bp, Artificial Sequence
  Synthetic Construct
ctgctccactggctttgg

SEQ ID NO: 4 - DNA, 24 bp, Artificial Sequence
  Synthetic Construct
ctgagctgcttggtggtttcaggc

SEQ ID NO: 5 - DNA, 24 bp, Artificial Sequence
  Synthetic Construct
gagctgcttggtggtttcaggctg

SEQ ID NO: 6 - DNA, 25 bp, Artificial Sequence
  Synthetic Construct
ctgcttggtggtttcaggctgaatg

SEQ ID NO: 7 - DNA, 25 bp, Artificial Sequence
  Synthetic Construct
ttccaagggcacagggcagaggcag

SEQ ID NO: 8 - DNA, 25 bp, Artificial Sequence
  Synthetic Construct
gctgcttggtggtttcaggctgaat

SEQ ID NO: 9 - DNA, 24 bp, Artificial Sequence
  Synthetic Construct
gccactgaaggaccaggaagcctc

SEQ ID NO: 10 - DNA, 22 bp, Artificial Sequence
  Synthetic Construct
ccagcagccactgaaggaccag

SEQ ID NO: 11 - DNA, 20 bp, Artificial Sequence
  Synthetic Construct
ggctggaggacacatccggg

SEQ ID NO: 12 - DNA, 25 bp, Artificial Sequence
  Synthetic Construct
ctgcaggctggaggacacatccggg

SEQ ID NO: 13 - DNA, 25 bp, Artificial Sequence
  Synthetic Construct
cctgcaggctggaggacacatccgg

SEQ ID NO: 14 - DNA, 25 bp, Artificial Sequence
  Synthetic Construct
ggcctgcaggctggaggacacatcc

SEQ ID NO: 15 - DNA, 25 bp, Artificial Sequence
  Synthetic Construct
gggcctgcaggctggaggacacatc

SEQ ID NO: 16 - DNA, 25 bp, Artificial Sequence
  Synthetic Construct
agaagcctgtggtgtgggtgcagag

SEQ ID NO: 17 - DNA, 18 bp, Artificial Sequence
  Synthetic Construct
ctctgcacccacaccaca

SEQ ID NO: 18 - DNA, 18 bp, Artificial Sequence
  Synthetic Construct
aggaagcctctgggaagg

SEQ ID NO: 19 - DNA, 20 bp, Artificial Sequence
  Synthetic Construct
tggtcctcagagatgggaag

SEQ ID NO: 20 - DNA, 20 bp, Artificial Sequence
  Synthetic Construct
gaaaccccaatagcccattc